(19) United States
(12) Patent Application Publication
(10) Pub. No.: US 2009/0233986 A1
(43) Pub. Date: Sep. 17, 2009

(54) Methods and compositions for using sax2

(75) Inventors: Ruth Simon, New York, NY (US); Thomas Lufkin, Singapore (SG)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: MOUNT SINAI SCHOOL OF MEDICINE, New York, NY (US)

(21) Appl. No.: 11/658,460

(22) PCT Filed: Jul. 22, 2005
(86) PCT No.: PCT/US05/26034

§ 371 Date: Feb. 4, 2009

Related U.S. Application Data
(60) Provisional application No. 60/591,450, filed on Jul. 27, 2004.

(30) Foreign Application Priority Data
Jul. 27, 2004 (US) 60/591450

(51) Int. Cl. A61K 31/7052; C07H 21/00; C12N 15/63; C12N 5/00; C12N 1/21; C12N 1/19; C12N 5/06; C07K 14/00; C07K 7/06; C07K 7/08; C07K 16/00; A61P 3/00

(52) U.S. Cl. 514/44 R; 536/23.5; 435/320.1; 435/325; 435/252.3; 435/254.2; 435/348; 530/350; 530/328; 530/327; 530/326; 530/325; 530/324; 530/387.9; 435/375

(57) ABSTRACT

The present invention is generally directed to factors involved in energy metabolism. More specifically, a new gene is identified and shown to be a homeobox Sax2 gene that is involved with an obesity phenotype. Deletion of the causes growth retardation starting at birth and high postnatal lethality. Further, abrogation of the gene leads to a lack of fat accumulation in white adipose tissue and brown adipose tissue, as well as low blood glucose levels. Methods and compositions for making and using the product of the gene as well as for the manufacture of medicament for the treatment of obesity and obesity-related disorders are described.

### BACKGROUND

1. Field of the Invention

The present invention is generally directed to factors involved in energy metabolism. More specifically, a new gene is identified and shown to be a homeobox gene that is involved with an obesity phenotype. Abrogation of the gene leads to lack of fat accumulation in adipose tissue and low blood glucose levels.

2. Background of the Related Art

The Center for Disease Control (CDC) defines obesity as having a very high amount of body fat in relation to lean body mass, or Body Mass Index (BMI) value of 30 or higher. The BMI of an individual is the measure of an adult's weight in relation to his/her height, i.e., the weight of an adult in kilograms divided by the square of adult's height in meters. The CDC's Behavioral Risk Factor Surveillance System annually provides data relating to obesity in the United States. These statistics show that in 1991 there was an average of 10-14% obesity in the majority of the United States, this figure had increased markedly to between 20-24% obesity in the majority of the United States by the year 2002 and continues to increase.

Adipocyte tissue plays an important role in energy homeostasis. White adipocyte tissue (WAT) is not only the major site for energy storage in the form of triglycerides and lipids, but also plays an important role in the regulation of energy balance by secreting hormones, e.g. leptin. Brown adipocyte tissue (BAT) has a major function in the regulation of thermogenesis by virtue of the mitochondrial protein uncoupling protein-1 (UCP-1) (reviewed in Lowell and Spiegelman, Nature 404: 652-660, 2000). Thus, the two types of adipose tissue, BAT and WAT, carry out very different roles in the body. White adipose is designed to store excess caloric intake while brown adipose tissue uses a unique system to use excess calories to generate body heat. The heat is generated in the mitochondria of brown adipose where oxidation of substrate is utilized to create a hydrogen ion gradient that is then collapsed in a regulated fashion generating heat instead of ATP. It has been shown that transgenic animals that lack brown adipose maintain efficient metabolism, are obese and continue to overeat. Other rodent studies have also shown a link between obesity, continued overeating and sensitivity to cold, suggesting a connection to the sympathetic nervous system.

Both BAT and WAT originate from mesodermal stem cells during embryonic development. Adipogenesis starts late in embryogenesis as a preparation for postnatal life. In mice, WAT is almost completely absent at birth and develops postnatally (Slavin, Anat. Rec. 195: 63-72, 1979) while BAT develops earlier during late embryogenesis.

There are three major transcription factor families that are involved in the differentiation of adipocytes, peroxisome proliferator-activator receptor (PPAR), CCAAT/enhancer-binding protein (C/EBP) and adipocyte determination and differentiation dependant factor 1 (ADD-1) or sterol regulatory element-binding protein 1 (SREBP-1) (reviewed in Rangwala and Lazar, Annu. Rev. Nutr. 20: 535-559, 2000). Deletion of these factors leads to loss of adipocyte differentiation. As mentioned above WAT plays a major function in the regulation of energy balance by its secreting factors, termed adipocytokines, which include leptin (Friedman, Nature 404: 632-634, 2000), adiponectin (Yamauchi et al, Nature Med. 7: 941-946, 2001) and tumor necrosis factor (TNF)-α (Hotamisligil, J. Intern Med. 245: 621-625, 1999). Leptin arose as one of the major factors involved in energy homeostasis secreted by the adipocyte tissue. Together with insulin, leptin plays a well-defined critical role in adipogenesis and energy homeostasis. Leptin and insulin are not only active in adipocyte tissue but also in the brain, particularly in the hypothalamus, a crucial region for regulation of energy homeostasis (reviewed in Schwartz et al, Nature 404: 661-671, 2000; Flier, Cell 116: 337-350, 2004).

While leptin is expressed predominantly in WAT, insulin is secreted by the islets of Langerhans in the pancreas and both play regulatory roles in specific nuclei of the hypothalamus. In particular, leptin and insulin regulate NPY and POMC neurons in the arcuate nucleus through distinct pathways. In brief, high levels of leptin and insulin prevent food intake by suppressing NPY and agouti-related protein (AgRP) expression in the NPY neuron and activating POMC and cocaine- and amphetamine-regulated transcript (CART) expression in the POMC neurons. Low leptin and insulin levels, on the other hand, activate NPY and AgRP expression and inhibit POMC and CART expression that increases appetite and can cause obesity.

In addition to the hypothalamus, energy balance is also regulated by serotonergic neurons. Although a specific neural circuitry has not yet been established, leptin receptors are located on serotonergic neurons in the dorsal raphe nucleus suggesting a role for leptin in the regulation of food uptake through these neurons (Collin et al, Mol. Brain. Res. 81: 51-61, 2000). It has also been reported that mutations in the serotonergic 5-HT2C-receptor gene causes hyperphagia and diabetes mellitus independently from leptin, further establishing a role for serotonergic neurons in energy homeostasis (Nonogaki et al, Nature Med. 4: 1152-1156, 1998).

As discussed in Villafuerte et al. (Obesity Research, 8 (9): 646, 2000), WAT is one of the most metabolically active organs in the body and many investigators have reported region-specific differences in the cellularity, physiology, and metabolic behavior of anatomically discrete fat depots during expansion of the adipose mass and the development of obesity (Ailhaud et al., Int J Obes 15, 87-90, 1991; Bjömtorp, Int J Obes Relat Metab Disord 20,291-302, 1996; Lefebvre et al., Diabetes 47, 98-103, 1998; Newby et al., Am J Physiol 255, E716-E722, 1988; Warden et al., J Clin Invest 95, 1545-1552, 1995). While the role of blood circulating factors involved in the regulation of the glucose and fat metabolism, e.g. insulin and leptin, and local factors, such as, angiotensinogen, interleukin-6, tumor necrosis factor-, insulin-like growth factor-1 (IGF-1), and leptin (Lefebvre et al., Diabetes 47, 98-103, 1998; Arner, Lancet 351, 1301-1302, 1998; Frederich, et al., Hypertension 19, 339-344, 1992; Hotamisligil, et al., J Clin Invest 95, 2409-2415, 1995; Mohamed-Ali, et al., Int J Obes 22, 1145-1158, 1998; Montague et al., Diabetes 46, 342-347, 1997; Safonova et al., Biochem J 322, 235-239, 1997) present in WAT has been acknowledged, there is a recognition in the art for the need to identify other factors that play a role in WAT regulation in obesity and obesity-related disorders. See Villafuerte et al. (Obesity Research, 8 (9): 646, 2000).

Thus, imbalance in energy metabolism in the body leads to several diseased states, most notably obesity and obesity-induced diabetes and these can be described as dysfunctions of energy storage tissues. The role of WAT and BAT in energy metabolism is crucial and it is recognized that there is a need to identify further factors that regulate the role of these tissues in order to provide a better entry point for the amelioration, prevention or intervention in disorders of energy metabolism.

### SUMMARY OF THE INVENTION

The present application for the first time provides a teaching of an isolated recombinant nucleic acid encoding a SAX2 polypeptide wherein the polypeptide is expressed in brain tissue, the polypeptide being encoded by the nucleic acid sequence presented in SEQ ID NO: 1, SEQ ID NO:3, SEQ ID NO:4, or SEQ ID NO:8. In specific aspects, there is a teaching of an isolated recombinant nucleic acid encoding a recombinant protein having the amino acid sequence of SEQ ID NO:2 or SEQ ID NO:10. Also contemplated is an isolated recombinant nucleic acid comprising the sequence presented in SEQ ID NO: 1 (for mouse) or SEQ ID NO:3, (for human) the mature protein coding portion thereof, or a complement thereof. Also provided is an isolated recombinant nucleic acid encoding a polypeptide of SEQ ID NO: 2 or SEQ ID NO:10. In certain embodiments, the isolated nucleic acid is a genomic genomic DNA, in other embodiments, the nucleic acid is cDNA. Also taught herein is an isolated polynucleotide that encodes a SAX2 protein and hybridizes under high stringency conditions to a nucleic acid of any of claims 1, 2, 3, or 4 but does not hybridize to a sequence that encodes SAX-1.

In specific aspects of the invention, there is contemplated a compound 8 to 80 nucleotides in length targeted to a nucleic acid molecule encoding SAX2, wherein the compound specifically hybridizes with a nucleic acid molecule of SEQ ID NO:1 (murine) or SEQ ID NO:3 (human) and inhibits the expression of SAX2. Such a compound may be 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more nucleotides in length and may be generated to be complementary to and hybridize with any portion of SEQ ID NO:1 or SEQ ID NO:3. With the knowledge of the sequence of SEQ ID NO:1 and SEQ ID NO:3, such compounds may now be readily generated using standard techniques. Preferably, the compound is 12 to 50 nucleotides in length. Even more preferably, the compound is 15 to 30 nucleotides in length. In specific embodiments, the compound of is 20 to 25 nucleotides in length. The use of such compounds for gene silencing is particularly contemplated. In preferred embodiments, the compound is an antisense oligonucleotide. In specific embodiments, the compound is a DNA oligonucleotide. In other embodiments, the compound is an RNA oligonucleotide. In specific embodiments, at least a portion of the compound hybridizes with RNA to form an oligonucleotide-RNA duplex. In other particular embodiments, the compound is one which has at least 70% complementarity with a nucleic acid molecule of SEQ ID NO 1 wherein the compound specifically hybridizes to and inhibits the expression of SAX2. Preferably, the compound has at least 80% complementarity with a nucleic acid molecule of SEQ ID NO 1 wherein the compound specifically hybridizes to and inhibits the expression of SAX2. In other embodiments, the compound has at least 90% complementarity with a nucleic acid molecule of SEQ ID NO 1 wherein the compound specifically hybridizes to and inhibits the expression of SAX2. In still further embodiments, the compound is one which has at least 95% complementarity with a nucleic acid molecule of SEQ ID NO 1 wherein the compound specifically hybridizes to and inhibits the expression of SAX2. In certain aspects of the invention, the compound has at least one modified internucleoside linkage, sugar moiety, or nucleotide.

The invention further contemplates an expression construct comprising an isolated nucleic acid encoding a protein having an amino acid sequence of SEQ ID NO:2 or SEQ ID NO:10 or the mature protein portion thereof and a promoter operably linked to the polynucleotide. In preferred embodiments, the expression construct is one in which the nucleic acid comprises a mature protein coding sequence as set forth in SEQ ID NO:1 (mouse) or SEQ ID NO:3 (human). In preferred embodiments, the expression construct is an expression construct selected from the group consisting of an adenoassociated viral construct, an adenoviral construct, a herpes viral expression construct, a vaccinia viral expression construct, a retroviral expression construct, a lentiviral expression construct and a naked DNA expression construct.

The invention further is directed to a recombinant host cell stably transformed or transfected with a nucleic acid of the present invention in a manner allowing the expression in the host cell of a protein of SEQ ID NO:2 or SEQ ID NO:10. In specific embodiments, the recombinant host cell is transfected with a nucleic acid having the sequence as set forth in SEQ ID NO:1 (mouse) or SEQ ID NO:3 (human). Also contemplated are recombinant host cells stably transformed or transfected with an expression construct as described herein, in a manner allowing the expression in the host cell of a protein of SEQ ID NO:2 or SEQ ID NO:10. In certain embodiments, the host cell is a mammalian cell, a bacterial cell, a yeast cell, or an insect cell.

Also taught herein is an isolated and purified protein comprising an amino acid sequence as set forth in SEQ ID NO:2, or SEQ ID NO:10, or the mature protein portion thereof. In addition, specifically contemplated herein are proteins that have an amino acid sequence that is 90% identical to the sequence set forth in SEQ ID NO:2 or SEQ ID NO:10. In addition, the invention further contemplated fragments of SEQ ID NO:2 that retain one or more biological properties of a protein of SEQ ID NO:2 or SEQ ID NO:10. Other embodiments contemplate an isolated and purified peptide comprising about 10 to about 50 contiguous amino acids of SEQ ID NO:2 or SEQ ID NO:10.

The present invention is further directed to diagnostic kit for detecting a nucleic acid that encodes a SAX2 polypeptide, the polypeptide being encoded by the sequence presented in SEQ ID NO: 1, comprising an isolated nucleic acid probe complementary to the complete sequence of SEQ ID NO: 1, and a container for containing the nucleic acid. Further, the invention is directed to a purified antibody that is specifically immunoreactive with the protein of the invention. The antibody may be a monoclonal antibody. Also contemplated is a monoclonal antibody that is specifically immunoreactive with a protein of the present invention.

Further embodiments are directed to methods of identifying a modulator of SAX2 expression identified by a method comprising the steps of contacting a cell that expresses SAX2 with the candidate modulator substance; monitoring the expression of SAX2; and comparing the expression of SAX2 in the presence and absence of the candidate substance; wherein an alteration in the expression of SAX2 indicates that the substance is a modulator of SAX2 expression. In specific embodiments, the modulator of SAX2 expression comprises an oligonucleotide, an antisense oligonucleotide, a DNA oligonucleotide, an RNA oligonucleotide, an RNA oligonucleotide having at least a portion of the RNA oligonucleotide capable of hybridizing with RNA to form an oligonucleotide-RNA duplex, or a chimeric oligonucleotide.

The present invention further provides methods of producing a purified SAX2 protein comprising preparing an expression construct comprising a nucleic acid of SEQ ID NO:1 (murine) or SEQ ID NO:3 (human) operably linked to a promoter; transforming or transfecting a host cell with the expression construct in a manner effective to allow the expression of a protein having an amino acid sequence of SEQ ID NO:2 or SEQ ID NO:10, or the mature protein portion thereof in the host cell; culturing the transformed or transfected cell under conditions to allow the production of the protein by the transformed or transfected host cell; and isolating the SAX2 protein from the host cell.

Other embodiments are directed to a composition that comprises expression construct comprising an isolated polynucleotide encoding a SAX2 protein having a sequence of SEQ ID NO:2 or SEQ ID NO:10 or a biologically active fragment thereof and a promoter operably linked to the polynucleotide; and a pharmaceutically acceptable carrier excipient or diluent. Other embodiments contemplate compositions comprising an isolated and purified SAX2 polypeptide of SEQ ID NO:2 or SEQ ID NO:10, or a biologically active fragment of SEQ ID NO:2 or SEQ ID NO:10 and a pharmaceutically acceptable carrier, diluent or excipient.

The invention is further directed to methods of inhibiting the expression of SAX2 in cells or tissues comprising contacting the cells or tissues with a compound that is about 8 to 50 nucleotides in length targeted to a nucleic acid molecule encoding SAX2, wherein said compound specifically hybridizes with a nucleic acid molecule of SEQ ID NO:1 (or SEQ ED NO:3) and inhibits the expression of SAX2 such that that expression of SAX2 is inhibited as a result of the contacting with said compound.

Also contemplated herein is a method of decreasing fat deposition in a mammal comprising inhibiting the expression or activity of SAX2 in the mammal. In preferred embodiments, the decrease in fat deposition manifests as a decrease in the white adipocyte tissue (WAT) of the mammal, a decrease in the brown adipocyte tissue (BAT) of the mammal, or a decrease in both WAT and BAT upon inhibition of expression or activity of SAX2 in the mammal.

Other embodiments provide methods of treating or ameliorating diabetes mellitus which comprises administering to a mammal afflicted with diabetes a composition that inhibits the expression and/or activity of a mammalian SAX2 polypeptide, the polypeptide having the sequence of SEQ ID NO:2 or SEQ ED NO:10.

In yet another embodiment, there is a method of inhibiting adipogenesis in comprising contacting a population of preadipocytes with an inhibitor of SAX2 activity, wherein inhibition of SAX2 activity inhibits the differentiation of the preadipbcytes into WAT. In certain embodiments, the population of preadipocytes are in vitro. In other embodiments, the preadipocytes are in vivo.

Also provided is a method for treating an obesity related disorder in an obese mammal comprising suppressing precursor adipocyte differentiation in the obese mammals by administering to the obese mammal an effective amount of a composition that inhibits the expression or activity of SAX2 in an amount effective to inhibit the differentiation of adipocytes. In certain aspects, the method comprises suppressing the production of WAT in the animal. In other aspects, the method comprises suppressing the production of BAT in the animal. The methods for the treatment of obesity contemplated herein may comprising administering to the mammal a therapeutically or prophylactically effective amount of the compound a compound that is about 8 to 50 nucleotides in length targeted to a nucleic acid molecule encoding SAX2, wherein said compound specifically hybridizes with a nucleic acid molecule of SEQ ID NO:1 (or SEQ ID NO:3) and inhibits the expression of SAX2 such that expression of SAX2 is inhibited. Preferably, the mammal is a human.

The invention is further directed to a transgenic mouse comprising a disrupted SAX2 gene, wherein the transgenic mouse is homozygous for the disrupted SAX2 gene, and wherein the transgenic mouse exhibits a phenotype in which there is a decrease in WAT as compared to non-transgenic animals of the same lineage.

Also provided herein are methods of making a transgenic mouse having a disrupted SAX2 gene, comprising providing a murine embryonic stem cell comprising an intact SAX2 gene having a sequence of SEQ ID NO:1; providing a targeting vector capable of disrupting the SAX2 gene upon homologous recombination; introducing the targeting vector into the murine embryonic stem cell under conditions where the targeting vector will undergo homologous recombination with the SAX2 gene of the murine embryonic stem cell to produce a disrupted gene; introducing the murine embryonic stem cell into a blastocyst; implanting the blastocyst into a pseudopregnant female mouse; and delivering a first transgenic mouse comprising a disrupted SAX2 gene from the pseudopregnant female; repeating the above steps to obtain a second transgenic mouse comprising a disrupted SAX2 gene; and breeding the first transgenic mouse comprising a disrupted SAX2 gene to the second transgenic mouse comprising a disrupted SAX2 gene to obtain one or more mice homozygous for a disrupted SAX2 gene.

The invention is further directed to a murine cell line comprising a disrupted SAX2 gene, wherein substantially all cells of the cell line have both copies of the SAX2 gene disrupted.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1A and FIG. 1B show hematoxylin and Eosin stained paraffin section of adipocyte tissue. (FIG. 1A) HE staining of epididymal and mesenteric WAT. (FIG. 1B) HE staining of WAT and BAT complex of the neck region (right panel shows cell at higher magnification).

FIG. 2A and FIG. 2B shows an alignment of the human SAX2 genomic DNA, cDNA and protein in the human seq map.doc.

FIG. 3 shows an alignment of the murine SAX2 genomic DNA, cDNA and protein.

### DESCRIPTION OF THE PREFERRED EMBODIMENTS

In 1999-2000, an estimated 30% of U.S adults aged 20 years and older, nearly 59 million people, were obese, defined as having a body mass index (BMI) of 30 or more (National Health and Nutrition Examination Survey 1999-2000). The figure is equally alarming for children. In the same survey, it was estimated that 15% of children and adolescents aged 6-19 years were overweight, defined as a body mass index for age and sex at or above the 95th percentile of the CDC growth charts. Obesity-related disorders, such as obesity-related diabetes, heart disease, stroke, cancer (such as colon cancer, endometrial cancer, and postmenopausal breast cancer), gallbladder disease, sleep apnea (interrupted breathing during sleep), osteoarthritis (wearing away of the joints) also are on the increase. The obesity epidemic presents an urgent need to identify new methods for the prevention, treatment, or amelioration of obesity and its related disorders.

Herein is described a previously uncharacterized homeobox gene, Sax2 that shows high similarity to the Drosophila S59/slouch and the murine Sax1 genes. Sax2 gene expression occurs early during embryogenesis in the midbrain, the mid/hindbrain boundary, the ventral neural tube, the developing eye and the apical ectodermal ridge of the limb. The role of Sax2 during development was investigated by generating a knockout mouse line by replacing part of the Sax2 coding sequences with the lacZ gene. The Sax2 mutants exhibit a strong phenotype indicated by growth retardation starting immediately after birth and leading to premature death within the first 3 weeks postnatal. Intriguingly, the studies also demonstrated a striking autoregulation of the Sax2 gene in both a positive and a negative feedback mechanism depending on the specific cell type expressing Sax2.

In addition to the above studies, further analysis of the Sax2 null mutants revealed lack of fat accumulation in WAT and BAT and low blood glucose levels. Given the defect in adipogenesis, and the expression of Sax2 in the brain, the present invention is directed to the critical function of Sax2 in the regulation of energy balance and energy homeostasis. As it is demonstrated herein that lack or decreased expression of the Sax2 gene leads to a decrease in fat deposition/accumulation in adipocytes, the present invention in certain aspects provides methods and compositions for the treatment of obesity which involve decreasing the expression and/or activity of Sax2 gene/gene product. Conversely, the Sax2 gene expression may be increased in those animals/subjects in which it would be desirable to produce an increase in fat deposition/accumulation (e.g., in anorexics, or in farm animals where it is desirable to produce fatter animals). Methods and compositions for using Sax2 and Sax2-related compositions in these and other indications are described in further detail below.

### Polypeptide and Fragments Thereof.

According to the present invention, there has been identified a Sax2 gene. It is identified herein as a homeobox gene that is expressed predominantly in the mid/hindbrain boundary and the neural tube early during development. Deletion of Sax2 causes growth retardation starting at birth and high postnatal lethality within the first 3 weeks after birth, and there is also a lack of fat accumulation in adipocytes when the gene is deleted. The functional aspects of the gene expression are discussed in further detail below. However, given the role of the gene in adipogenesis, it is contemplated that it will be desirable to inhibit, decrease, ablate, reduce or otherwise diminish the expression of the gene or the activity of the protein product of the gene expression. It is contemplated that inhibition of the expression of this gene will have a beneficial effect in treating obesity and obesity-related disorders. In such aspects, guidance may be gained from the functional and therapeutic aspects of leptin, another gene recognized as being involved in obesity (see e.g., U.S. Pat. No. 6,734,160; U.S. Pat. No. 6,703,493; U.S. Pat. No. 6,471,956; U.S. Pat. No. 6,429,290; U.S. Pat. No. 6,350,730; U.S. Pat. No. 6,309,853; U.S. Pat. No. 6,124,448; U.S. Pat. No. 6,124,439; U.S. Pat. No. 6,048,837; U.S. Pat. No. 6,001,968; U.S. Pat. No. 5,976,082; U.S. Pat. No. 5,935,810; U.S. Pat. No. 5,939,387; U.S. Pat. No. 5,851,995; U.S. Pat. No. 5,719,266; U.S. Pat. No. 5,691,309; U.S. Pat. No. 5,605,886; U.S. Pat. No. 5,594,104; U.S. Pat. No. 5,580,954; U.S. Pat. No. 5,574,133; U.S. Pat. No. 5,569,744; U.S. Pat. No. 5,569,743; U.S. Pat. No. 5,567,803; U.S. Pat. No. 5,567,678; U.S. Pat. No. 5,563,245; U.S. Pat. No. 5,563,244; U.S. Pat. No. 5,563,243; U.S. Pat. No. 5,559,208; U.S. Pat. No. 5,554,727; U.S. Pat. No. 5,552,524; U.S. Pat. No. 5,552,523; U.S. Pat. No. 5,552,522; U.S. Pat. No. 5,532,336; U.S. Pat. No. 5,525,705; U.S. Pat. No. 5,521,283, each incorporated by reference). In the present application, the inventors showed that this protein is expressed in mid/hindbrain boundary and the ventral midbrain as well as the ventral neural tube, suggesting that the Sax2 gene product is an endocrine factor. While treatment of obesity and obesity-related disorders (e.g., hypertension and other heart disease, diabetes, strokes, and the like) will generally involve inhibition of the Sax2 gene, it is contemplated that in certain embodiments, it will be desirable to increase the expression of Sax2 in disorders which may benefit from an increase in fat deposition (e.g., disorders in which the individual has a pronounced weight loss, e.g., anorexia nervosa, bulimia and the like).

An additional embodiment in which it would be desirable to increase, augment or otherwise supplement endogenous Sax2 expression and/or activity is in commercial or experimental endeavors where it would be desirable to produce animals/subjects that have an increased Sax2 expression and are phenotypically obese. For example, it may be desirable to produce farm or other animals that are larger than the average animal. Alternatively, it may be desirable to produce a genetically obese mouse to serve as models for obesity and insulin resistance. Leptin deficient mice, such as e.g., the ob/ob mutant mice have previously been created as such models. Methods of increasing, augmenting or supplementing endogenous activity may involve supplying to a cell or an organism a composition comprising an isolated polypeptide encoding a Sax2 protein. Such protein-based compositions are discussed in further detail herein below.

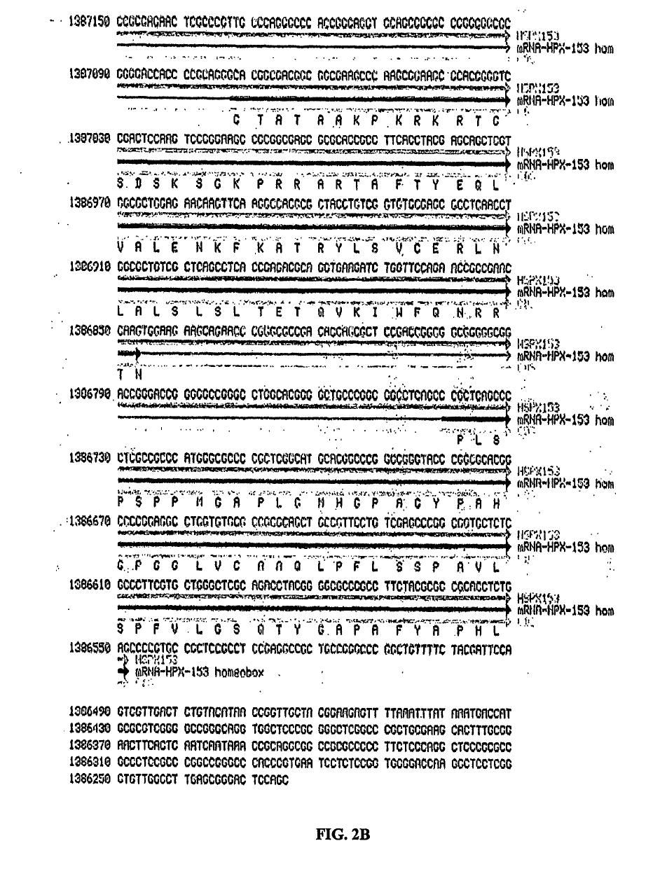

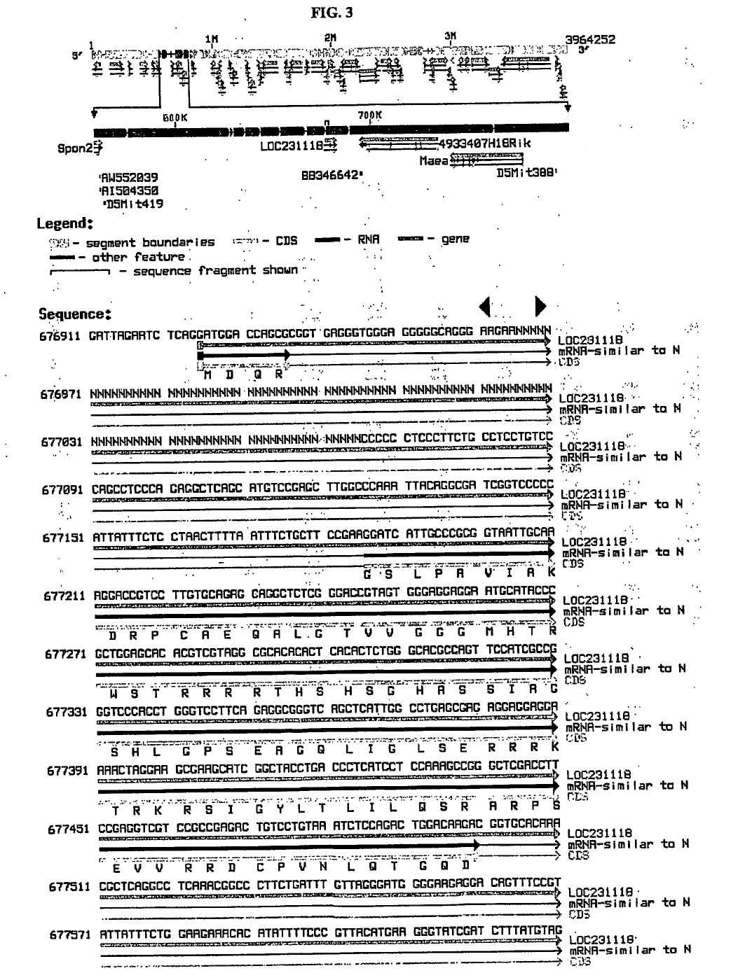

The murine Sax2 gene has been cloned by the present inventors and is taught herein to have a nucleic acid sequence as shown in SEQ ID NO:1. The coding region of the murine Sax2 gene encodes a protein of SEQ ID NO:2. The human sequence is provided in SEQ ID NO:3. The predicted protein encoded by the human sequence is shown is SEQ ID NO:10. FIG. 2 shows an alignment ment of the human SAX2 genomic DNA, cDNA and protein in the human seq map.doc. SEQ ID NO:4 sets forth a partial cDNA sequence that encodes a partial murine Sax2 protein of SEQ ID NO:5. SEQ ID NO:8 provides a further partial sequence of the murine Sax2 cDNA, which encodes a protein of SEQ ID NO:9. The transcription factor activity of any of these factors may be readily tested using techniques well known to those of skill in the art. Further, the functional activity of these agents as modulators of body weight also may be readily assessed.

In addition to the entire Sax2 protein molecule of SEQ ID NO:2, the compositions of the present invention also may employ fragments of the polypeptide that may or may not retain the biological activity of Sax2 protein. Fragments, including the N-terminus or C terminus of the molecule may be generated by genetic engineering of translation start or stop sites within the coding region (discussed below). Alternatively, treatment of the Sax2 protein molecule with proteolytic enzymes, known as proteases, can produce a variety of N-terminal, C-terminal and internal fragments. Examples of fragments may include contiguous residues of the Sax2 protein sequence of SEQ ID NO:2 or SEQ ID NO:10, of 6, 7, 8, 9, 10, 11, 12, 13, 14, 15, 16, 17, 18, 19, 20, 21, 22, 23, 24, 25, 30, 35, 40, 45, 50, 55, 60, 65, 75, 80, 85, 90, 95, 100, or more amino acids in length. Such fragments preferably retain one or more of the biological activities of Sax2 protein and/or retain an immunological (antigenic) property of Sax2 protein. These fragments may be purified according to known methods, such as precipitation (e.g. ammonium sulfate), HPLC, ion exchange chromatography, affinity chromatography (including immunoaffinity chromatography) or various size separations (sedimentation, gel electrophoresis, gel filtration).

a. Structural Features of the Polypeptide.

The Sax2 gene encodes a protein having a molecular weight of 58 kDa. The Sax2 gene has a homoebox and an EH1 domain which is involved in repressing target genes. The Sax2 also has a predicted nuclear localization element due to its function as a transcription factor.

b. Functional Aspects.

The homeobox Sax2 gene is expressed early during embryogenesis mainly in the nervous system. Deletion of the Sax2 gene results in growth retardation and high postnatal lethality within the first 3 weeks. Mutant pups are born in a Mendelian distribution and indistinguishable from their littermates. However, by day 3, mutants display retarded growth. Although mutant pups are runted, they do not exhibit any abnormal behavior or motor skills. All mutant pups show normal suckling behavior and milk can be observed in their stomach (Simon and Lufkin, Mol. Cell. Biol. 23: 9046-9060, 2003). To further determine whether Sax2 null mutant mice are exhibiting gross behavioral or neurological differences the animals were exposed to tests of neurological reflexes, the jar-circling test for hyperactivity and adaptation to a new environment, the gait test for motor abilities and the forelimb grip test for neuromuscular abnormalities. In all cases, with the exception of the grip test Sax2 null mutants are indistinguishable from their wild-type littermates. The grip test revealed that Sax2 null mutants were able to grip the suspension bar but could not hold on as long as the wild-type pups (Simon and Lufkin, Mol. Cell. Biol. 23: 9046-9060, 2003). Histological preparations of muscle tissue did not present any abnormalities.

To further analyze the cause of growth retardation and premature death, different organs of wild-type and Sax2 null mutant pups at different days postpartum were examined and revealed a lack of WAT and reduction of BAT in the mutant starting at birth. Similar phenotypes have been reported for the Vgf gene, a neuropeptide expressed in the arcuate nucleus (Hahm et al, J. Neurosci. 22: 6929-6938, 1999) and the A-ZIP/F-1 gene, which is expressed in the adipocyte tissue and provides a good model for human lipoatrophy (Moitra et al, Genes & Dev 12: 3168-3181, 1998; Haluzik et al, Diabetes 51: 2113-2118, 2002). Unlike Vgf and A-ZIP/F-1, Sax2 expression does not occur either in the arcuate nucleus or in the adipocyte tissue but in the mid/hindbrain boundary and the ventral midbrain as well as the ventral neural tube suggesting an endocrine function for Sax2. Lack of adipocyte tissue can be caused by many pathologies including defects in adipocyte differentiation and maturation, cacchexia and/or defects in glucose/fat metabolism. It is known that homoebox genes play a crucial role in the regulation of many processes during embryogenesis and development. It is possible that Sax2 is involved in the regulation of transcription factors involved in adipocyte differentiation, e.g. interfere with factors involved in the differentiation process like C/EBPα, PPARγ and ADD-1/SREBP-1. However the data generated from the studies discussed herein showed that adipocytes are present in Sax2 null mutants, but unlike the wild-type cells do not contain any fat incorporation.

The lack of adipocyte tissue in the Sax2 null mutant is caused by deregulation of the glucose and/or fat metabolism or related pathways that are involved in the generation of energy storage components. Sax2 expression also occurs in the vicinity of serotonergic neurons in the dorsal raphe nucleus and its expression coincides with the onset of serotonergic neuron differentiation. As mentioned above serotonergic neurons play a critical role in food uptake and body weight. Hence, it is suggested that the Sax2 also may play a role in the differentiation of serotonergic neurons. Thus, there is evidence that Sax2 gene expression is required for fat deposition but not for adipocyte differentiation, and that Sax2 is an endocrine factor that is produced in the brain but has an effect on adipocyte tissue. These key findings allow an understanding of energy homeostasis and development of therapies which concentrate on decreasing the activity or expression of Sax2 gene. Such therapies and understanding of energy homeostasis allows for the treatment, amelioration or prevention of illnesses like lipoatrophy, obesity, diabetes and heart failures.

When the present application refers to the function of Sax2 protein or “wild-type” activity, it is meant that the molecule in question has the ability to regulate fat deposition in adipocytes. An assessment of the particular molecules that possess such activities may be achieved using standard assays familiar to those of skill in the art. For example, the histological studies such as those described in the Examples will readily reveal whether fat deposition in WAT or BAT is affected by a given Sax2-related molecule

In certain embodiments, Sax2 protein analogs and variants may be prepared and will be useful in a variety of applications. Amino acid sequence variants of the polypeptide can be substitutional, insertional or deletion variants. Deletion variants lack one or more residues of the native protein which are not essential for function or immunogenic activity. A common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, also called fusion proteins, are discussed below.

Substitutional variants typically exchange one amino acid of the wild type for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

A particular aspect of the present invention contemplates generating Sax2 protein mutants in which the homeodomain and/or the EH1 motif are deleted or modified (i.e., mutated). Such mutants will yield important information pertaining to the biological activity, physical structure and receptor or ligand binding potential of the Sax2 protein molecule. An alternative approach employs alanine scanning in which residues throughout molecule are randomly replaced with an alanine residue.

In order to construct such mutants, one of skill in the art may employ well known standard technologies. Specifically contemplated are N-terminal deletions, C-terminal deletions, internal deletions, as well as random and point mutagenesis.

N-terminal and C-terminal deletions are forms of deletion mutagenesis that take advantage for example, of the presence of a suitable single restriction site near the end of the C- or N-terminal region. The DNA is cleaved at the site and the cut ends are degraded by nucleases such as BAL31, exonuclease III, DNase I, and S1 nuclease. Rejoining the two ends produces a series of DNAs with deletions of varying size around the restriction site. Proteins expressed from such mutants can be assayed for appropriate apoptotic activity as described throughout the specification. Similar techniques may be employed for internal deletion mutants by using two suitably placed restriction sites, thereby allowing a precisely defined deletion to be made, and the ends to be religated as above.

Also contemplated are partial digestion mutants. In such instances, one of skill in the art would employ a “frequent cutter”, which cuts the DNA in numerous places depending on the length of reaction time. Thus, by varying the reaction conditions it will be possible to generate a series of mutants of varying size, which may then be screened for activity.

A random insertional mutation may also be performed by cutting the DNA sequence with a DNase I, for example, and inserting a stretch of nucleotides that encode, 3, 6, 9, 12 etc., amino acids and religating the end. Once such a mutation is made the mutants can be screened for various activities presented by the wild-type protein.

Point mutagenesis also may be employed to identify with particularity which amino acid residues are important in particular activities associated with Sax2 protein. Thus, one of skill in the art will be able to generate single base changes in the DNA strand to result in an altered codon and a missense mutation.

The amino acids of a particular protein can be altered to create an equivalent, or even an improved, second-generation molecule. Such alterations contemplate substitution of a given amino acid of the protein without appreciable loss of interactive binding capacity with structures such as, for example, antigen-binding regions of antibodies or binding sites on substrate molecules or receptors. Since it is the interactive capacity and nature of a protein that defines that protein's biological functional activity, certain amino acid substitutions can be made in a protein sequence, and its underlying DNA coding sequence, and nevertheless obtain a protein with like properties. Thus, various changes can be made in the DNA sequences of genes without appreciable loss of their biological utility or activity, as discussed below. Table 1 below shows the codons that encode particular amino acids.

In making such changes, the hydropathic index of amino acids may be considered. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics Kyte & Doolittle, J. Mol. Biol., 157(1):105-132, 1982, incorporated herein by reference). Generally, amino acids may be substituted by other amino acids that have a similar hydropathic index or score and still result in a protein with similar biological activity, i.e., still obtain a biological functionally equivalent protein.

In addition, the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As such, an amino acid can be substituted for another having a similar hydrophilicity value and still obtain a biologically equivalent and immunologically equivalent protein.

Exemplary amino acid substitutions that may be used in this context of the invention include but are not limited to exchanging arginine and lysine; glutamate and aspartate; serine and threonine; glutamine and asparagine; and valine, leucine and isoleucine. Other such substitutions that take into account the need for retention of some or all of the biological activity whilst altering the secondary structure of the protein will be well known to those of skill in the art.

Another type of variant that is specifically contemplated for the preparation of polypeptides according to the invention is the use of peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of Sax2 protein, but with altered and even improved characteristics.

Other mutants that are contemplated are those in which entire, domains of the Sax2 protein are switched with those of another related protein. Domain switching is well-known to those of skill in the art and is particularly useful in generating mutants having domains from related species. For example, Sax2 shows high similarity to the Drosophila S59/slouch and the murine Sax1 genes. In mammals there appear to be only two Sax genes forming the NK1 gene family which is part of the NKL gene cluster. Expression patterns for the NK1 gene family have been described in Drosophila, mouse and chicken. Domains from Drosophila S59/slouch, the murine Sax1 genes, or the NK1 gene family may be readily switched with domains from the Sax2 gene identified herein.

Domain switching involves the generation of chimeric molecules using different but related polypeptides. For example, by comparing the sequence of Sax2 protein with that of similar sequences from another source and with mutants and allelic variants of these polypeptides, one can make predictions as to the functionally significant regions of these molecules. It is possible, then, to switch related domains of these molecules in an effort to determine the criticality of these regions to Sax2 protein function. These molecules may have additional value in that these “chimeras” can be distinguished from natural molecules, while possibly providing the same or even enhanced function.

In addition to the mutations described above, the present invention further contemplates the generation of a specialized kind of insertional variant known as a fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. It is likely that the Sax2 protein product is a secreted endocrine, which has a receptor on WAT and/or BAT. Fusion to a polypeptide that can be used for purification of the receptor-Sax2 protein complex would serve to isolate the Sax2 receptor for identification and analysis. Similar identifications have successfully been performed with leptin and the leptin receptor.

There are various commercially available fusion protein expression systems that may be used in the present invention. Particularly useful systems include but are not limited to the glutathione S-transferase (GST) system (Pharmacia, Piscataway, N.J.), the maltose binding protein, system (NEB, Beverley, Mass.), the FLAG system (IBI, New Haven, Conn.), and the 6×His system (Qiagen, Chatsworth, Calif.). These systems are capable of producing recombinant polypeptides bearing only a small number of additional amino acids, which are unlikely to affect the antigenic ability of the recombinant polypeptide. For example, both the FLAG system and the 6×His system add only short sequences, both of which are known to be poorly antigenic and which do not adversely affect folding of the polypeptide to its native conformation. Another N terminal fusion that is contemplated to be useful is the fusion of a Met Lys dipeptide at the N terminal region of the protein or peptides. Such a fusion may produce beneficial increases in protein expression or activity.

A particularly useful fusion construct may be one in which a Sax2 protein or peptide is fused to a hapten to enhance immunogenicity of a Sax2 protein fusion construct. Such fusion constructs to increase immunogenicity are well known to those of skill in the art, for example, a fusion of Sax2 protein with a helper antigen such as hsp70 or peptide sequences such as from Diphtheria toxin chain or a cytokine such as IL-2 will be useful in eliciting an immune response. In other embodiments, fusion construct can be made which will enhance the targeting of the Sax2 protein-related compositions to a specific site or cell.

Other fusion constructs including a heterologous polypeptide with desired properties, e.g., an Ig constant region to prolong serum half life or an antibody or fragment thereof for targeting also are contemplated. Other fusion systems produce polypeptide hybrids where it is desirable to excise the fusion partner from the desired polypeptide. In one embodiment, the fusion partner is linked to the recombinant Sax2 protein polypeptide by a peptide sequence containing a specific recognition sequence for a protease. Examples of suitable sequences are those recognized by the Tobacco Etch Virus protease (Life Technologies, Gaithersburg, Md.) or Factor Xa (New England Biolabs, Beverley, Mass.).

It will be desirable to purify Sax2 protein or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure, peptide are ion-exchange chromatography, exclusion chromatography; polyacrylamide gel electrophoresis; isoelectric focusing; affinity columns specific for protein fusion moieties; affinity columns containing Sax2-specific antibodies. A particularly efficient method of purifying peptides is fast protein liquid; chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification, steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE. It will therefore be appreciated that under differing electrophoresis conditions, the apparent molecular weights of purified or partially purified expression products may vary.

In addition to the full length Sax2 protein described herein, smaller Sax2 protein-related peptides may be useful in various embodiments of the present invention. Such peptides or indeed even the full length protein, of the invention can also be synthesized in solution or on a solid support in accordance with conventional techniques. Various automatic synthesizers are commercially available and can be used in accordance with known protocols. See, for example, Stewart and Young, Solid Phase Peptide Synthesis, 2d. ed., Pierce Chemical Co., (1984); Tam et al., J. Am. Chem. Soc., 105:6442, (1983); Merrifield, Science, 232: 341-347, (1986); and Barany and Merrifield, The Peptides, Gross and Meienhofer, eds, Academic Press, New York; 1-284, (1979), each incorporated herein by reference. The Sax2 protein active protein or portions of the Sax2 protein, which correspond to the selected regions described herein, can be readily synthesized and then screened in screening assays designed to identify reactive peptides.

Alternatively, recombinant DNA technology may be employed wherein a nucleotide sequence which encodes a peptide of the invention is inserted into an expression vector, transformed or transfected into an appropriate host cell and cultivated under conditions suitable for expression as described herein below.

U.S. Pat. No. 4,554,101 (incorporated herein by reference) also teaches the identification and preparation of epitopes from primary amino acid sequences on the basis of hydrophilicity. Thus, one of skill in the art would be able to identify epitopes from within any amino acid sequence encoded by any of the DNA sequences disclosed herein.

As discussed herein below, the Sax2 proteins or peptides, may be useful as antigens for the immunization of animals relating to the production of antibodies. It is envisioned that either Sax2 protein, or portions thereof, may be coupled, bonded, bound, conjugated or chemically-linked to one or more agents via linkers, polylinkers or derivatized amino acids. This may be performed such that a bispecific or multivalent composition or vaccine is, produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyanin (KLH) or bovine serum albumin (BSA).

### Sax2-Related Nucleic Acids

The present invention also provides, in another embodiment, an isolated nucleic acid encoding Sax2 protein. The nucleic acid or gene for the murine protein molecule has been identified. Homology studies may now be readily performed to identify the related human gene. Preferred embodiments of the present invention are directed to nucleic acid constructs comprising a Sax2 of SEQ ID NO:1 (or preferably the related human gene SEQ ID NO:3), operably linked to a heterologous promoter The present invention is not limited in scope to the particular gene(s) identified herein, however, seeing as one of ordinary skill in the art could, using the nucleic acids corresponding to the Sax2 gene, readily identify related homologs in various other species (e.g., rat, rabbit, monkey, gibbon, chimp, ape, baboon, cow, pig, horse, sheep, cat and other species).

In addition, it should be clear that the present invention is not limited to the specific nucleic acids disclosed herein. As discussed below, a “Sax2 gene” may contain a variety of different nucleic acid bases and yet still produce a corresponding polypeptide that is functionally indistinguishable, and in some cases structurally, from the human gene disclosed herein. The term “Sax2 gene” may be used to refer to any nucleic acid that encodes a Sax2 protein, peptide or polypeptide and, as such, is intended to encompass both genomic DNA and cDNA.

Similarly, any reference to a nucleic acid should be read as encompassing a host cell containing that nucleic acid and, in some cases, capable of expressing the product of that nucleic acid. In addition to therapeutic considerations, cells expressing nucleic acids of the present invention may prove useful in the context of screening for agents that induce, repress, inhibit, augment, interfere with, block, abrogate, stimulate or enhance the function of Sax2 gene or protein product, its receptor or endogenous protein on which Sax2 has an effect.

a. Nucleic Acids Encoding Sax2.

The murine Sax2 gene is disclosed in SEQ ID NO:1; the human gene is provided in SEQ ID NO:3. Nucleic acids according to the present invention (which include genomic DNA, cDNA, mRNA, as well as recombinant and synthetic sequences and partially synthetic sequences) may encode an entire Sax2 protein, polypeptide, or allelic variant, a domain of Sax2 protein that expresses an activity of the wild-type Sax2, or any other fragment or variant of the Sax2 protein sequences set forth herein.

The nucleic acid may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that due to the redundancy of the genetic code, a given Sax2 gene from a given species may be represented by degenerate variants that have slightly different nucleic acid sequences but, nonetheless, encode the same protein (see Table 1 below).

As used in this application, the term “a nucleic acid encoding a Sax2 protein” refers to a nucleic acid molecule that has been, isolated from total cellular nucleic acid. In preferred embodiments, the invention concerns a nucleic acid sequence essentially as set forth in SEQ ID NO:1. The term “functionally equivalent codon” is used herein to refer to codons that encode the same amino acid, such as the six codons for arginine or serine (Table 1, below), and also refers to codons that encode biologically equivalent amino acids, as discussed in the following pages.

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAG GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Nucleotide sequences that have at least about 50%, usually at least about 60%, more usually about 70%, most usually about 80%, preferably at least about 90% and most preferably about 95% of nucleotides that are identical to the nucleotides of SEQ ID NO:1 (or related human sequence of SEQ ID NO:3) are nucleic acids encoding a Sax2 protein. Sequences that are essentially the same as those set forth in SEQ ID NO:1 (or related human sequence of SEQ ID NO:3) may also be functionally defined as sequences that are capable of hybridizing to a nucleic acid segment containing the complement of SEQ ID NO:1 (or related human sequence of SEQ ID NO:3) under standard conditions.

The DNA segments of the present invention include those encoding biologically functional equivalent Sax2 proteins and peptides as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through any means described herein or known to those of skill in the art.

b. Oligonucleotide Probes and Primers.

The present invention also encompasses DNA segments that are complementary, or essentially complementary, to the sequence set forth in SEQ ID NO:1 (murine) or SEQ ID NO:3 human). Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO:1 (or SEQ ID NO:3) under relatively stringent conditions such as those described herein. Such sequences may encode the entire-Sax2 protein or functional or non-functional fragments thereof.

Alternatively, the hybridizing segments may be shorter oligonucleotides. Sequences of about 17 bases long should occur only once in the human genome and, therefore, suffice to specify a unique target sequence. Nucleotide sequences of this size that specifically hybridize to SEQ ID NO:1 (murine) or SEQ ID NO:3 (human) are useful as probes or primers. As used herein, an oligonucleotide that “specifically hybridizes” to SEQ ID NO:1 (or SEQ ID NO:3) means that hybridization under suitably (e.g., high) stringent conditions allows discrimination of SEQ ID NO:1 (or SEQ ID NO:3) from other apoptotic genes. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, or 1000 bases and longer are contemplated as well. Such oligonucleotides will find use, for example, as probes in Southern and Northern blots and as primers in amplification reactions.

Suitable hybridization conditions will be well known to those of skill in the art. In certain applications, it is appreciated that lower stringency conditions may be required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C. Formamide and SDS also may be used to alter the hybridization conditions.

One method of using probes and primers of the present invention is in the search for genes related to Sax2 sequences, more particularly, homologs of the Sax2 from other, species. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids also are routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

Of course site-directed mutagenesis is not the only method of generating potentially useful mutant Sax2 species and as such is not meant to be limiting. The present invention also contemplates other methods of achieving mutagenesis such as for example, treating the recombinant vectors carrying the gene of interest mutagenic agents, such as hydroxylamine, to obtain sequence variants.

c. Inhibitory Nucleic Acid Constructs.

As discussed herein, inhibition of Sax2 expression lead to a decrease in fat accumulation in adipose tissue. Thus, it is suggested that this secreted product is integrally involved in the energy homeostasis in adipose tissue. It would be advantageous to disrupt the activity or expression of Sax2 in indications where it is desirable to reduce the fat accumulation in adipose tissue. Such disruption may be achieved using a variety of methods known to those of skill in the art. The present section discusses nucleic acid-based methods of disrupting Sax2 expression. For example, the nucleic acid-based techniques may be used to block the expression of Sax2, and therefore, to perturb the deposition of fat into adipocytes. Polynucleotide products which are useful in this endeavor include antisense polynucleotides, ribozymes, RNAi, and triple helix polynucleotides that modulate the expression of Sax2.

Antisense polynucleotides and ribozymes are well known to those of skill in the art. Crooke and B. Lebleu, eds. Antisense Research and Applications (1993) CRC Press; and Antisense RNA and DNA (1988) D. A. Melton, Ed. Cold Spring Harbor Laboratory Cold Spring Harbor, N.Y. Anti-sense RNA and DNA molecules act to directly block the translation of mRNA by binding to targeted mRNA and preventing protein translation. An example of an antisense polynucleotide is an oligodeoxyribonucleotide derived from the translation initiation site, e.g., between −10 and +10 regions of the relevant nucleotide sequence.

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo; such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozymes) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction, sites for the remaining portion of the construct and, therefore, would be used for the rest of the sequence.

As indicated above, the murine DNA and protein sequences for Sax2 are provided in SEQ ID NO:1 and SEQ ID NO:2, respectively, and the human predicted sequences are presented in SEQ ID NO:3 and SEQ ID NO:10, respectively. Related Sax2 protein and/or nucleic acid sequences from other sources may be identified using probes directed at the sequences of SEQ ID.NO:1 (murine) or SEQ ID NO:3 (human). Such additional sequences may be useful in certain aspects of the present invention. Although antisense sequences may be full length genomic or cDNA copies, they also may be shorter fragments or oligonucleotides e.g., polynucleotides of 100 or less bases. Although shorter oligomers (8-20) are easier to make and more easily permeable in vivo, other factors also are involved in determining the specificity of base pairing. For example, the binding affinity and sequence specificity of an oligonucleotide to its complementary target increases with increasing length. It is contemplated that oligonucleotides of 8, 9, 10, 11, 12, 13, 14, 15, 16, 17, 18, 19, 20, 25, 30, 35, 40, 45, 50, or more base pairs will be used.

Ribozymes are enzymatic RNA molecules capable of catalyzing the specific cleavage of RNA. The mechanism of ribozyme action involves sequence specific interaction of the ribozyme molecule to complementary target RNA, followed by an endonucleolytic cleavage. Within the scope of the invention are engineered hammerhead or other motif ribozyme molecules that specifically and efficiently catalyze endonucleolytic cleavage of RNA sequences encoding protein complex components.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate targets may also be evaluated by testing their accessibility to hybridization with complementary oligonucleotides, using ribonuclease protection assays. See, Draper PCT WO 93/23569; and U.S. Pat. No. 5,093,246.

Nucleic acid molecules used in triple helix formation for the inhibition of transcription are generally single stranded and composed of deoxyribonucleotides. The base composition must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single-strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

Another technique that is of note for reducing or disrupting the expression of a gene is RNA interference (RNAi), also known as small interfering RNA (siRNA). The term “RNA interference” was first used by researchers studying C. elegans and describes a technique by which post-transcriptional gene silencing (PTGS) is induced by the direct introduction of double stranded RNA (dsRNA: a mixture of both sense and antisense strands). Injection of dsRNA into C. elegans resulted in much more efficient silencing than injection of either the sense or the antisense strands alone (Fire et al, Nature 391:806-811, 1998). Just a few molecules of dsRNA per cell is sufficient to completely silence the expression of the homologous gene. Furthermore, injection of dsRNA caused gene silencing in the first generation offspring of the C. elegans indicating that the gene silencing is inheritable (Fire et al., Nature 391:806-811, 1998). Current models of PTGS indicate that short stretches of interfering dsRNAs (21-23 nucleotides; siRNA also known as “guide RNAs”) mediate PTGS. siRNAs are apparently produced by cleavage of dsRNA introduced directly or via a transgene or virus. These siRNAs may be amplified by an RNA-dependent RNA polymerase (RdRP) and are incorporated into the RNA-induced silencing complex (RISC), guiding the complex to the homologous endogenous mRNA, where the complex cleaves the transcript. Thus, siRNAs are nucleotides of a short length (typically 18-25 bases, preferably 19-23 bases in length) which incorporate into an RNA-induced silencing complex in order to guide the complex to homologous endogenous mRNA for cleavage and degradation of the transcript.

While most of the initial studies were performed in C. elegans, RNAi is gaining increasing recognition as a technique that may be used in mammalian cell. It is contemplated that RNAi, or gene silencing, will be particularly useful in the disruption of Sax2 expression, and this may be achieved in a tissue-specific manner where desired. By placing a gene fragment encoding the desired dsRNA behind an inducible or tissue-specific promoter, it should be possible to inactivate genes at a particular location within an organism or during a particular stage of development.

Variations on RNA interference (RNAi) technology is revolutionizing many approaches to experimental biology, complementing traditional genetic technologies, mimicking the effects of mutations in both cell cultures and in living animals. (McManus & Sharp, Nat. Rev. Genet. 3, 737-747 (2002)). RNAi has been used to elicit gene-specific silencing in cultured mammalian cells using 21-nucleotide siRNA duplexes (Elbashir et al., Nature, 411:494-498, 2001; Fire et al., Nature 391, 199-213 (1998), Hannon, G. J., Nature 418, 244-251 (2002))). In the same cultured cell systems, transfection of longer stretches of dsRNA yielded considerable nonspecific silencing. Thus, RNAi has been demonstrated to be a feasible technique for use in mammalian cells and could be used for assessing gene function in cultured cells and mammalian systems, as well as for development of gene-specific therapeutics. In particularly preferred embodiments, the siRNA molecule is between 20 and 25 oligonucleotides in length an is derived from the sequence of SEQ ID NO:1. Particularly preferred siRNA molecules are 21-23 bases in length.

Anti-sense RNA and DNA molecules, ribozymes, RNAi and triple helix molecules can be prepared by any method known in the art for the synthesis of DNA and RNA molecules. These include techniques for chemically synthesizing oligodeoxyribonucleotides well known in the art including, but not limited to, solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably or transiently into cells.

Commercial providers such as Ambion Inc. (Austin; TX), Darmacon Inc. (Lafayette, Colo.), InvivoGen (San Diego, Calif.), and Molecula Research Laboratories, LLC (Herndon, Va.) generate custom siRNA molecules. In addition, commercial-kits are available to produce custom siRNA molecules, such as SILENCER™ siRNA Construction Kit (Ambion Inc., Austin, Tex.) or psiRNA System (InvivoGen, San Diego, Calif.). These siRNA molecules may be introduced into cells through transient transfection or by introduction of expression vectors that continually express the siRNA in transient or stably transfected mammalian cells. Transfection may be accomplished by well known methods including methods such as infection, calcium chloride, ejectroporation, microinjection, lipofection or the DEAE-dextran method or other known techniques. These techniques are well known to those of skill in the art.

### Recombinant Protein Production.

Given the above disclosure of the Sax2 gene, it is possible to produce Sax2 protein by recombinant techniques. A variety of expression vector/host systems may be utilized to contain and express a Sax2 protein coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells that are useful in recombinant protein production include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of Sax2 protein in bacteria, yeast and other invertebrates are described herein below.

The DNA sequence encoding the mature form of the protein is amplified by PCR and cloned into an appropriate vector for example, pGEX 3× (Pharmacia, Piscataway, N.J.). The pGEX vector is designed to produce a fusion protein comprising glutathione S transferase (GST), encoded by the vector, and a protein encoded by a DNA fragment inserted into the vector's cloning site. The primers for the PCR may be generated to include for example, an appropriate cleavage site.

Treatment of the recombinant fusion protein with thrombin or factor Xa (Pharmacia, Piscataway, N.J.) is expected to cleave the fusion protein, releasing the proapoptotic factor from the GST portion. The pGEX 3×/Sax2 protein construct is transformed into E. coli XL 1 Blue cells (Stratagene, La Jolla Calif.), and individual transformants were isolated and grown. Plasmid DNA from individual transformants is purified and partially sequenced using an automated sequencer to confirm the presence of the desired Sax2 protein-encoding gene insert in the proper-orientation.

Knowledge of Sax2 gene sequences allows for modification of cells to permit or increase expression of endogenous Sax2. The cells can be modified (heterologous promoter is inserted in such a manner that it is operably linked to, e.g., by homologous recombination) to provide increase Sax2 expression by replacing, in whole or in part the naturally occurring promoter with all or part of a heterologous promoter so that the cells express Sax2 protein at higher levels. The heterologous promoter is inserted in such a manner that it is operably linked to Sax2 gene-sequences. (e.g., PCT International Publication No. WO96/12650; PCT International Publication No. WO 92/20808 and PCT International Publication No. WO 91/09955). It is contemplated that, in addition to the heterologous promoter DNA, amplifiable marker DNA (e.g., ada, dhfr and the multifunctional CAD gene which encodes carbamyl phosphate synthase, aspartate transcarbamylase and dihydroorotase) and/or intron DNA may be inserted along with the heterologous promoter DNA. If linked to the Sax2 gene sequence, amplification of the marker DNA by standard selection, methods results in co amplification of the Sax2 gene sequences in the cells.

While certain embodiments of the present invention contemplate producing the Sax2 protein using synthetic peptide synthesizers and subsequent FPLC analysis and appropriate refolding of the cysteine double bonds, it is contemplated that recombinant protein production also may be used to produce the Sax2 protein compositions. For example, induction of the GST/x2 fusion protein is achieved by growing the transformed XL 1 Blue culture at 37° C. in LB medium (supplemented with carbenicillin) to an optical density at wavelength 600 nm of 0.4, followed by further incubation for 4 hours in the presence of 0.5 mM Isopropyl β-D Thiogalactopyranoside (Sigma Chemical Co., St. Louis Mo.).

The fusion protein, expected to be produced as an insoluble inclusion body in the bacteria, may be purified as follows. Cells are harvested by centrifugation; washed in 0.15 M NaCl, 10 mM Tris, pH 8, 1 mM EDTA; and treated with 0.1 mg/ml lysozyme (Sigma Chemical Co.) for 15 minutes at room temperature. The lysate is cleared by sonication, and cell debris is pelleted by centrifugation for 10 minutes at 12,000×g. The fusion protein containing pellet is resuspended in 50 mM Tris, pH 8, and 10 mM EDTA, layered over 50% glycerol, and centrifuged for 30 min. at 6000×g. The pellet is resuspended in standard phosphate buffered saline solution (BS) free of Mg++ and Ca++. The fusion protein is further purified by fractionating the resuspended pellet in a denaturing SDS polyacrylamide gel. The gel is soaked in 0.4 M KCl to visualize the protein, which is excised and electroeluted in gel running buffer lacking SDS. If the GST/Sax2 protein is produced in bacteria as a soluble protein, it may be purified using the GST Purification Module (Pharmacia Biotech).

The fusion protein may be subjected to thrombin digestion to cleave the GST from the mature Sax2 protein. The digestion reaction (20-40 μg fusion protein, 20-30 units human thrombin (4000 U/mg (Sigma) in 0.5 ml PBS) is incubated 16-48 hrs at room temperature and loaded on a denaturing SDS PAGE gel to fractionate the reaction products. The gel is soaked in 0.4 M KCl to visualize the protein bands. The identity of the protein band corresponding to the expected molecular weight of Sax2 protein may be confirmed by partial amino acid sequence analysis using an automated sequencer (Applied Biosystems Model 473A, Foster City, Calif.).

Alternatively, the DNA sequence encoding the predicted mature Sax2 protein may be cloned into a plasmid containing a desired promoter and, optionally, a leader sequence (see, e.g., Better et al., Science, 240: 1041 43, 1988). The sequence of this construct may be confirmed by automated sequencing. The plasmid is then transformed into E. coli strain MC1061 using standard procedures employing CaCl2 incubation and heat shock treatment of the bacteria (Sambrook et al., supra). The transformed bacteria are grown in LB medium supplemented with carbenicillin, and production of the expressed protein is induced by growth in a suitable medium. If present, the leader sequence will effect secretion of the mature Sax2 protein and be cleaved during secretion.

The secreted recombinant protein is purified from the bacterial culture media by standard protein purification techniques well known to those of skill in the art.

Similarly, a yeast system may be employed to generate the recombinant peptide. This may be performed using standard commercially available expression systems, e.g., the Pichia Expression System (Invitrogen, San Diego, Calif.), following the manufacturer's instructions. This system relies on the pre pro alpha sequence to direct secretion, and transcription of the insert is driven by the alcohol oxidase (AOX1) promoter upon induction by methanol. The secreted recombinant protein is purified from the yeast growth medium by standard protein purification methods.

Alternatively, the cDNA encoding Sax2 protein may be cloned into the baculovirus expression vector pVL1393 (PharMingen, San Diego, Calif.). This vector is then used according to the manufacturer's directions (PharMingen) to infect Spodoptera frugiperda cells in sF9 protein free media and to produce recombinant protein. The protein is purified and concentrated from the media using a heparin Sepharose column (Pharmacia, Piscataway, N.J.) and sequential molecular sizing columns (Amicon, Beverly, Mass.), and resuspended in PBS. SDS PAGE analysis shows a single band and confirms the size of the protein, and Edman sequencing on a Porton 2090 Peptide Sequencer confirms its N terminal sequence.

Alternatively, the Sax2 may be expressed in an insect system. Insect systems for protein expression are well known to those of skill in the art. In one such system, Autographa californica nuclear polyhedrosis virus (AcNPV) is used as a vector to express foreign genes in Spodoptera frugiperda cells or in Trichoplusia larvae. The Sax2 gene sequence is cloned into a nonessential region of the virus, such as the polyhedrin gene, and placed under control of the polyhedrin promoter. Successful insertion of sequence will render the polyhedrin gene inactive and produce recombinant virus lacking coat protein coat. The recombinant viruses are then used to infect S. frugiperda cells or Trichoplusia larvae in which Sax2 is expressed (Smith et al., J Virol 46: 584, 1983; Engelhard E K et al., Proc Nat Acad Sci 91: 3224-7, 1994).

Mammalian host systems for the expression of the recombinant protein also are well known to those of skill in the art. Host cell strains may be chosen for a particular ability to process the expressed protein or produce certain post translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

It is preferable that the transformed cells are used for long-term, high-yield protein production and as such stable expression is desirable. Once such cells are transformed with vectors that contain selectable markers along with the desired expression cassette, the cells may be allowed to grow for 1-2 days in an enriched media before they are switched to selective media. The selectable marker is designed to confer resistance to selection and its presence allows growth and recovery of cells which successfully express the introduced sequences. Resistant clumps of stably transformed cells can be proliferated using tissue culture techniques appropriate to the cell.

A number of selection systems may be used to recover the cells that have been transformed for recombinant protein production. Such selection systems include, but are not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to methotrexate; gpt, that confers resistance to mycophenolic acid; neo, that confers resistance to the aminoglycoside G418; als which confers resistance to chlorsulfuron; and hygro, that confers resistance to hygromycin. Additional selectable genes that may be useful include trpB, which allows cells to utilize indole in place of tryptophan, or hisD, which allows cells to utilize histinol in place of histidine. Markers that give a visual indication for identification of transformants include anthocyanins, glucuronidase and its substrate, GUS, and luciferase and its substrate, luciferin.

### Vectors for Cloning, Gene Transfer and Expression

As discussed in the previous section, expression vectors are employed to express the Sax2, which can then be purified and, for example, be used to vaccinate animals to generate antisera or monoclonal antibody with which further studies may be conducted. In other embodiments, expression vectors may be used in gene therapy applications to introduce Sax2 protein encoding nucleic acids into cells in need thereof and/or to induce Sax2 protein expression in such cells. The present section is directed to a description of the production of such expression vectors.

Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products also are provided, as it an element that links expression of the drug selection markers to expression of the polypeptide.

a. Regulatory Elements.

Promoters and Enhancers. Throughout this application, the term “expression construct” or “expression vector” is meant to include any type of genetic construct containing a nucleic acid coding for gene products in which part or all of the nucleic acid encoding sequence is capable of being transcribed. The transcript may be translated into a protein; but it need not be. In certain embodiments, expression includes both transcription of a gene and translation of mRNA into a gene product.

The nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Promoters are composed of discrete functional modules, each consisting of approximately 7-20 bp of DNA, and containing one or more recognition sites for transcriptional activator or repressor proteins.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter, the phosphoglycerol kinase promoter and glyceraldehyde-3-phosphate dehydrogenase promoter, all of which are promoters well known and readily available to those of skill in the art, can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well known properties, the level and pattern of expression of the protein of interest following transfection or transformation can be optimized.

Inducible promoter systems may be used in the present invention, e.g., inducible ecdysone system (Invitrogen, Carlsbad, Calif.), which is designed to allow regulated expression of a gene of interest in mammalian cells. Another inducible system that would be useful is the Tet-Off™ or Tet-On™ system (Clontech, Palo Alto, Calif.) originally developed by Gossen and Bujard (Gossen and Bujard, Proc Natl Acad Sci U S A. 15; 89(12):5547 51, 1992; Gossen et al., Science, 268(5218):1766 9, 1995).

In some circumstances, it may be desirable to regulate expression of a transgene in a gene therapy vector. For example, different viral promoters with varying strengths of activity may be utilized depending on the level of expression desired. In mammalian cells, the CMV immediate early promoter is often used to provide strong transcriptional activation. Modified versions of the CMV promoter that are less potent have also been used when reduced levels of expression of the transgene are desired. When expression of a transgene in hematopoietic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate.

In certain indications, it may be desirable to activate transcription at specific times after administration of the gene therapy vector. This may be done with such promoters as those that are hormone, or cytokine regulatable. For example in gene therapy applications where the indication is a gonadal tissue where specific steroids are produced or routed to, use of androgen or estrogen regulated promoters may be advantageous. Such promoters that are hormone regulatable include MMTV, MT-1, ecdysone and RuBisco. Other hormone regulated promoters such as those responsive to thyroid, pituitary and adrenal hormones are expected to be useful in the present invention. Cytokine and inflammatory protein responsive promoters that could be used include. K and T Kininogen. (Kageyama et al., J Biol Chem. 262(5):2345 51, 1987), c-fos, TNF-alpha, C-reactive protein (Arcone et al., Nucleic Acids Res. 16(8):3195 207, 1988), haptoglobin (Oliviero et al., EMBO J. 6(7):1905.12, 1987), serum amyloid A2, C/EBP alpha, IL-1, IL-6 (Poli and Cortese, Proc Natl Acad Sci USA. 86(21):8202 6, 1989), Complement C3 (Wilson et al., Mol Cell Biol. 10(12):6181 91, 1990), IL-8, alpha-1 acid glycoprotein (Prowse and Baumann, Mol Cell Biol. 8(1):42 51, 1988), alpha-1 antitypsin, lipoprotein lipase (Zechner et al., Mol Cell Biol, 8(6):2394 401, 1988), angiotensinogen (Ron et al., Mol Cell Biol. 11(5):2887 95, 1991), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 antichymotrypsin.

It is envisioned that cell cycle regulatable promoters may be useful in the present invention. For example, in a bicistronic gene therapy vector, use of a strong CMV promoter to drive expression of a first gene such as p16 that arrests cells in the G1 phase could be followed by expression of a second gene such as p53 under the control of a promoter that is active in the G1 phase of the cell cycle, thus providing a “second hit” that would push the cell into apoptosis. Other promoters such as those of various cyclins, PCNA, galectin-3, E2F1, p53 and BRCA1 could be used.

It is envisioned that any of the above promoters alone or in combination with another may be useful according to the present invention depending-on the action desired. In addition, this list of promoters should not be construed to be exhaustive or limiting, and those of skill in the art will know of other promoters that may be used in conjunction with the promoters and methods disclosed herein.

Another regulatory element contemplated for use in the present invention is an enhancer. These are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at, a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization. Enhancers useful in the present invention are well known to those of skill in the art and will depend on the particular expression system being employed (Scharf D et al. Results Probl Cell Differ 20: 125-62, 1994; Bittner et al Methods in Enzymol 153: 516-544, 1987).

Polyadenylation Signals. Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

IRES. In certain embodiments of the invention, the use of internal ribosome entry site (IRES) elements is contemplated to create multigene, or polycistronic, messages. IRES elements are able to bypass the ribosome scanning model of 5′ methylated Cap dependent translation and begin translation at internal sites (Pelletier and Sonenberg, Nature, 334:320-325, 1988). IRES elements from two members of the picornavirus family (poliovirus and encephalomyocarditis) have been described (Pelletier and Sonenberg, 1988 supra), as well an IRES from a mammalian message (Macejak and Sarnow, Nature, 353:90-94, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

b. Delivery of Expression Vectors.

There are a number of ways in which expression constructs may introduced into cells. In certain embodiments of the invention, the expression construct comprises a virus or engineered construct derived from a viral genome. In other embodiments, non-viral delivery is contemplated. The ability of certain viruses to enter cells via receptor-mediated endocytosis, to integrate into host cell genome and express viral genes stably and efficiently have made them attractive candidates for the transfer of foreign genes into mammalian cells (Ridgeway, In: Rodriguez. R L, Denhardt D T, ed. Vectors: A survey of molecular cloning vectors and their uses. Stoneham: Butterworth, 467 492, 1988; Nicolas and Rubenstein, In: Vectors: A survey of molecular cloning vectors and their uses, Rodriguez & Denhardt (eds.), Stoneham: Butterworth, 493 513, 1988; Baichwal and Sugden, In: Gene Transfer, Kucherlapati R, ed., New York, Plenum Press, 117 148, 1986; Temin, In: gene Transfer, Kucherlapati (ed.), New York: Plenum Press, 149 188, 1986). The first viruses used as gene vectors were DNA viruses including the papovaviruses (simian virus 40, bovine papilloma virus, and polyoma) (Ridgeway, 1988 supra; Baichwal and Sugden, 1986 supra) and adenoviruses (Ridgeway, 1988 supra; Baichwal and Sugden, 1986 supra). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988 supra; Temin, 1986 supra).

It is now widely recognized that DNA may be introduced into a cell using a variety of viral vectors. In such embodiments, expression constructs comprising viral vectors containing the genes of interest may be adenoviral (see for example, U.S. Pat. No. 5,824,544; U.S. Pat. No. 5,707,618; U.S. Pat. No. 5,693,509; U.S. Pat. No. 5,670,488; U.S. Pat. No. 5,585,362; each incorporated herein by reference), retroviral (see for example, U.S. Pat. No. 5,888,502; U.S. Pat. No. 5,830,725; U.S. Pat. No. 5,770,414; U.S. Pat. No. 5,686,278; U.S. Pat. No. 4,861,719 each incorporated herein by reference), adeno-associated viral (see for example, U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479 each incorporated herein by reference), an adenoviral-adenoassociated viral hybrid (see for example, U.S. Pat. No. 5,856,152 incorporated herein by reference) or a vaccinia viral or a herpesviral (see for example, U.S. Pat. No. 5,879,934; U.S. Pat. No. 5,849,571; U.S. Pat. No. 5,830,727; U.S. Pat. No. 5,661,033; U.S. Pat. No. 5,328,688 each incorporated herein by reference) vector.

There are a number of alternatives to viral transfer of genetic constructs. This section provides a discussion of methods and compositions of non-viral gene transfer. DNA constructs of the present invention are generally delivered to a cell, and in certain situations, the nucleic acid or the protein to be transferred may be transferred using non-viral methods.

Several non-viral methods for the transfer of expression constructs into cultured mammalian cells are contemplated by the present invention. These include calcium phosphate precipitation (Graham and Van Der Eb, Virology, 52:456-467, 1973; Chen and Okayama, Mol. Cell Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat. Acad. Sci. USA, 81:7161-7165, 1984), direct microinjection garland and Weintraub, J. Cell Biol., 101:1094-1099, 1985.), DNA-loaded liposomes (Nicolau and Sene, Biochim. Biophys. Acta, 721:185-190, 1982; Fraley et al., Proc. Natl. Acad. Sci. USA, 76:3348-3352, 1979; Felgner, Sci Am. 276(6):102 6, 1997; Felgner, Hum Gene Ther. 7(15):17913, 1996), cell sonication (Fechheimer et al., Proc. Natl. Acad. Sci. USA, 84:8463-8467, 1987), gene bombardment using high velocity microprojectiles (Yang et al., Proc. Natl. Acad. Sci. USA, 87:9568-9572, 1990), and receptor-mediated transfection (Wu and Wu, J. Biol. Chem., 262:4429-4432, 1987; Wu and Wu, Biochemistry, 27:887-892, 1988; Wu and Wu, Adv. Drug Delivery Rev., 12:159-167, 1993).

Once the construct has been delivered into the cell, the nucleic acid encoding the therapeutic gene may be positioned and expressed at different sites. In certain embodiments, the nucleic acid encoding the therapeutic gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In a particular embodiment of the invention, the expression construct may be entrapped in a liposome. The addition of DNA to cationic liposomes causes a topological transition from liposomes to optically birefringent liquid-crystalline condensed globules (Radler et al., Science, 275(5301):810 4, 1997). These DNA-lipid complexes are potential non-viral vectors for use in gene therapy and delivery. Liposome-mediated nucleic acid delivery and expression of foreign DNA in vitro has been very successful. Also contemplated in the present invention are various commercial approaches involving “lipofection” technology. Complexing the liposome with a hemagglutinating virus (HVJ) may facilitate fusion with the cell membrane and promote cell entry of liposome-encapsulated DNA (Kaneda et al., Science, 243:375-378, 1989). In other embodiments, the liposome may be complexed or employed in conjunction with nuclear nonhistone chromosomal proteins (HMG-1) (Kato et al., J. Biol. Chem., 266:3361-3364, 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems which can be employed to deliver a nucleic acid encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin (Wagner et al., Proc. Natl. Acad. Sci. USA, 87(9):3410-3414, 1990). Recently, a synthetic neoglycoprotein, which recognizes the same receptor as ASOR, has been used as a gene delivery vehicle (Ferkol et al., FASEB J., 7:1081-1091, 1993; Perales et al., Proc. Natl. Acad. Sci., USA 91:4086-4090, 1994) and epidermal growth factor (EGF) has also been used to deliver genes to squamous carcinoma cells (Myers, EPO 0273085).

In other embodiments, the delivery vehicle may comprise a ligand and a liposome. Thus, it is feasible that a nucleic acid encoding a therapeutic gene also may be specifically delivered into a particular cell type by any number of receptor-ligand systems with or without liposomes.

In another embodiment of the invention, the expression construct may simply consist of naked recombinant DNA or plasmids. Transfer of the construct may be performed by any of the methods mentioned above which physically or chemically permeabilize the cell membrane. This is applicable particularly for transfer in vitro, however, it may be applied for in vivo use as well. Dubensky et al. (Proc. Nat. Acad. Sci. USA, 81:7529-7533, 1984; Benvenisty and Neshif (Proc. Nat. Acad. Sci. USA, 83:9551-9555, 1986).

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., Nature, 327:70-73, 1987). Several devices for accelerating small particles have been developed. One such device relies on a high voltage discharge to generate an electrical current, which in turn provides the motive force (Yang et al., Proc. Natl. Acad. Sci. USA, 87:9568-9572, 1990). The microprojectiles used have consisted of biologically inert substances such as tungsten or gold beads.

Antibodies Immunoreactive with Sax2 Protein

In another aspect, the present invention contemplates an antibody that is immunoreactive with a Sax2 protein molecule of the present invention, or any portion thereof. Such antibodies include, but are not limited to, polyclonal, monoclonal, chimeric, single chain, Fab fragments and fragments produced by a Fab expression library, bifunctional/bispecific antibodies, humanized antibodies, CDR grafted antibodies, human antibodies and antibodies which include portions of CDR sequences specific for Sax2 protein.

Neutralizing antibodies, i.e., those which inhibit the activity of Sax2, may be especially preferred for therapeutic embodiments. In a preferred embodiment, an antibody is a monoclonal antibody. The invention provides for a pharmaceutical composition comprising a therapeutically effective amount of an antibody directed against Sax2 protein. The antibody may bind to and neutralize the apoptotic effects of the Sax2 protein. The antibody may be formulated with a pharmaceutically acceptable adjuvant. Means for preparing and characterizing antibodies are well known in the art (see, e.g., Harlow and Lane, ANTIBODIES: A LABORATORY MANUAL, Cold Spring Harbor Laboratory, Cold Spring Harbor, N.Y., 1988).

Briefly, a polygonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, goat, sheep, pigs or horses. Because of the relatively large blood volume of rabbits, a rabbit is a preferred choice for production of polyclonal antibodies.

Depending on the host species, various adjuvants may be used to increase immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

Antibodies, both polyclonal and monoclonal, specific for isoforms of antigen may be prepared using conventional immunization techniques, as will be generally known to those of skill in the art. As used herein, the term “specific for” is intended to mean that the variable regions of the antibodies recognize and bind Sax2 protein and are capable of distinguishing Sax2 protein from other antigens, for example other secreted proapoptotic factors. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

Monoclonal antibodies to Sax2 protein may be prepared using any technique which provides for the production of antibody molecules by continuous cell lines in culture. These include but are not limited to the hybridoma technique originally described by Koehler and Milstein (Nature 256: 495-497, 1975), the human B-cell hybridoma technique (Kosbor et al., Immunol Today 4:72, 1983; Cote et al., Proc Natl Acad Sci 80: 2026-2030, 1983) and the EBV-hybridoma technique (Cole et al., Monoclonal Antibodies and Cancer Therapy, Alan R Liss Inc, New York N.Y., pp 77-96, (1985).

When the hybridoma technique is employed, myeloma cell lines may be used. Such cell lines suited for use in hybridoma-producing fusion procedures preferably are non-antibody-producing, have high fusion efficiency, and enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions. It should be noted that the hybridomas and cell lines produced by such techniques for producing the monoclonal antibodies are contemplated to be novel compositions of the present invention.

In addition to the production of monoclonal antibodies, techniques developed for the production of “chimeric antibodies”, the splicing of mouse antibody genes to human antibody genes to obtain a molecule with appropriate antigen specificity and biological activity can be used (Morrison et al., Proc Natl Acad Sci 81: 6851-6855, 1984; Neuberger et al., Nature 312: 604-608, 1984; Takeda et al., Nature 314: 452-454; 1985). Alternatively, techniques described for the production of single chain antibodies (U.S. Pat. No. 4,946,778) can be adapted to produce Sax2 protein-specific single chain antibodies.

Antibodies may also be produced by inducing in vivo production in the lymphocyte population or by screening recombinant immunoglobulin libraries or panels of highly specific binding reagents as disclosed in Orlandi et al (Proc Natl Acad Sci 86: 3833-3837; 1989), and Winter G and Milstein C (Nature 349: 293-299, 1991).

It is proposed that the antibodies of the present invention will find useful application in standard immunochemical procedures, such as ELISA and Western blot methods and in immunohistochemical procedures such as tissue staining, as well as in other procedures which may utilize antibodies specific to Sax2 protein-related antigen epitopes. Additionally, it is proposed that monoclonal antibodies specific to the particular Sax2 protein of different species may be utilized in other useful applications.

In general, both polyclonal and monoclonal antibodies against Sax2 protein may be used in a variety of embodiments. In certain aspects, the antibodies may be employed for therapeutic purposes in which the inhibition of Sax2 protein activity is desired (e.g., to reduce fat deposition in adipocytes cells). Antibodies may be used to block Sax2 protein-action. In doing so, these antibodies can be used to ameliorate Sax2 mediated energy homeostasis, thereby reducing obesity-related disorders.

Antibodies of the present invention also may prove useful in diagnostic purposes in order, for example, to detect increases or decreases in Sax2 protein in tissue samples including adipocyte tissues, or fluid samples including blood serum, plasma and exudate samples. Additional aspects will employ the antibodies of the present invention in antibody cloning protocols to obtain cDNAs or genes encoding other Sax2 protein. They may also be used in inhibition studies to analyze the effects of Sax2 related peptides in cells or animals. Anti-Sax2 antibodies will also be useful in immunolocalization studies to analyze the distribution of Sax2 protein during various cellular events, for example, to determine the cellular or tissue-specific distribution of Sax2 protein polypeptides under different points in the cell cycle. A particularly useful application of such antibodies is in purifying native or recombinant Sax2 protein for example, using an antibody affinity column. The operation of all such immunological techniques will be known to those of skill in the art in light of the present disclosure.

### Therapeutic Methods

The present invention deals with the treatment of diseases that result from the increased expression of Sax2 protein. In one embodiment, this protein is seen secreted from mid/hindbrain boundary and the ventral midbrain as well as the ventral neural tube. The secreted product promotes fat deposition into adipose tissue. The gene is taught herein to regulate energy homeostasis in WAT and BAT. Regulation of this gene has significant implications in the treatment of obesity and obesity related disorders. It is contemplated that the Sax2 gene may be used in therapies in a similar manner to the uses proposed for leptin.

Hence, compositions designed to inhibit the expression or overexpression of Sax2 protein will be useful in treating or preventing obesity and obesity-related disorders, such as obesity-related diabetes, heart disease, stroke, cancer (such as colon cancer, endometrial cancer, and postmenopausal breast cancer), gallbladder disease, sleep apnea (interrupted breathing during sleep), osteoarthritis (wearing away of the joints). Those of skill in the art are aware of methods of such disorders. In preferred embodiments, the therapies of the invention are contemplated for the treatment of diabetes. Such therapies will generally involve inhibition of Sax2 expression. The therapeutic compositions can also comprise one or more additional agents effective in the treatment of the obesity or obesity-related disorder, e.g., treatment using insulin for diabetes. Other compositions which inhibit the expression, activity or function of Sax2 protein (e.g., antagonists) also are contemplated for use in such treatment methods.

Purified nucleic acid sequences, antisense molecules, PNAs, purified protein, antibodies, antagonists or inhibitors directed against Sax2 can all be used as pharmaceutical compositions. Delivery of these molecules for therapeutic purposes is further described below. The most appropriate therapy depends on the patient, the specific diagnosis, and the physician who is treating and monitoring the patient's condition.

From the foregoing discussion, it becomes evident that the disease that may be treated, according to the present invention, is limited only by the involvement of Sax2 protein. By involvement, it is not even a requirement that Sax2 protein be mutated or abnormal—the expression or overexpression of this gene may be sufficient to actually affect a therapeutic outcome.

a. Genetic Based Therapies.

One of the therapeutic embodiments contemplated by the present inventors is intervention, at the molecular level, to augment or disrupt Sax2 expression. Specifically, the present inventors intend to provide, to a given cell or tissue in patient or subject in need thereof, an expression construct to deliver a therapeutically effective composition to that cell in a functional form. The expression construct may be one which is capable of providing Sax2 protein to the cell; alternatively, and preferably the expression construct is one which delivers an siRNA, antisense or other nucleic acid-based construct for the disruption of Sax2 expression. It is specifically contemplated that the genes disclosed herein will be employed in human therapy, as could any of the gene sequence variants discussed above which would encode the same, or a biologically equivalent polypeptide. The lengthy discussion of expression vectors and the genetic elements employed therein is incorporated into this section by reference. Particularly preferred expression vectors are viral vectors such as adenovirus, adeno-associated virus, herpesvirus, vaccinia virus and retrovirus. Also preferred is liposomally-encapsulated expression vector.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for delivery. The section below on routes contains an extensive list of possible routes. For example, systemic delivery is contemplated. In those cases where the individual being treated has a tumor, a variety of direct, local and regional approaches may be taken. For example, the tumor may be directly injected with the expression vector. A tumor bed may be treated prior to, during or after resection. Following resection, one generally will deliver the vector by a catheter left in place following surgery. One may utilize the tumor vasculature to introduce the vector into the tumor by injecting a supporting vein or artery. A more distal blood supply route also may be utilized.

An “individual” as used herein, is a vertebrate, preferably a mammal, more preferably a human. Mammals include research, farm and sport animals, and pets.

b. Protein Therapy.

Another therapy approach is the provision, to a subject, of Sax2 protein polypeptide, active fragments, synthetic peptides, mimetics or other analogs thereof. The protein may be produced by recombinant expression means or, if small enough, generated by an automated peptide synthesizer. Formulations would be selected based on the route of administration and purpose including, but not limited to, liposomal formulations and classic pharmaceutical preparations.

In addition, the present invention details methods and compositions for identifying additional modulators of obesity such modulators may be used in the therapeutic embodiments of the present invention.

c. Combined Therapy.

In addition to therapies based solely on the delivery of inhibitors of Sax2 and related, compositions, combination therapy is specifically contemplated. In the context of the present invention, it is contemplated that Sax2 inhibition therapy could be used similarly in conjunction with other agents for treating obesity and obesity related disorders such as diabetes, heart disease, stroke, cancer, gallbladder disease and the like.

To achieve the appropriate therapeutic outcome using the methods and compositions of the present invention, one would generally administer a first therapeutic agent designed to inhibit Sax2 expression (or stimulate expression in those embodiments where increased Sax2 is required) as discussed herein and at least one other therapeutic agent (second therapeutic agent). These compositions would be provided in a combined amount effective to produce the desired therapeutic outcome. This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second therapeutic agent.

Alternatively, the first therapeutic agent may precede or follow the other agent treatment by intervals ranging from minutes to weeks. In embodiments where the second therapeutic agent and expression construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Local delivery of the first therapeutic agent (i.e., the inhibitor, stimulator or other, agent that decreases or increases the amount or activity of Sax2 in the individual) to patients may be a very efficient method for delivering a therapeutically effective gene to counteract a clinical disease. Similarly, the second therapeutic agent may be directed to a particular, affected region of the subject's body. Alternatively, systemic delivery of expression construct and/or the second therapeutic agent may be appropriate in certain circumstances, for example, where extensive metastasis has occurred.

### Use of Sax2-Based Compositions for Diagnostic Purposes

Preferred aspects of the present invention are directed to methods of diagnosing a disorder in which Sax2 is overexpressed or aberrantly expressed. In preferred embodiments, the diagnostic methods of the present invention are achieved through the detection of the Sax2 protein or a fragment thereof. Such a protein may be detected using antibodies specific for the protein in any of a number of formats commonly used by those of skill in the art for such detection.

For example, elsewhere in the present application, the production and characterization of monoclonal antibodies specific for Sax2 is described. Such antibodies may be employed in ELISA-based techniques and/Western blotting techniques to detect the presence of the full length Sax2 or a fragment thereof. Methods for setting up ELISA assays and preparing Western blots of a sample are well known to those of skill in the art. The biological sample can be any tissue or fluid in which Sax2 cells might be present.

An anti-Sax2 antibody or fragment thereof can be used to monitor expression of this protein in obese individuals. Typically, diagnostic assays entail detecting the formation of a complex resulting from the binding of an antibody or fragment thereof to Sax2. For diagnostic purposes, the antibodies or antigen-binding fragments can be labeled or unlabeled. The antibodies or fragments can be directly labeled. A variety of labels can be employed, including, but not limited to, radionuclides, fluorescers, enzymes, enzyme substrates, enzyme cofactors, enzyme inhibitors and ligands (e.g., biotin, haptens). Numerous appropriate immunoassays are known to the skilled artisan (see, for example, U.S. Pat. Nos. 3,817,827; 3,850,752; 3,901,654 and 4,098,876). When unlabeled, the antibodies or fragments can be detected using suitable means, as in agglutination assays, for example. Unlabeled antibodies or fragments can also be used in combination with another (i.e., one or more) suitable reagent which can be used to detect antibody, such as a labeled antibody (e.g., a second antibody) reactive with the first antibody (e.g., anti-idiotype antibodies or other antibodies that are specific for the unlabeled immunoglobulin) or other suitable reagent (e.g., labeled protein A).

In one embodiment, the antibodies or fragments of the present invention can be utilized in enzyme immunoassays, wherein the subject antibody or fragment, or second antibodies, are conjugated to an enzyme. When a biological sample comprising a Sax2 protein is combined with the subject antibodies, binding occurs between the antibodies and the Sax2 protein. In one embodiment, a biological sample containing cells expressing a mammalian Sax2 protein, or biological fluid containing secreted Sax2 is combined with the subject antibodies, and binding occurs between the antibodies and the Sax2 protein present in the biological sample comprising an epitope recognized by the antibody. These bound protein can be separate from unbound reagents and the presence of the antibody-enzyme conjugate specifically bound to the Sax2 protein can be determined, for example, by contacting the sample with a substrate of the enzyme which produces a color or other detectable change when acted on by the enzyme. In another embodiment, the subject antibodies can be unlabeled, and a second, labeled antibody can be added which recognizes the subject antibody.

Kits for use in detecting the presence of a mammalian Sax2 protein in a biological sample can also be prepared. Such kits will include an antibody or functional fragment thereof which binds to a mammalian Sax2 protein or portion of this protein, as well as one or more ancillary reagents suitable for detecting the presence of a complex between the antibody or fragment and Sax2 or portion thereof. The antibody compositions of the present invention can be provided in lyophilized form, either alone or in combination with additional antibodies specific for other epitopes. The antibodies, which can be labeled or unlabeled, can be included in the kits with adjunct ingredients (e.g., buffers, such as Tris, phosphate and carbonate, stabilizers, excipients, biocides and/or inert proteins, e.g., bovine serum albumin). For example, the antibodies can be provided as a lyophilized mixture with the adjunct ingredients, or the adjunct ingredients can be separately provided for combination by the user. Generally these adjunct-materials will be present in less than about 5% weight based on the amount of active antibody, and usually will be present in a total amount of at least about 0.001% weight based on antibody concentration. Where a second antibody capable of binding to the monoclonal antibody is employed, such antibody can be provided in the kit, for instance in a separate vial or container. The second antibody, if present, is typically labeled, and can be formulated in an analogous manner with the antibody formulations described above.

Similarly, the present invention also relates to a method of detecting and/or quantitating expression of a mammalian Sax2 by a cell, in which a composition comprising a cell or fraction thereof (e.g., a soluble fraction) is contacted with an antibody or functional fragment thereof which binds to a mammalian Sax2 under conditions appropriate for binding of the antibody or fragment thereto, and binding is monitored. Detection of the antibody, indicative of the formation of a complex between antibody and or a portion of the protein, indicates the presence of the protein.

The method can be used to detect expression of Sax2 from the cells of an individual (e.g., in a sample, such as a body fluid, such as blood, or other suitable sample). The level of expression of in a biological sample of that individual can also be determined, for instance, by flow cytometry, and the level of expression (e.g., staining intensity) can be correlated with disease susceptibility, progression or risk.

In other embodiments, the present invention also contemplates functional assays for determining the presence of Sax2 in a given biological sample.

In certain other diagnostic embodiments, the polynucleotide sequences encoding Sax2 protein may be used for the diagnosis of conditions or diseases with which the expression of Sax2 protein is associated. The biological sample can be any tissue or fluid in which Sax2-expressing cells might be present. Preferred embodiments include adipocyte tissue, neuronal cells, central nervous system cells, microglial cells, glial cells, and the like. Other embodiments include samples where the body fluid is blood, lymph fluid, ascites, serous fluid, pleural effusion, sputum, cerebrospinal fluid, lachrymal fluid, or urine.

In the amplification procedures, polynucleotide sequences encoding Sax2 protein may be used in hybridization or PCR assays of fluids or tissues from biopsies to detect Sax2 protein expression. Such methods may be qualitative or quantitative in nature and may include Southern or northern analysis, dot-blot or other membrane-based technologies; PCR-technologies; dip stick, pin, chip and ELISA technologies. All of these techniques are well known in the art and are the basis of many commercially available diagnostic kits.

In addition such assays may useful in evaluating the efficacy of a particular therapeutic treatment regime in animal studies, in clinical trials, or in monitoring the treatment of an individual patient. In order to provide a basis for the diagnosis of disease, a normal or standard profile for Sax2 protein expression needs to be established. This generally involves a combination of body fluids or cell extracts taken from normal subjects, either animal or human, with Sax2 protein, or a portion thereof, under conditions suitable for hybridization or amplification. Standard hybridization may be quantified by comparing the values obtained for normal subjects with a dilution series of Sax2 protein run in the same experiment where a known amount of purified Sax2 protein is used. Standard values obtained from normal samples may be compared with values obtained from samples from cachectic subjects affected by Sax2 protein expression. Deviation between standard and subject values establishes the presence of disease.

Once disease is established, a therapeutic agent is administered; and a treatment profile is generated. Such assays may be repeated on a regular basis to evaluate whether the values in the profile progress toward or return to the normal or standard pattern. Successive treatment profiles may be used to show the efficacy of treatment over a period of several days or several months.

PCR as described in U.S. Pat. Nos. 4,683,195 and 4,965,188 provides additional uses for oligonucleotides based upon the Sax2 protein sequence. Such oligomers are generally chemically synthesized, but they may be generated enzymatically or produced from a recombinant source as described herein above. Oligomers generally comprise two nucleotide sequences, one with sense orientation and one with antisense, employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Other nucleic acid amplification procedures include transcription-based amplification systems (TAS), including nucleic acid sequence based amplification (NASBA) and 3SR. Kwoh et al., 1989; Gingeras et al., PCT Application WO 88/10315; ligase chain reaction (“LCR”), disclosed in EPO No. 320 308, U.S. Pat. No. 4,883,750; Strand Displacement Amplification (SDA); Repair Chain Reaction (RCR) and the like.

Following amplification, it may be desirable to separate the amplification product from the template and the excess primer for the purpose of determining whether specific amplification occurred. In a preferred embodiment, amplification products are separated by agarose, agarose-acrylamide or polyacrylamide gel electrophoresis using standard methods. See Sambrook et al., 1989. In a preferred embodiment, the gel is a 2% agarose gel.

Alternatively, chromatographic techniques may be employed to effect separation. There are many kinds of chromatography which may be used in the present invention: adsorption, partition, ion-exchange and molecular sieve; and many specialized techniques for using them including column, paper, thin-layer and gas chromatography.

The amplification products must be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

However, it also is possible to determine the sequence of the amplification products without separation. These methods may be collectively termed Sequencing By Hybridization or SBH (Drmanac & Crkvenjakbv, U.S. Pat. No. 5,202,231). Development of certain of these methods has given rise to new solid support type sequencing tools known as sequencing chips. These techniques are described in numerous U.S. patents including e.g., U.S. Pat. No. 5,202,231; U.S. Pat. No. 6,401,267 and also WO 89/10977.

In certain embodiments, the amplification products are visualized indirectly. Following separation of amplification products, a nucleic acid probe is brought into contact with the amplified marker sequence. The probe preferably is conjugated to a chromophore but may be radiolabeled. In another embodiment, the probe is conjugated to a binding partner, such as an antibody or biotin, where the other member of the binding pair carries a detectable moiety.

In a particularly preferred embodiment, detection is by Southern blotting and hybridization with a labeled probe, according to standard protocol. See Sambrook et al., 1989. In such methods, the amplification products are separated by gel electrophoresis. The gel is then contacted with a membrane, such as nitrocellulose, permitting transfer of the nucleic acid and non-covalent binding. Subsequently, the membrane is incubated with a chromophore conjugated probe that is capable of hybridizing with a target amplification product. Detection is by exposure of the membrane to x-ray film or ion-emitting detection devices. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out the diagnostic (and prognostic) methods according to the preset invention.

Additionally, methods to quantitate the expression of a particular molecule include radiolabeling (Melby et al., J Immunol Methods 159: 235-44, 1993) or biotinylating (Duplaa et al., Anal Biochem 229-36, 1993) nucleotides, coamplification of a control nucleic acid, and standard curves onto which the experimental results are interpolated. Quantitation of multiple samples may be speeded up by running the assay in an ELISA format where the oligomer of interest is presented in various dilutions and a spectrophotometric or calorimetric response gives rapid quantitation. For example, the presence of Sax2 protein in extracts of biopsied tissues may indicate the onset of a particular disease. A definitive diagnosis of this type may allow health professionals to begin aggressive treatment and prevent further worsening of the condition.

### Screening for Modulators of Sax2 Protein

The present invention also contemplates the use of Sax2 protein and active fragments thereof in the screening of compounds that modulate (increase or decrease activity) of Sax2 protein. These assays may make use of a variety of different formats and may depend on the kind of “activity” for which the screen is being conducted. Contemplated functional “read-outs” include Sax2 protein binding to a substrate; Sax2 protein binding to a receptor, or any other functional assay normally employed to monitor fat deposition induced by Sax2 protein.

a. Assay Formats.

The present invention provides methods of screening for modulators of Sax2 protein activity by monitoring the endocrine effects of Sax2 in the presence and absence of the candidate substance and comparing such results. It is contemplated that this screening technique will prove useful in the general identification of a compound that will serve the purpose of altering the effects of Sax2. In certain embodiments, it will be desirable to identify inhibitors of Sax2 activity. In other embodiments, it will be desirable to identify stimulators of Sax2 activity.

As discussed herein throughout, Sax2 is likely an endocrine factor secreted from the mid/hindbrain boundary and the ventral midbrain as well as the ventral neural that exerts its effect on adipose tissue as well as other tissues. Sax2 is shown to increase fat deposition into adipocytes and its elimination has been shown to produce a lack of fat accumulation but produces otherwise differentiated adipocytes. As such, inhibitors of Sax2 activity identified herein will be useful in inhibiting, decreasing or otherwise abrogating the effects of Sax2 protein. Such compounds will be useful in the treatment of obesity and obesity-related disorders.

In alternative embodiments, stimulators of Sax2 will be identified that may be used for promoting, augmenting or increasing the therapeutic effects of Sax2 protein. Such compounds will be useful in the treatment of various disorders or conditions where it is desirable to increase fat deposition.

In the screening embodiments, the present invention is directed to a method for determining the ability of a candidate substance to alter the Sax2 protein expression or activity of cells that either naturally express Sax2 protein or have been engineered to express Sax2 protein as described herein. Alternatively, the present application teaches the production of Sax2 null mice. Such mice and cells therefrom will be particularly useful in screening for modulators of Sax2. For example, Sax2 may be supplied to the mice or cells derived therefrom (e.g., WAT or BAT therefrom) to determine the endocrine effect of Sax2. The cells or animals also may then be contacted with Sax2 in combination with a putative modulator of Sax2 function in order to determine whether fat deposition is increased or decreased as a result of the presence of the candidate substance.

An alteration in Sax2 protein activity, expression or processing in the presence of the candidate substance will indicate that the candidate substance is a modulator of the activity. Sax2 may be a transcription factor that affects the transcription of a variety of different genes. The effects of the candidate substance on such gene expression in the presence and absence of Sax2 may be determined and also may indicate that the substance is a modulator of Sax2 activity.

While the above method generally describes a Sax2 protein activity, it should be understood that candidate substance may be an agent that alters the production of Sax2 protein, thereby increasing or decreasing the amount of Sax2 protein present as opposed to the per unit activity of the Sax2 protein.

Inhibitors of Sax2 protein activity or production may identified in assays set up in much the same manner as those described above in assays for Sax2 protein stimulators. In these embodiments, the present invention is directed to a method for determining the ability of a candidate substance to have an inhibitory or even antagonistic effect on Sax2 protein activity. To identify a candidate substance as being capable of inhibiting Sax2 protein activity, one would measure or determine Sax2 protein activity in the absence of the added candidate substance. One would then add the candidate inhibitory substance to the cell and determine the Sax2 protein in the presence of the candidate inhibitory substance. A candidate substance which is inhibitory would decrease the Sax2 protein activity, relative to the Sax2 protein activity in its absence.

b. Candidate Substances.

As used herein the term “candidate substance” refers to any molecule that is capable of modulating Sax2 protein activity or expression. The candidate substance may be a protein or fragment thereof, a small molecule inhibitor, or even a nucleic acid molecule. It may prove to be the case that the most useful pharmacological compounds for identification through application of the screening assay will be compounds that are structurally related to other known modulators of obesity. The active compounds may include fragments or parts of naturally-occurring compounds or may be only found as active combinations of known compounds which are otherwise inactive. However, prior to testing of such compounds in humans or animal models, it will be necessary to test a variety of candidates to determine which have potential.

Accordingly, the active compounds may include fragments or parts of naturally-occurring compounds or may be found as active combinations of known compounds which are otherwise inactive. Accordingly, the present invention provides screening assays to identify agents which inhibit or otherwise treat the indicia of obesity. It is proposed that compounds isolated from natural sources, such as animals, bacteria, fungi, plant sources, including leaves and bark, and marine samples may be assayed as candidates for the presence of potentially useful pharmaceutical agents.

It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other inorganic or organic chemical compounds that may be designed through rational drug design starting from known agents that are used in the intervention of obesity.

The candidate screening assays are simple to set up and perform. Thus, in assaying for a candidate substance, after obtaining a cell expressing functional Sax2 protein, one will admix a candidate substance with the cell, under conditions which would allow measurable Sax2 protein activity to occur. In this fashion, one can measure the ability of the candidate substance to stimulate the activity of the cell in the absence of the candidate substance. Likewise, in assays for inhibitors after obtaining a cell expressing functional Sax2 protein, the candidate substance is admixed with the cell. In this fashion the ability of the candidate inhibitory substance to reduce, abolish, or otherwise diminish a biological effect mediated by Sax2 protein from said cell may be detected.

“Effective amounts” in certain circumstances are those amounts effective to reproducibly alter a given Sax2 protein mediated event e.g., fat deposition in WAT or BAT, from the cell in comparison to the normal levels of such an event. Compounds that achieve significant appropriate changes in such activity will be used.

Significant changes in Sax2 protein activity or function or fat deposition of at least about 30%-40%, and most preferably, by changes of at least about 50%, with higher values of course being possible. The active compounds of the present invention also may be used for the generation of antibodies which may then be used in analytical and preparatory techniques for detecting and quantifying further such inhibitors.

Proteins are often used in high throughput screening (HTS) assays known in the art, including melanophore assays to investigate receptor ligand interactions, yeast based assay systems and mammalian cell expression systems. For a review see Jayawickreme and Kost, Curr. Opin. Biotechnol. 8: 629 634 (1997). Automated and miniaturized HTS assays are also contemplated as described for example in Houston and Banks Curr. Opin. Biotechnol. 8: 734 740 (1997)

There are a number of different libraries used for the identification of small molecule modulators including chemical libraries, natural product libraries and combinatorial libraries comprised or random or designed peptides, oligonucleotides or organic molecules. Chemical libraries consist of structural analogs of known compounds or compounds that are identified as hits or leads via natural product screening or from screening against a potential therapeutic target. Natural product libraries are collections of products from microorganisms, animals, plants, insects or marine organisms which are used to create mixtures of screening by, e.g., fermentation and extractions of broths from soil, plant or marine organisms. Natural product libraries include polypeptides, non-ribosomal peptides and non-naturally occurring variants thereof. For a review see Science 282:63 68 (1998). Combinatorial libraries are composed of large numbers of peptides oligonucleotides or organic compounds as a mixture. They are relatively simple to prepare by traditional automated synthesis methods, PCR cloning or other synthetic methods. Of particular interest will be libraries that include peptide, protein, peptidomimetic, multiparallel synthetic collection, recombinatorial and polypeptide libraries. A review of combinatorial libraries and libraries created therefrom, see Myers Curr. Opin. Biotechnol. 8: 701 707 (1997). A candidate modulator identified by the use of various libraries described may then be optimized to modulate activity of Sax2 protein through, for example, rational drug design.

It will, of course, be understood that all the screening methods of the present invention are useful in themselves notwithstanding the fact that effective candidates may not be found. The invention provides methods for screening for such candidates, not solely methods of finding them.

c. In Vitro Assays.

In one particular embodiment, the invention encompasses various binding assays. These can include screening for inhibitors of Sax2 transcription factor activity, or for molecules capable of binding to Sax2 transcription factor, as a substitute of the receptor function and thereby altering the binding of the Sax2 protein to DNA. Binding assays could use DNA as the bait and by modifying the motifs of the Sax2 protein one could determine the factors binding to the specific motifs. In such assays, Sax2 protein or a fragment thereof may be either free in solution, fixed to a support, expressed in or on the surface of a cell. Either the polypeptide or the binding agent (i.e., the DNA to which the Sax2 binds) may be labeled, thereby permitting determination of binding.

Such assays are highly amenable to automation and high throughput. High throughput screening of compounds is described in WO 84/03564. Large numbers of small peptide test compounds are synthesized on a solid substrate, such as plastic pins or some other surface. The peptide test compounds are reacted with Sax2 protein and washed. Bound polypeptide is detected by various methods. Combinatorial methods for generating suitable peptide test compounds are specifically contemplated.

Of particular interest in this format will be the screening of a variety of different Sax2 protein mutants. These mutants, including deletion, truncation, insertion and substitution mutants, will help identify which domains are involved with the Sax2 transcription factor/DNA complex interaction. Once this region has been determined, it will be possible to identify which of these mutants, which have altered structure but retain some or all of the biological functions of Sax2.

Purified Sax2 protein or a binding agent can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link the Sax2 protein active region to a solid phase.

Other forms of in vitro assays include those in which functional readouts are taken. For example cells in which a wild-type or mutant Sax2 protein polypeptide is expressed can be treated with a candidate substance. In such assays, the substance would be formulated appropriately, given its biochemical nature, and contacted with the cell. Depending on the assay, culture may be required. The cell may then be examined by virtue of a number of different physiologic assays, as discussed above. Alternatively, molecular analysis may be performed in which the cells characteristics are examined. This may involve assays such as those for protein expression, enzyme function, substrate utilization, mRNA expression (including differential display of whole cell or polyA RNA) and others.

d. In Vivo Assays.

The present invention also encompasses the use of various animal models. Given the disclosure of the present invention, it will be possible to identify non-human counterparts of Sax2 protein. This will afford an excellent opportunity to examine the function of Sax2 protein in a whole animal system where it is normally expressed. The inventors have developed mice that lack Sax2 expression however, other animals may be developed with aberrant Sax2 protein functions (overexpression of Sax2 protein), one can provide models that will be highly predictive of disease in humans and other mammals, and helpful in identifying potential therapies. Such animals may serve as useful models of obesity and obesity related disorders.

Another form of in vivo model is an animal with a Sax2 protein mediated disorder, e.g., as described herein below, transgenic models may be generated using the teachings of the present invention. The animal model may be treated with Sax2 protein in combination with other agents to determine the effect on Sax2 protein function in vivo. Similarly, in tissues exhibiting overexpression of Sax2 protein, it is possible to treat with a candidate substance to determine whether the Sax2 protein activity can be down-regulated in a manner consistent with a therapy. The fact that Sax2 null mice are not obese, even after continued feeding, have low blood glucose and do not accumulate fat in adipocytes provides evidence that animals may be generated that will be useful models for testing various therapies.

Treatment of animals with test compounds will involve the administration of the compound, in an appropriate form, to the animal. Administration will be by any route that can be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood, cerebrospinal fluid (CSF) or lymph supply and intratumoral injection.

Determining the effectiveness of a compound in vivo may involve a variety of different criteria. Such criteria include, but are not limited to, survival, reduction of tumor burden or mass, inhibition or prevention of inflammatory response, increased activity level, improvement in immune effector function and improved food intake.

### Receptor Identification

Given the identification of Sax2 as an endocrine factor of the present invention, it will now be possible to identify the endogenous receptor for Sax2 protein and related agents. Once such a receptor is identified it may be employed in various therapeutic applications as well as in the identification of therapeutic compounds through screening assays similar to those described herein above for Sax2 protein.

A cDNA library is prepared, preferably from cells that respond to Sax2 protein. As the receptor may be located on one or more of neuronal cell, WAT-cells or BAT-cells, the cDNA library may be prepared from such cells. Radiolabeled Sax2 protein can also be used to identify cell types which express high levels of receptor for Sax2 protein. Pools of transfected clones in the cDNA library are screened for binding of radiolabeled Sax2 protein by autoradiography. Positive pools are successively subfractionated and rescreened until individual positive clones are obtained.

Alternatively, a degenerate PCR strategy may be used in which the sequences of the PCR primers are based on conserved regions of the sequences of known receptors. To increase the chance of isolating a Sax2 protein receptor, the template DNA used in the reaction may be cDNA derived from a cell type responsive to Sax2 protein.

### Transgenic Animals/Knockout Animals

In one embodiment of the invention, transgenic animals are produced which contain a functional transgene encoding wild-type or mutant Sax2 protein polypeptides. Transgenic animals expressing or over-expressing Sax2 protein encoding transgenes, recombinant cell lines derived from such animals and transgenic embryos may be useful in methods for screening for and identifying agents that induce or repress function of Sax2 protein. Transgenic animals of the present invention also can be used as models for studying indications of abnormal Sax2 protein expression.

In one embodiment of the invention, a Sax2 protein encoding transgene is introduced into a non-human host to produce a transgenic animal expressing a human Sax2 protein encoding gene. The transgenic animal is produced by the integration of the transgene into the genome in a manner that permits the expression of the transgene. Methods for producing transgenic animals are generally described by Wagner and Hoppe (U.S. Pat. No. 4,873,191; which is incorporated herein by reference), Brinster et al. Proc Natl Acad Sci U S A. 82(13):4438 42, 1985; Hammer et al, Nature. 20 26; 315(6021):680 3, 1985; Palmiter and Brinster, Cell, 41(2): 343 5, 1985 (which are incorporated herein by reference) and in “Manipulating the Mouse Embryo; A Laboratory Manual” 2nd edition (eds., Hogan, Beddington, Costantimi and Long, Cold Spring. Harbor Laboratory Press, 1994; which is incorporated herein by reference in its entirety).

It may be desirable to replace the endogenous Sax2 protein by homologous recombination between the transgene and the endogenous gene; or the endogenous gene may be eliminated by deletion as in the preparation of “knock-out” animals. Typically, a Sax2 protein encoding gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic mice are generated which overexpress Sax2 protein or express a mutant form of the polypeptide.

Alternatively, the absence of a Sax2 protein in “knock-out” mice permits the study of the effects that loss of Sax2 protein has on a cell in vivo. Knock-out mice also provide a model for the development of Sax2 protein-related abnormalities. The present invention teaches the production of such a knock-out animal. The mice produced herein were generated by replacing part of the Sax2 coding sequences with the lacZ gene. The Sax2 mutants exhibit a strong phenotype indicated by growth retardation starting immediately after birth and leading to premature death within the first 3 weeks postnatal. Intriguingly, the studies, also demonstrated a striking autoregulation of the Sax2 gene in both a positive and a negative feedback mechanism depending on the specific cell type expressing Sax2. The production of the mice and their phenotype is discussed in further detail in the Examples.

As noted above, transgenic animals and cell lines derived from such animals may find use in certain testing experiments. In this regard, transgenic animals and cell lines capable of expressing wild-type or mutant Sax2 protein may be exposed to test substances. These test substances can be screened for the ability to enhance wild-type Sax2 protein expression and/or function or impair the expression or function of mutant Sax2 protein.

a. Methods of Making Recombinant Cells and Transgenic Animals

As noted above, a particular embodiment of the present invention provides transgenic animals which express or overexpress Sax2 protein, or to replace the Sax2 protein with a different sequence (e.g., lacZ) to create a Sax2 knockout. Knockouts are exemplified herein below and these animals were leaner than their wild-type counterparts, lacked fat accumulation in differentiated WAT and BAT, and also had low blood glucose. Transgenic animals of the invention, recombinant cell lines derived from such animals and transgenic embryos may be useful in methods for screening for and identifying agents that repress the obesity-related activity.

In a general aspect, a transgenic animal is produced by the integration of a given transgene into the genome in a manner that permits the expression of the transgene. Methods for producing transgenic animals are generally described by Wagner and Hoppe (U.S. Pat. No. 4,873,191; which is incorporated herein by reference), Brinster et al. Brinster et al. Proc Natl Acad Sci U S A. 82(13):4438 42, 1985; which is incorporated herein by reference in its entirety) and in “Manipulating the Mouse Embryo; A Laboratory Manual” 2nd edition (eds. Hogan, Beddington, Costantimi and Long, Cold Spring Harbor Laboratory Press, 1994; which is incorporated herein by reference in its entirety).

Typically, a gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic mice are generated which express a gene of interest.

DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by is staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM Tris, pH 7.4, and 1 mM EDTA) and purified on an Elutip-D™ column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm is a UV spectrophotometer. For microinjection, DNA concentrations are adjusted to 3 μg/ml in 5 mM Tris, pH 7.4 and 0.1 mM EDTA.

Other methods for purification of DNA for microinjection are described in Hogan et al. Manipulating the Mouse Embryo. (Cold Spring Harbor Laboratory, Cold Spring Harbor, N.Y., 1986), in Palmiter et al. Nature 300:611 (1982); the Qiagenologist, Application Protocols, 3rd edition, published by Qiagen, Inc., Chatsworth, Calif.; and in Sambrook et al. Molecular Cloning: A Laboratory Manual (Cold Spring Harbor Laboratory, Cold Spring Harbor, N.Y., 1989).

In an exemplary microinjection procedure, female mice six weeks of age are induced to superovulate with a 5 IU injection (0.1 cc, ip) of pregnant mare serum gonadotropin (PMSG; Sigma) followed 48 hours later by a 5 IU injection (0.1 cc, ip) of human chorionic gonadotropin (hCG; Sigma). Females are placed with males immediately after hCG injection. Twenty-one hours after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% CO₂ 95% air until the time of injection. Embryos can be implanted at the two-cell stage.

Randomly cycling adult female mice are paired with vasectomized males. C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmaker's forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

b. Monitoring Transgene Expression

In order to determine whether the transgene has been successful incorporated into the genome of the transgenic animal, a variety of different assays may be performed. Transgenic animals can be identified by analyzing their DNA. For this purpose, when the transgenic animal is a rodent, tail samples (1 to 2 cm) can be removed from three week old animals. DNA from these or other samples can then be prepared and analyzed by Southern blot, PCR, or slot blot to detect transgenic founder (FO) animals and their progeny (F1 and F2).

The various F0, F1 and F2 animals that carry a transgene can be analyzed by any of a variety of techniques, including immunohistology, electron microscopy, and making determinations of total and regional area weights. Immunohistological analysis for the expression of a transgene by using an antibody of appropriate specificity can be performed using known methods. Morphometric analyses to determine regional weights, B and/or T cell counts, and cognitive-tests to determine dementia characteristics can be performed using known methods.

In immuno-based analyses, it may be necessary to rely on Sax2 protein-binding antibodies. A general review of antibody production techniques is provided elsewhere in the specification.

Transgene expression may be analysed by measuring mRNA levels in a given cell. Messenger RNA can be isolated by any method known in the art, including, but not limited to, the acid guanidinium thiocyanate-phenol: chloroform extraction method, from cell lines and tissues of transgenic animals to determine expression levels by Northern blots, RNAse and nuclease protection assays.

Additionally, transgene expression in a given cell also may be determined through a measurement of protein levels of the cell. Protein levels can be measured by any means known in the art, including, but not limited to, western blot analysis, ELISA and radioimmunoassay, using one or more antibodies specific for the protein encoded by the transgene.

For Western blot analysis, protein fractions can be isolated from tissue homogenates and cell lysates and subjected to Western blot analysis as described by, for example, Harlow et al., Antibodies: A Laboratory Manual, (Cold Spring Harbor, N.Y. 1988).

For example, the protein fractions can be denatured in Laemmli sample buffer and electrophoresed on SDS-Polyacrylamide gels. The proteins are then transferred to nitrocellulose filters by electroblotting. The filters are blocked, incubated with primary antibodies, and finally reacted with enzyme conjugated secondary antibodies. Subsequent incubation with the appropriate chromogenic substrate reveals the position of the transgene-encoded proteins.

ELISAs are preferably used in conjunction with the invention. For example, an ELISA assay may be performed where Sax2 protein from a sample is immobilized onto a selected surface, preferably a surface exhibiting a protein affinity such as the wells of a polystyrene microtiter plate. The plate is washed to remove incompletely adsorbed material and the plate is coated with a non-specific protein that is known to be antigenically neutral with regard to the test antibody, such as bovine serum albumin (BSA), casein or solutions of powdered milk. This allows for blocking of nonspecific adsorption sites on the immobilizing surface and thus reduces the background caused by nonspecific binding of antisera onto the surface.

Next, the protein-specific antibody is added to the plate in a manner conducive to immune complex (antigen/antibody) formation. Such conditions preferably include diluting the antisera/antibody with diluents such as BSA bovine gamma globulin (BGG) and phosphate buffered saline (PBS)/Tween®. These added agents also tend to assist in the reduction of nonspecific background. the plate is then allowed to incubate for from about 2 to about 4 hr, at temperatures preferably on the order of about 25° to about 27° C. Following incubation, the plate is washed so as to remove non-immunocomplexed material. A preferred washing procedure includes washing with a solution such as PBS/Tween®, or borate buffer.

Following formation of specific immunocomplexes between the sample and antibody, and subsequent washing, the occurrence and amount of immunocomplex formation may be determined by subjecting the plate to a second antibody probe, the second antibody having specificity for the first (usually the Fc portion of the first is the target). To provide a detecting means, the second antibody will preferably have an associated enzyme that will generate a color development upon incubating with an appropriate chromogenic substrate. Thus, for example, one will desire to contact and incubate the antibody-bound surface with a urease or peroxidase-conjugated anti-human IgG for a period of time and under conditions which factor the development of immunocomplex formation (e.g., incubation for 2 hr at room temperature in a PBS-containing solution such as PBS/Tween®.

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H2O2 in the case of peroxidase as the enzyme label. Quantitation is then achieved by measuring the degree of color generation, e.g., using a visible spectrum spectrophotometer. Variations on this assay, as well as completely different assays (radioimmunprecipitation, immunoaffinity chromatograph, Western blot) also are contemplated as part of the present invention.

Other immunoassays encompassed by the present invention include, but are not limited to those described in U.S. Pat. No. 4,367,110 (double monoclonal antibody sandwich assay) and U.S. Pat. No. 4,452,901 (Western blot). Other assays include immunoprecipitation of labeled ligands and immunocytochemistry, both in vitro and in vivo.

c. Methods of Using Recombinant Cells and Transgenic Animals

The transgenic animals of the present invention include those which have a substantially increased probability of spontaneously developing obesity (i.e., those that overexpress Sax2) and those that have a non-obese phenotype (i.e., the knock-out mice described herein), when compared with non-transgenic littermates. A “substantially increased” probability of spontaneously developing a particular phenotype means that, a statistically significant increase of measurable symptoms of that phenotype is observed when comparing the transgenic animal with non-transgenic littermates. For example, the tissues of the knock-out mice described herein were analysed and revealed differences in fat deposition as observed through histological studies and lowered blood glucose levels as compared to their littermates.

As used herein, such a “signal” indicates any stimulus, mechanical or chemical, which results in measurable symptom of a given phenotype (e.g., blood glucose level, fat deposits, weight; etc.). It is contemplated that the knockout mice and the transgenic mice that overexpress Sax2 may form one of a battery of screens for manifestations of obesity and related disorders in combination with for example, the ob/ob mice that are transgenic for leptin.

Coding regions for use in constructing the transgenic mice include the coding region for Sax2 protein. However, it is contemplated that transgenic mice also may be constructed using coding regions which encode a complete polypeptide, or a fragment thereof, as long as the desired function of the polypeptide is retained. The coding regions for use in constructing the transgenes of the present invention further include those containing mutations, including silent mutations, mutations resulting in a more active protein, mutations that result in a constitutively active protein, and mutations resulting in a protein with reduced activity.

The transgenic mice of the present invention have a variety of different uses. First, by creating an animal model in which the Sax2 protein is overexpressed and constantly activated, the present inventors have provided a living “vessel” in which the function of Sax2 protein may be father dissected. Additionally, the animals provide a vehicle for testing non-Sax2 protein related drugs that may ameliorate obesity. Thus, the transgenic mouse provides a novel model for the study of obesity and associated disorders. This model could be exploited by treating the animal with compounds that potentially inhibit the in vivo action of Sax2 protein and treat obesity and related disorders.

### Pharmaceutical Compositions

Where clinical applications are contemplated, it will be necessary to prepare the viral expression vectors, antibodies, peptides, nucleic acids and other compositions identified by the present invention as pharmaceutical compositions, i.e., in a form appropriate for in vivo applications. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention include classic pharmaceutical preparations. Administration of these compositions according to the present invention will be via any common route so long as the target tissue is available via that route. The pharmaceutical compositions may be introduced into the subject by any conventional method, e.g., by intravenous, intradermal, intramusclar, intramammary, intraperitoneal, intrathecal, intraocular, retrobulbar, intrapulmonary (e.g., term release); by oral, sublingual, nasal, anal, vaginal, or transdermal delivery, or by surgical implantation at a particular site, e.g., embedded under the splenic capsule, brain, or in the cornea. The treatment may consist of a single dose or a plurality of doses over a period of time.

The active compounds may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents; for example, sugars or sodium chloride. Prolonged, absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

The compositions of the present invention may be formulated in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

In the clinical setting an “effective amount” is an amount sufficient to effect beneficial or desired clinical results. An effective amount can be administered in one or more doses. In terms of treatment, an “effective amount” of polynucleotide, and/or polypeptide is an amount sufficient to palliate, ameliorate, stabilize, reverse, slow or delay the progression of obesity-associated disease states or otherwise reduce the pathological consequences of the disease. The effective amount is generally determined by the physician on a case-by-case basis and is within the skill of one in the art. Several factors are typically taken into account when determining, an appropriate dosage. These factors include age, sex and weight of the patient, the condition being treated, the severity of the condition and the form of the antibody being administered. For instance, in embodiments in which the antibody compositions of the present invention are being therapeutically administered, it is likely the concentration of a single chain antibody need not be as high as that of native antibodies in order to be therapeutically effective.

“Unit dose” is defined as a discrete amount of a therapeutic composition dispersed in a suitable carrier. Parenteral administration may be carried out with an initial bolus followed by continuous infusion to maintain therapeutic circulating levels of drug product. Those of ordinary skill in the art will readily optimize effective dosages and administration regimens as determined by good medical practice and the clinical condition of the individual patient.

The frequency of dosing will depend on the pharmacokinetic parameters of the agents and the routes of administration. The optimal pharmaceutical formulation will be determined by one of skill in the art depending on the route of administration and the desired dosage. See for example Remington's Pharmaceutical Sciences, 18th Ed. (1990, Mack Publ. Co, Easton Pa. 18042) pp 1435 1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Appropriate dosages may be ascertained through the use of established assays for determining blood levels in conjunction with relevant dose response data. The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies, are conducted, further information will emerge regarding appropriate dosage levels and duration of treatment for specific diseases and conditions.

In a preferred embodiment, the present invention is directed at treatment of human disorders that are caused by the presence or overexpression of Sax2 (as in the case of obesity), or may be alleviated by administering Sax2 (e.g., disorders that could benefit from increased fat deposition in adipose tissue). A variety of different routes of administration are contemplated. For example, a classic and typical therapy will involve direct, injection of a discrete area.

It will be appreciated that the pharmaceutical compositions and treatment methods of the invention may be useful in fields of human medicine and veterinary medicine. Thus the subject to be treated may be a mammal, preferably human or other animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkey ducks and geese.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Generation of a loss-of-function allele for Sax2 with lacZ. Linkage mapping and the partial genomic cloning of Sax2 was previously reported (Chen and Lufkin, Mammalian Genome 8, 697-708, 1997). Plasmid p1083 was generated by cloning an 18-kb NotI fragment into the SalI site of vector pTZ18R. An 11.5-kb XhoI-SalI fragment containing the Sax2 gene was subcloned into the XhoI-SalI sites of the Bluescript KS vector (pRS1). BspEI sites located in the predicted second exon and the second intron were used to create the targeting construct. The construct pRS1 was linearized with BspEI, and the oligonucleotide 5′-CCGGGTACGTA GGAATTCCATATGC (SEQ ID NO:5) was inserted to modify the BspEI sites and to add SnaBI and NdeI sites. A XhoI-SalI fragment containing the IRESlacZ/floxedneo cassette was treated with Klenow enzyme to create blunt ends. This fragment was inserted into the SnaBI site of pRS1, resulting in construct pRS14. The right orientation of the insert was determined by DNA sequencing.

ES cell culture and chimeric mouse production. To generate targeted gene disruption in embryonic stem (ES) cells, 10 μg of DNA of pRS14 was linearized by AseI and electroporated into 10⁷ cells as described previously (Wang et al., Mech. Dev. 99:123-137, 2000). Positive clones were selected by growing the ES cells in the presence of G418 at concentrations in the range of 150 to 450 μg/ml. Altogether, 192 G418-resistant clones were selected and analyzed by Southern blotting using a 5μ external probe (SpeI-XhoI), resulting in a 6.6-kb band for the wild-type clone and an 8.8-kb band for the mutant clone. Positive ES cell lines were microinjected into C5.7BL/6J blastocysts, and the resulting male chimeras were backcrossed to C57BL/J6 females for a mixed genetic background. For β-galactosidase staining, embryos were collected at stages E8.5 to E14.5 as previously described (Frasch et al., Development 121:957-974, 1995).

cDNA cloning. mRNA was isolated from embryos at stage E11.5 (Qiagen), and reverse transcription-PCR (Expanding Long Template PCR System; Roche) reactions were performed using specific oligonucleotides corresponding to sequences just downstream of the homeobox (5′AGGCGCTGACACCAGCGCGCCG SEQ ID NO:6) and 325 bp downstream of the predicted start codon (5′TCCTGGGGCGGAGCGGGCAGGGCGG; SEQ ID NO:7). Using these oligonucleotides, a partial cDNA was obtained and subcloned into the pT-Adv vector (AdvanTage cloning kit; Clontech).

RNA in situ hybridization. Sagittal, transverse, and coronal sections of wild-type and Sax2 null allele mutant embryos at stages E10.5 to E18.5 were prepared as previously described (Tribioli et al., Mech. Dev. 65:145-162, 1997; Wang et al., Mech. Dev. 99:123-137, 2000). Embryo tissues were fixed in 4% paraformaldehyde overnight, washed in phosphate-buffered saline, dehydrated through graded ethanol, given by two, changes of Americlear (Fisher), and embedded in Paraplast (Fisher) overnight under vacuum. Sections of 7 μm were cut and floated onto Plus+slides (Fisher), dried, and stored at 4° C. As a probe, construct p1083 was linearized with the restriction enzyme SfiI and antisense RNA was synthesized in the presence of 35S UTP using T7 RNA polymerase for in situ hybridization on sections of wild-type embryos. Autoradiography was performed by dipping the slides in a 1:3 ratio of H-2 O:Kodak NBT2 emulsion, air drying, and exposing for 3 to 7 days. Slides were developed in Kodak D19 and counterstained with hematoxylin. For RNA in situ hybridization experiments comparing wild-type and Sax2 null allele mutants, we used the partial cDNA (RS19) and construct RS37. Construct RS37 consists of a 2.8-kb NsiI fragment, containing sequences starting 1 kb upstream of the first exon to the second intron, subcloned into Bluescript KS vector. RS19 and RS37 were linearized with BamHI and EcoRI, respectively, and RNA was synthesized using T7 RNA polymerase.

### Example 2

### Initial Studies

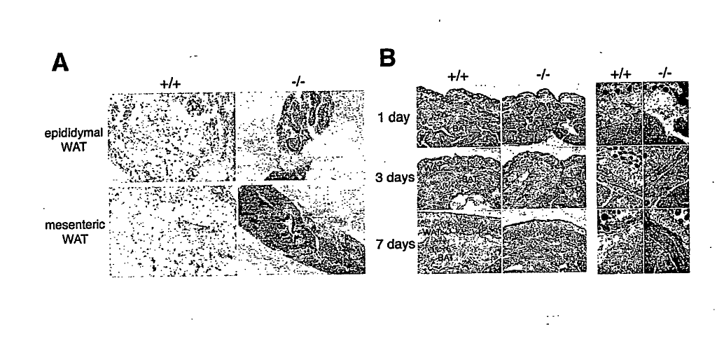

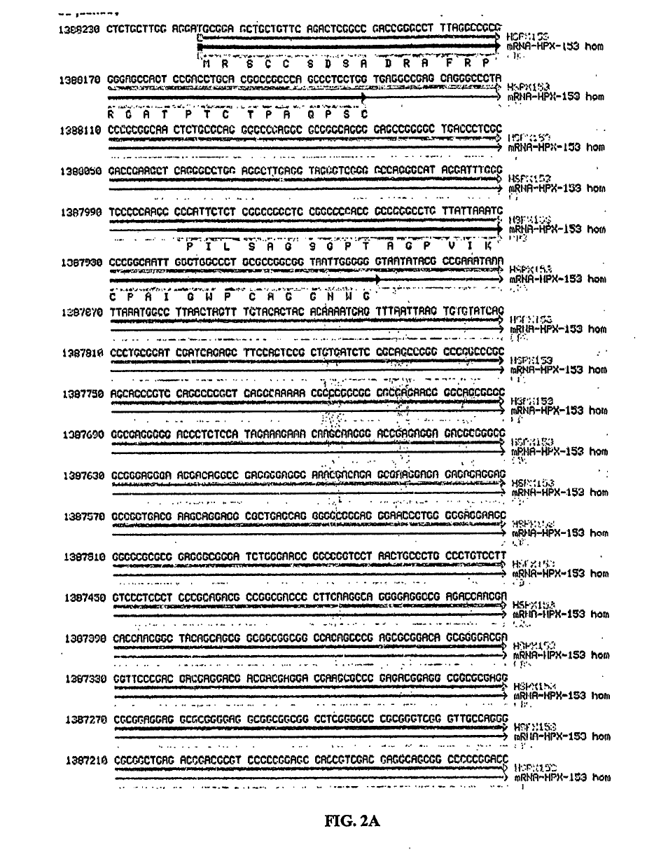

Histological analysis of wild-type and Sax2 null mutant tissues. Sax2 null mutants developed normally during embryogenesis and were indistinguishable from their littermates at birth but easily recognizable at day 3 postpartum due to their smaller size. To further determine the Sax2 phenotype, different tissues were examined by histological and biochemical methods. As a first step to determine the role Sax2 plays in energy homeostasis histological sections were obtained from paraffin embedded tissues of WAT and BAT at days 1, 3, 7 and 14 postpartum and stained with hematoxylin and eosin. In addition to adipocyte tissue, samples of heart and skeletal muscle, liver, pancreas and spleen also were examined. While muscle, liver, pancreas and spleen tissues do not show any difference between wild-type and the Sax2 null mutant, drastic differences were observed in both WAT and BAT as shown here for epididymal and mesenteric WAT at 2 weeks postpartum (FIG. 1A). Further sections through the neck portion showing a complex of WAT and BAT at day 1, 3 and 7 postpartum (FIG. 1B) revealing the lack of WAT and the reduction of BAT as early as day 1.

Analysis of glucose and TNFα levels in blood serum. Lack of adipocyte tissue strongly suggests an effect on the fat and/or glucose metabolism. As a first step to determine the cause of lack of adipocyte tissue glucose levels in the blood serum of wild-type and Sax2 null mutants were measured using a one touch glucose meter (Lifespan). While at day 1 and day 3 postpartum blood serum glucose levels remain the same for wild-type and Sax2 null mutants, at day 7 and 14 postpartum the glucose levels for the mutants reach only half the level of the wild-type. Lack of adipocyte tissue could also indicate cacchexia. Cacchexia can be easily determined by elevated levels of the cytokine TNFα and reduction of muscle tissue. ELISA assays performed on blood serum obtained from wild-type and Sax2 null mutants at day 7 and 14 postpartum did not show elevated TNFα levels in the mutant. These data combined with the absence of any obvious abnormality in the muscle tissue in the Sax2 null mutant, eliminate cacchexia as cause for the lack of adipocyte tissue.

Analysis of effect of high fat diet on adipocyte tissue in Sax2 null mutants. As mentioned above Sax2 null mutants show growth retardation starting at birth and weigh only half of their wild-type littermates (Simon and Lufkin, Mol. Cell. Biol. 23: 9046-9060, 2003). One explanation for the growth retardation and lack of adipocyte tissue might be the incapability of converting the food resources into fat storage. Wild-type mice fed high fat diet were found to gain a substantially increased amount of body weight and adipocyte tissue. To determine whether Sax2 phenotype can be rescued by high fat diet wild-type, heterozygous and homozygous adult mice were fed a 60% fat, 20% carbohydrate and 20% protein diet (Research Diets, N.J.) for 5 weeks and weighed daily. Data show a 27.5% increase of body, weight for wild-type and a 5% increase for Sax2 null mutant survivors while heterozygous animals exhibit an intermediate phenotype of 15% weight gain. These data suggest that Sax2 is required in diet-induced obesity.

RNA in situ hybridization on brain sections. RNA in situ hybridization determined Sax2 expression occurring early during embryogenesis and was primarily restricted to the nervous system, specifically to the mid/hindbrain boundary and the neural tube (Simon and Lufkin, Mol. Cell. Biol. 23: 9046-9060, 2003). Energy homeostasis in the brain is regulated by specific genes expressed in the hypothalamus and by serotonergic neurons. In a first approach to further determine Sax2 expression, and its interaction with possible target genes RNA in situ hybridization experiments were performed using marker genes involved in the energy homeostasis in specific nuclei of the hypothalamus, e.g. arcuate nucleus. First results indicate that the expression pattern of growth hormone and growth hormone releasing hormone are unchanged in the Sax2 null mutant suggesting that growth retardation of Sax2 null mutants is not due to a defect in the general growth regulation.

### Example 3

### Further Studies

Deleting the homeobox Sax2 gene resulted in a phenotype exhibiting growth retardation starting at birth and a high lethality rate within the first 3 weeks postpartum with an increased lethality rate between 2 to 3 weeks of age. Analysing the expression pattern of Sax2 during embryogenesis by beta-galactosidase staining and RNA in situ hybridization experiments revealed Sax2 expression being restricted mainly to the nervous system (Simon and Lufkin, Mol. Cell. Biol. 23: 9046-9060, 2003). Examining Sax2 null mutant pups within the first 2 weeks postpartum revealed lack of WAT and reduction of BAT starting at birth. Preliminary data determined decreased blood glucose levels of mutant pups starting at day 7 postpartum. In addition first results suggest that a high fat diet cannot rescue the Sax2 phenotype. The following example is directed at further studies designed to further describe the rote Sax2 plays in the glucose and fat metabolism.

Further histological analysis of tissues. Lack of adipocyte tissue can be caused by many events, defects in adipocyte differentiation and maturation, cacchexia and/or defects in glucose and fat metabolism. To further determine the cause of lack of adipocyte tissue in Sax2 null mutants different tissues will be analysed by histological as well as biochemical and physiological methods. To determine changes in cell structure resulting from the loss of Sax2 expression in the different tissues involved in or effected by the glucose and/or fat metabolism, e.g. epididymal and mesenteric WAT, BAT, brain, heart and skeletal muscle, liver and pancreas, will be collected from pups at different days postpartum and from adult animals of wild-type and Sax2 null mutants. Seven μm paraffin embedded sections will be prepared as described previously (Wang and Lufkin, Dev Biol., 227(2):432-49, 2000). These sections will be collected on glass slides, dewaxed and stained with either hematoxylin and eosin or oil red O stain essentially as described (Sheehan and Hrapchak, Theory and practice of histotechnology. Columbus: Batelle Press, 1987).

Embryos will be fixed in 4% paraformaldehyde overnight then dehydrated through graded ethanols, followed by Americlear and paraffin embedding. HE staining will be performed essentially as described (Lufkin et al., Proc. Nat'l Acad. Sci., USA, 90: 7225-7229, 1993). In addition to examining changes in cell structure sections will be stained for lipid content with oil red O in isopropanol as described (Sheehan and Hrapchak, Theory and practice of histotechnology. Columbus: Batelle Press, 1987). Paraffin embedded sections will be dehydrated through ethanol series, washed in distilled water, stained in oil red O working solution for 6 to 10 minutes, rinsed in water and counterstained in Harris' hematoxylin for 1 minute. Sections will be blued in 0.05% lithium carbonate and coverslipped as described (Sheehan and Hrapchak, Theory and practice of histotechnology. Columbus: Batelle Press, 1987). Paraffin sections are treated with organic solutions therefore only bound lipids will be visualized. To stain total lipid content oil red O staining will also be performed on frozen tissues embedded in OCT medium (Miles, Inc., IN) on dry ice and stored at −80° C. Sections of frozen tissues will be prepared using a cryostat, collected on glass slides, air dried for 20 minutes and stored at −80° C. Frozen sections will be treated similarly to paraffin sections for oil red O staining foregoing the dehydration steps. All stains will be obtained from Sigma.

Blood serum analysis for factors involved in energy homeostasis. Analysis of blood circulating factors involved in the regulation of the glucose and fat metabolism, e.g. insulin and leptin, will provide further insight into the function of Sax2 in these processes. As an example insulin is synthesized and secreted by the beta cells in the islets of Langerhans in the pancreas in close relation with the release of glugacon, released by the alpha cells. The relative amounts of insulin and glucagon released by the pancreas, are regulated so that the rate of hepatic glucose production is kept equal to the use of glucose by peripheral tissues. To further determine the pathways which are; altered by the deletion of the Sax2 gene, wild-type, heterozygous and homozygous pups at different days postpartum and adult animals will be anesthetized with avertin (0.015 g/g body weight) and blood samples will be collected by heart puncture and analysed for the levels of leptin, adiponectin, insulin, glycogen, glucagon, triglycerides, free fatty acids and β-hydroxybutyrate in addition to glucose.

Leptin and adiponectin levels will be determined by using ELISA based. Quantikine M kits by R&D systems. Glucagon levels will be determined by RIA kits from Linco, St. Charles, Mo. Serum insulin will be determined by ELISA obtained from Crystal Chemicals. Inc, Chicago, Ill. Triglycerides, cholesterol, β-hydroxybutyrate and free fatty acids will be determined by enzymatic kits obtained from Sigma and Roche Molecular Biochemicals. Because WAT is the major site of leptin and adiponectin synthesis, it is expected that their levels will be very low in Sax2 null mutants.

Preliminary data revealed a decrease in blood glucose level in the Sax2 null mutant starting at day 7 postnatal. It is possible that insulin levels are high preventing increase of glucose levels by accelerating glycogen synthesis in liver and muscle. On the other hand insulin levels could be low due to the low glucose level in the blood that could lead to a decrease in triglyceride formation and therefore lack of adipocyte tissue.

Determining the levels of triglycerides and free fatty acids in the blood will further determine at what point of the pathway Sax2 is interfering in the glucose/fat metabolism. A low insulin level would result in an increase of glucagon release that should stimulate gluconeogenesis and lead to elevated levels of glucose. Determining the level of glucagon will further narrow down the site of deregulation of the glucose/fat metabolism. Measuring β-hydroxybutyrate levels, one compound of the ketone bodies and an indicator for ketonemia, will provide clues as to the site of disruption in the glucose/fat metabolism. Ketohemia is most often seen in cases of starvation or severe diabetes mellitus. Ketone bodies are formed by liver mitochondria by diverting excess acetyl CoA derived from fatty acid or pyruvate oxidation into acetone or β-hydroxybutyrate. Ketone bodies are an important source of energy for peripheral tissues when glucose levels are low.

All assays described here will be performed as described by the manufacturers.

Analysis of tissue extracts for factors involved in energy homeostasis. In addition to the blood serum analysis for critical factors in the fat and glucose metabolism, RNA and protein extracts of different tissues, e.g. WAT, BAT, liver, pancreas, muscle and the brain, will be prepared and analysed by northern blot and western blot hybridization experiments, respectively. These experiments will also allow determining the level of molecules that are not circulating in the blood but are involved in the glucose/fat metabolism, e.g. leptin receptors, insulin receptors and glucose transporter in addition to factors described above.

Examining the different molecules at the transcriptional and translational level will further determine the interruption of the normal pathway. The homeobox Sax2 gene is a transcription factor expected to regulate target genes at the transcriptional level. It is possible that Sax2 activates or inhibits target genes, which in turn are regulating other factors involved in glucose/fat metabolism, and this regulation could be either at the transcriptional or translational level. The specific tissues under investigation will be homogenized and RNA will be isolated (Qiagen) for Northern blot hybridization experiments. Northern blot hybridizations will be performed according to standard procedures using probes for leptin, leptin receptor, adiponectin, glucagon, glucose transporter, insulin and insulin receptor. Protein extracts will be prepared by homogenizing tissues in SDS sample buffers and analysed by standard western blot procedures using antibodies for leptin, leptin receptor, adiponectin, insulin, insulin receptor, glucagon and glucose transporter. Probes for the Northern blot analysis are available as EST clones or will be prepared by RT-PCR using specific oligonucleotides for the different factors under investigation. Antibodies for the Western blot analysis will be obtained from Sigma (leptin, leptin receptor, insulin and glucagon) and Abcam (insulin receptor, adiponectin, glucose transporter).

Physiological analysis of Sax2 phenotype. To further determine the effect the lack of Sax2 gene expression has on the glucose/fat metabolism Sax2 null mutants and control animals will be exposed to a series of whole body tests.

High fat diet and fasting experiments. To further confirm that Sax2 is involved in diet induced obesity adult wild-type, heterozygous and mutant mice will be fed high fat diet (60% fat, 20% carbohydrate, 20% protein, Research Diets) for 5 weeks and weight daily. A pilot study on a small number of animals (n=3/group) showed no weight gain in Sax2 null mutant mice and an intermediate weight gain for heterozygous animals compared to wild-type. To conduct a more conclusive analysis further studies are required including a larger number of animals. An additional approach to determine a function for the Sax2 gene in energy homeostasis, wild-type, heterozygous and homozygous adult mice will fast for 48 hours and fasted and control animals will be sacrifice. Blood samples will be collected as well as adipocyte tissues (epididymal and mesenteric WAT, BAT), liver, muscle, heart, pancreas and brain from both the high fat diet group as well as the fasting group. The tissues will be either fixed in 4% paraformaldehyde for paraffin sections or frozen in OCT medium (Miles, Inc., IN) for cryostat sectioning as well as for RNA and protein extracts. Samples will be analysed as described in 1.1 to 1.3 in this section. In addition paraffin sections of the brain will also be employed for RNA in situ hybridizations as described above.

Determination of body temperature and temperature sensitivity. Total body energy expenditure represents the conversion of oxygen and food to carbon dioxide, water, heat and work on the environment. While WAT is the major tissue for energy storage and release of endocrines, BAT on the other hand plays a major role in thermogenesis or energy expenditure in form of heat (reviewed in Lowell and Spiegelman, Nature 404: 652-660, 2000). As mentioned above and shown in FIG. 1, BAT occurs normal at birth in Sax2 mutants but like WAT it fails to incorporate lipid droplets starting at birth followed by a reduction of the tissue. To determine whether the reduction of BAT is due to energy overexpenditure in form of heat, body temperature will be measured of adult wild-type and mutant mice as well as of pups at 1 and 2 weeks postnatal using a rectal probe attached to a digital thermometer (Comarks, Littlehampton, UK). To further determine the temperature and metabolic-rate Sax2 null mutant mice and control animals will undergo a cold-exposure experiment. The mice will be placed in a 4° C. room for 1 hour or overnight after a 12 hours fasting period and the body temperature will be determined. In addition blood samples and tissues will be prepared and analysed as described in 1.1 to 1.3 in this section. In particular BAT tissue will be analysed for cell number and lipid droplet content. Furthermore hormone-sensitive lipase activity will be determined by, measuring glycerol and free fatty acids released from explants of BAT and WAT that will be maintained in vitro.

RNA in situ hybridization analysis. Sax2 gene expression is restricted to the nervous system, the mid/hindbrain boundary, the ventral midbrain and the ventral neural tube, while the loss of Sax2 gene expression is affecting adipocyte, tissue suggesting an endocrine hormone function. To better understand the role Sax2 plays in the regulation of energy homeostasis it is necessary to determine its target genes. To determine which cell types and genes are affected by loss of Sax2 gene expression in situ hybridization will be performed on paraffin sections of the brain, especially on specific nuclei of the hypothalamus, e.g. arcuate nucleus, lateral hypothalamus, the ventral midbrain and on serotonergic neurons in the mid/hindbrain boundary, e.g. raphe nucleus, as well as on sections of WAT and BAT, with and without cold treatment, using the probes in Table. 2.

Coronal brain sections of wild-type and Sax2 mutant pups at day 7 and 14, as well as adult animals will be prepared as follows. The brain will be dissected and fixed in 4% paraformaldehyde overnight, washed in PBS and dehydrated through graded ethanol, followed by two changes of Americlear (Fisher) and embedded in Paraplast (Fisher) overnight under vacuum. Sections of 7 μm will be cut and floated onto Plus+slides (Fisher), dried, and stored at 4° C. 35S probes will be prepared using T3, T7 or Sp6 RNA polymerase. Autoradiography will be performed by dipping the slides in a 1:3 ration of H2O: Kodak NBT2 emulsion, air drying and exposing for 3-7 days. Slides will be developed in Kodak D19 followed by counter staining with hematoxylin. The PI has extensive experience with these methods. Problems could arise in the case that probes tested here do not change their expression pattern in Sax2 null mutants. To circumvent this problem additional probes will be employed that are involved in regulation of energy homeostasis and/or show a similar expression pattern as Sax2 in the tissues under investigation.

TABLE 2
Probes for RNA in situ hybridization

| Cell specificity | Probe |
|---|---|
| Hypothalamus | Neuropeptide Y, Agouti related peptide, |
| | Leptin, Leptin receptor, Pro- |
| | opimelanocortin, Insulin, Cocaine- and |
| | amphetamine-regulated transcript, |
| | galanin |
| Ventral midbrain | Tyrosine hydroxylase, NurrI, Ptx3, 5- |
| | HTR2C, Serotonin |
| Serotonergic neurons | Tryptophan hydroxylase, Pet1, Leptin, |
| | Leptin receptor |
| WAT | Leptin, Adiponectin, C/EBPα, PPARγ |
| BAT | UCP-1 |

Screening for potential Sax2 target genes. Even if the probes used for the RNA in situ hybridization experiments shown in Table 2 have all been shown to be involved in the regulation of energy homeostasis, it is possible that none of these genes will show a different expression pattern in the Sax2 null mutant. This can be explained by the possibility that Sax2 acts downstream of the tested genes or Sax2 target genes are not yet known. To address this potential problems microarray as well as real time RT-PCR assays will be employed.

Microarray assays. Microarray assays-provide a powerful tool to screen a large number of genes to determine target genes for Sax2. Total RNA will be prepared from different tissues, especially the brain, WAT, BAT, muscle and liver, of wild-type and Sax2 null mutants from adult as well as 2 week old pups. To enhance differences in the expression pattern of possible candidate genes, RNA will be isolated from adult animals, wild-type and Sax2 null mutants, after being exposed to high fat diet. Sax2 expression starts early in embryogenesis and it is most likely that the phenotype is already established during embryogenesis, therefore RNA will also be prepared from embryos at stage 13.5 as well as 1 and 2 week old pups and used for microarray assays. The RNAs will be reverse transcript to DNA in the presence of Cy3- or Cy5-dUTP and hybridized as described (Wurmbach et al, Methods 31: 306-316, 2003). Clones that show a different expression pattern in the mutant versus the wild-type will be further analysed by Northern blot hybridization, DNA sequencing and real time RT-PCR

Real time RT-PCR. As mentioned above, microarray assays have the advantage of screening a large number of transcripts simultaneously. However microarrays have several limitations, they require more RNA than real time RT-PCR and they do not provide an accurate quantitative analysis of gene expression. To address these problems and to further define genes identified by microarray assays, real time RT-PCR with RNAs prepared from different tissues, especially adipocyte and brain tissues, will be performed. Furthermore genes that are known to have a function in adipocyte differentiation and energy homeostasis, e.g. C/EBPα, PPARγ, ADD-1/SREBP-1, NPY, POMC, will be analysed for their expression pattern in wild-type and Sax2 null mutants. Analysing the expression pattern of these genes will be helpful to understand their relationship with the Sax2 gene product and how deletion of the Sax2 gene causes lipoatrophy and related diseases.

Certain experiments have shown that there is an increased level of serotonin in the hindbrain of Sax2 null mutants at day 1 postnatal. These data suggest that Sax2 regulates the energy homeostasis by controlling the expression of factors involved in the serotonin pathway.

The above experiments are designed to further elucidate the downstream effects of Sax2, however, as can be seen from Figures and the discussion provided herein, Sax2 is involved in the regulation of obesity and inhibition of this gene expression and/or protein activity will be useful in the treatment of a variety of obesity-related disorders.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference.

1. An isolated recombinant nucleic acid encoding a SAX2 polypeptide wherein said polypeptide is expressed in brain tissue, the polypeptide being encoded by the nucleic acid sequence presented in SEQ ID NO: 1, SEQ ID NO:3, SEQ ID NO:4, or SEQ ID NO:8.

2. An isolated recombinant nucleic acid encoding a recombinant protein having the amino acid sequence of SEQ ID NO:2 or SEQ ID NO:10.

3-4. (canceled)

5. The isolated nucleic acid of claim 2, wherein said nucleic acid is genomic DNA or cDNA.

6. (canceled)

7. An isolated polynucleotide that encodes a SAX2 protein and hybridizes under high stringency conditions to a nucleic acid of claim 2 but does not hybridize to a sequence that encodes SAX1.

8. A polynucleotide 8 to 80 nucleotides in length targeted to a nucleic acid molecule encoding SAX2, wherein said polynucleotide specifically hybridizes with a nucleic acid molecule of SEQ ID NO:1 and inhibits the expression of SAX2.

9. The compound of claim 8 comprising 12 to 50, 15 to 30 or 20 to 25 nucleotides in length.

10-11. (canceled)

12. The compound of claim 8 wherein said compound is a DNA or RNA antisense oligonucleotide.

13-14. (canceled)

15. The compound of claim 8 wherein at least a portion of said compound hybridizes with RNA to form an oligonucleotide-RNA duplex.

16. The compound of claim 8 having at least about 70% 80%, 90% or 95% complementarity with a nucleic acid molecule of SEQ ID NO 1.

17-19. (canceled)

20. The compound of claim 8 having at least one modified internucleoside linkage, sugar moiety, or nucleotide.

21. An expression construct comprising an isolated recombinant nucleic acid of claim 2 and a promoter operably linked to said polynucleotide.

22. The expression construct of claim 21, wherein said nucleic acid comprises a mature protein coding sequence as set forth in SEQ ID NO:1.

23. The expression construct of claim 21, wherein said expression construct is an expression construct selected from the group consisting of an adenoassociated viral construct, an adenoviral construct, a herpes viral expression construct, a vaccinia viral expression construct, a retroviral expression construct, a lentiviral expression construct and a naked DNA expression construct.

24. (canceled)

25. The recombinant host cell of claim 26, wherein said nucleic acid comprises a mature protein encoding sequence as set forth in SEQ ID NO:1.

26. A recombinant host cell stably transformed or transfected with an expression construct of claim 21, in a manner allowing the expression in said host cell of a protein of SEQ ID NO:2 or SEQ ID NO:10.

27. The recombinant host cell of claim 26, wherein said host cell is a mammalian cell, a bacterial cell, a yeast cell, or an insect cell.

28. (canceled)

29. An isolated and purified protein comprising an amino acid sequence that is 90% identical to the sequence set forth in SEQ ID NO:2 or SEQ ID NO:10.

30. An isolated and purified peptide comprising about 10 to about 50 contiguous amino acids of SEQ ID NO:2 or SEQ ID NO:10.

31. (canceled)

32. A purified antibody that is specifically immunoreactive with the protein of claim 29.

33. The antibody of claim 32, wherein said antibody is a monoclonal antibody.

34. A hybridoma cell line producing a monoclonal antibody of claim 33.

35-39. (canceled)

40. A method of inhibiting the expression of SAX2 in cells or tissues comprising contacting said cells or tissues with the compound of claim 9 so that expression of SAX2 is inhibited.

41. A method of decreasing fat deposition in a mammal comprising inhibiting the expression or activity of SAX2 in said mammal.

42. The method of claim 41, wherein said decrease in fat deposition manifests as a decrease in the white adipocyte tissue (WAT) of said mammal, a decrease in the brown adipocyte tissue (BAT) of said mammal, or a decrease in both WAT and BAT upon inhibition of expression or activity of SAX2 in said mammal.

43-49. (canceled)

50. The method of claim 41, comprising administering to said mammal a therapeutically or prophylactically effective amount of the compound of claim 9 so that expression of SAX2 is inhibited.

51. The method of claim 41, wherein said mammal is a human.

52-54. (canceled)


SEQUENCE LISTING (12 sequences)

SEQ ID NO: 1 - DNA, 5774 bp, Mus musculus
  misc_feature (4)..(4): n = a, c, g, or t
gatnaananntaantttgaattccnaatataaaaagtgtannaannnnganccaaaancncctntntaatanntnaattntcctantttttccananttttatnnaggcanagngagnnagggtnataaaaaatcnantgagntnngntataantatanatttttaacnaannttgttnccaaanntggaaaaaaaatncnagtnaanggcaacgaaaacccnaaantctggaananngcccccnnaangggactaaaaaccnttgaaaaaattnanaaccccnaaanatcncaantttcncttttctgtntaanaanngnaagncgcaaaaaacccntggtncccaggtatagccccanaaccccngtgtnncacgnaagcangaaaaacnntgcatanccaggtacccttangttggaatttcaagagccaacttcnggggngcgtgggttcnccangtcnttgggncntntgnagccctccacaagcaaattgatcccgcaatnatttaaaaagnggactgtctcnttccccnttcgccaagaagacggggcacgcaagtttccagccccgngaaggcgcagcggcacggcgctgagcaccccagcctccagtggactgtgctctggctccgggcagggcgtctgtctgtccgtctcatgcggctcagtccgtgagtcccgtaccgggcccgcctactccgcttgggaccgttcgcagccaattaggcgtgagcgctaacgagggaagcggaaccctgcggccgcctgcgggcgcacgtgtgtgcggcgtgtgggcgcgtgggcgcctccggcggggccgccataaatgcactggcccggcgcggcgggcgcctgaagcggtggcggaggcaaggccaggcagcaccagcagagcgggaacagctgggctgaggctgattttgcggggcgcagggatgggatcccagtaggtagcgggggcccgagccgcctgagcggcccgaggccggcatgagtaccagcgggcccggcggcacccggggacgtccccgcgctgccaccgccaccgcccgggcccggctcggggcccgcaccgccggctcctgcggctacagctcgggacactatggacggacgagctgagctgcccatctttccccgggctggagtgccaccgctcgcggccagcgacactgttcccgcggtgccagagggggctggagcggcccggcccgccgcgcccccgcgccccacctccttctccgtgttggacatcctagatcccaacaagttcaacagtaggagacgccgctgcgtgctactgggccctgtggtgcccgctacgtgtgccccatgcgccccggccgcgtgcgtcgcggtccccgctgcctccggacgttcgccgcgcgcagagctagaacgccgagccctctccgccgccacaggagtcgcagcggccgcgggagctgagcctacaagtgagtataaggggttggtgataggggcgaggacaccgactgctccggaatgtttatgctcccggcgagctgggcctgacccaagcgccgcggggctctgtggaaagttcgcagagcgcgggtccgccccaagcatccgtggctctcccgacccggattagggccctcactgctgcacgtttctggaaagttctgttgggccaaatctggggccttggggttgcgaactacggtccaggattcatctagtcaatctttggtgttggagggtagttggtgctggaggcaaagggacatgcggcgactgacttggtgatttacccacccccaccgtggccttcccggccctccccctttggacaaatcagactaattgtgacaaaacgctggttatctctggaaaaacaattaaatcccttcgattacagttaaagggaaatgtgcttagcggcggaaagcggccggtaatggggcggcccgcgccccctttgcggtcgatatcccattacggcgcatggcatatctctttcaagcaccttgcaaactccccccgaacatttaaattatagggtcaaaattcggataattactcgattaaaacctattacacttattaggggccgctattgatcgcgaattgcattcgaccagctcctgattgctttttgtgcccctcccccgtcctcctctaacttcttggcccctgcgcctctcatggaccagaacgttnagggagaantggaatggggccgccagctttaccaangcggttgcattgaccttccgttcctagagagaggaagggcaggccatgcaggctgcagttggacccctctctccgccaaggggttggtctacgaggaactcgcggctgcctgctggagctgtgtagggagggagcgagcgctgcacagttgggagagggcagccaggcagggctggggcagggatgactggctacgtgtcgcctgagggaatagttgtcctgctaagacctgggagttgaggacaggacacccgcactcttcatgcgacctgggtcatttgagctgggttattttcgagttgcgattttatgctccagtatgtcagcgtcaactcgatctgagagctgaacaccggccctgggtcggaaaacgtggaggcctcctctggcatttaaagaccacctgtgggtgaggggcatgaaaggtcctcagattcctgcagagcagacagacagccttgcaaaatttcagaggcaaaatgcagtcatggcggcagcatctctctggatgcagcgggattagcgctcatactccctcccagggccctgcttagaggttcacaatacgctttcccagcctccaaactcaaccagcccacgggaagctggagcccgcttaagacagagtcgctggggagctggttggtgggccccagaggtcccgcagggaagtcagcactttgcgacgcctagttttcagggcctgaagcctgaggtagggacagctcccagacgaccgattttggttttcagaagtggcctcctcacagccggccgaccccaccccaggcctcgcgcccctccagctattaactgtgcggtaattggctggccccgcgcccgctgtaattggaaggtaatatacgcccaaataaattaaatggactttaactagtttgtacattacacaaaatgagtttaattaagtttgtatctgccccgtgaatcgatcggagctctttttccgctgtacaatccgagggccgcccgcgcaccgcagccctaaaggccaaggccaggactgcggatggggcagcaaagcagctcgacacggagtgggtccttccgaaggaaggcagagaggaaatgcaagcaatcaaggaggaactgtggcaaggaggaagaaaaagaaaatctttggtgagggggcaaacgaggaagaggagaaacacaacagttaaaagagaatctctcctggggcggagcgggcagggcggctggcagcgggcctgaccctctgttctcttccttttcgccgcccgcaggtgctggggactcttacagggcggacgaggccgaggccaacggctacagcagcggcagcggtcgcagcccgaccgcggacagcgaggatgaagcgccggaggacgaggacgaggaagaagcgcccgaggtgcaggatgctcagggcacggaggagcctcggggaggcagcggcggtctcggggcccgcgggtcgggctgccccggagcggccgaggtcgaggcttcccctgtggacgatactgcggccccagggccccgtgggaactcgcccggagccccgggcccgcctgcaactgctacgggagcggggagcgcggggagtaccccgcagggcgccgcccgtgaccaccaagcccaagcggaagcgcacgggctcggactcgaagtccgggaagccgaggcgtgcgcgcaccgccttcacctacgagcagctcgtggcgctggagaacaagtttaaggccactcgctacctgtcggtgtgcgagcgcctcaacctggcgctgtcgctgagcctcaccgagacgcaggtgaagatctggttccagaaccgccgaaccaagtggaagaagcaaaacccaggcgctgacaccagcgcgccgaccggcggcggcgggggccccggcccgggagcggggcccggcgcggggctgcccggcggcctcagtccgctcagcccctcgcctcccatgggcgctccgctcgccttgcacggcccggccgggtacccggcgcacagcccaggagggctggtgtgtgtgcgcagctgcccttcctgtccagcccggccgtgctgtcgcccttcgtgctgggctcgcagacgtacggcgcgcccgccttttatgcgccccacctctgaatctcggtgccaggaccccggtggggaccgcagcacgcggcctcaaggccagaggacccaaggcgagcatgggcgtctcttttctaaggtttcagtcgttggtcctgtatatagatagccgttcctacagaagagaaatttataaacgagtgtgcgcaggggccaaacgggaggctcccgtggcctgctccaagcactttgtagaactcttaataaagcacaggaagccatgcgggcgttttcccgaaggctctggcgccgtaggatgccgatcccgccttcctgtccgctccggttcttatccatctgtgtgtcgccctgggcagggacttcagagcggttaggtatagtttcctgtcagtgtttagtgtctcatggtcactctagtagcttgcactctgtcacctttctctcttggggatcgccctgggtccagcacgggtctgagtccccttcccgctctctccgaagtgaggccctttcccctcttcttgcagcctggcactgctgacctccaaatccggtcccaccaactgcccccactcccacccccaagcaggctacttgtgggcaggaaaaacatcccccttcctagaggaggaagctggagggttgaggtgtaacaacctgccagatagtttattagccagaatggaagtgtgtgtgtgtgtgtgtgtgtgtgtgtgtgtgtgtgtgtgtagtccctatgtgctgaacgcatggagcccaggagtggcccgaagctgtggcccacacaccccaaagcagataccgaaccacctatgactacagggagactacatggcagcctgttccagctttggaagattttgtatatcagtaggggtggagactggagaaggaaaagggggttgactgtggagagggacagggaggcaactgttttttggtctgttccagggtccctgantttccctagaaagaacaaaagagcctgctgtgttccactgtggggctaggcttntccttcaggcgtcctgtttcctagcacagtgagcttttggaggggtcttgaccgctagtcaagcgagctgcctttgggagtcaggctgagagtgaactgcagtcactagcaatggtcagagtgcacaaaactggcttcatgctcatcaaaagccaaaggcaggtaagagcagcatctggcagtgagggggcaggaagccaacctgccccttgaagactggtagcttccctcactgaccaggagagacctggtatcagattccagccagcccacattccactttcagaccttctaggcctaaaggttcctgtcctgacctgatgtttgacagccctagatgggcacctggtccagccttctagttcagtggtgcaatactactatnccctgtggtcagggagtgagagccatgtatgaagtggcttaactgtgagaggtgat

SEQ ID NO: 2 - protein, 527 aa, Mus musculus
MDQRGSLPAVIAKDRPCAEQALGTVVGGGMHTRWSTRRRRTHSHSGHASSIAGSHLGPSEAGQLIGLSERRRKTRKRSIGYLTLILQSRARPSEVVRRDCPVNLQTGQDELERRALSAATGVAAAAGAEPTRPCLEVHNTLSQPPNSTSPREAGARLRQSRWGAGCPKGQGQDCGWGSKAARHGVGPSEGRQRGNASNQGGTVARRKKKKIFGAGDSYRADEAEANGYSSGSGRSPTADSEDEAPEDEDEEEAPEVQDAQGTEEPRGGSGGLGARGSGCPGAAEVEASPVDDTAAPGPRGNSPGAPGPPATATGAGSAGSTPQGAAVTTKPKRKRTGSDSKSGKPRRARTAFTYEQLVALENKFKATRYLSVCERLNLALSLSLTETQPGRAVALRAGLADVRRARLLCAPPLNLGARTPVGTAARGLKARGPKASMGVSFLRFQSLVLYIDSRSYRREIYKRVCAGAKREAPVACSKHFVELLIKHRKPCGRFPEGSGAVGCRSRLPVRSGSYPSVCRPGQGLQSG

SEQ ID NO: 3 - DNA, 17442 bp, Homo sapiens
ggctagacagatggaccaggatgtcagacaactaaaagggtggaggtggggtgcacaggctgaaacactgccccacctgccagcattgggcccttcactcttccctgtcccagaatgcttttgccttttgcatcctgcctctacagtctcgggggcacgggggcgggggggggatccgccagtgaacagataaaggtcacgtgacgtgttaaggcagaactgtgaaagcttgggttggaggggtgcacaggatggcacctgccttacttgaggaaatcagggtggcctccaggtggtgctgccacgaaggggagcagggatgatgcatgtgcactagtgagtggctaagggcaccgagaggacatggcagctcggccatccaggcacagggaggggctcggagtaaccaccagcagagaggacatggcagctcagccatccaggcacagggaggggctcggagtaaccaccagcagagaggacatggcggctcggccatccaggcacagggaggggctcagagtaaccaccagcagagaggacagctcggccatccaggcacagggaggggcttggagtaccgggtacaggcctagggagcatacaggatagctggaaggatccagccaggaatggcccctgataccaggctgcagagcctagacatggtccctggcacttctggaaggggaggagtactattcaatttacattttagaatgatggaaagcagacaggaggcagccagtctggggcagtggcaggcagaggccaggcggccaaggcacccccagacccccactcaggaggggtctcctgaccatgtgtctccaggcaacatgactctggtgattcccacacagacccctagacgactgtggcagccacagacaggagaatgcctatgcttcaagccctgagtaccagcaggagcttggggacaagggagggaatgtggccatcccaggcaggctccaacctgggcatgctgctgccccatgcagtgtagccagtggcaggcagaggccagagagggcaggggcaataagagagagtctggagggacagagcctgaggagtaaggacccctctgggcttggatgctttctctcagagaccagtcacacaattacctatgactttgcacatgaaaaacgctgtgtgaaaggaacccagcgttcacataaaaatcctaccacttaccggccaggggcactgagcacagcgtgggtcctcactggcctcaggctatgcatctgtaaggtggtaatccctgccccggcctgaccctgtggtcggtaaaccctcaggagccagggtgctctccaagttcaaccagcagcacccgggagagggagccagccgcggccgcctctgcagccctgatgggaagccaggcgtcctgccctccacatagcacccatgtccccagttgttccaattgtttgtatcttcttgtttttaagtttaaagggtttcagcctcacctttgcatctctggtgggctcctcgccagaagccaggccctgtgccacgtgctttccctccttaactcgcgcagcttgcaggtaggagaatggaagcccacgcggtgagcggcctggtaaacttctggccctttggtggatgctggcttggtcgaggtgaacgggctggcttttgatcgggctgtgtaatgtacgccgtgccaagactccacacacagcctgccgccctcttcctcccctggggcatcccaaagtttgagaaggagtcgagggcggggcagggaggtcagagacggcgggctggaggccaggagaccctcggagttgctggcgctgtccgcggtgctgaacacggcccgagcccgtggcctccgggggcgaggtgggagcgcgctgtccgcggtgctggcgctgttcgcagtgctgaagctgccctgtgcggaggcacacgaggctggggccgcgggtctccagagctgacgtgctctcgaggtcagagcctgtgtcccctctcgcttctggtgtcccgagtgtggtacactcattctggaggagtcacaggcccctaagtgggcatatcttagagaaggaacccaggcctgaaagcagagggctcccctctgacttcccaaactccggtgatcaacatgggtcgccgtggcgcgagtgtgtgcaggggcgagaagaggggacacaggagcctccttccccgtcctccgcccgcaccccccgcccccgtccggccgatgcaaggaggtgcacagcgaggctgtggggccaagtgtggggtggagtgcacttcgcacgaggcaaacactatacaaagtggaggtgattatgcctctgcccaaccctagagcccctgggaccctgctgcctttccagcctatccagctaaagctttccaggcaaagagagaagggacttccctccatgcaccaagtaaaaggctgtgaccttgtcgtcatcattcaccccatgaggggagggtatcaggacctggaagctcgcatgggactcaacccctacaattgacagatgaaaaaaatgcggcggggggtggttcataaacccatgctgagttcgtggcagtggtggctggctccccctcttcctcttgtccctcaaactttaaaaacctctaatgctggggagaaaatcctcgaagtctcagctcccacccactcctttcctcatgctcccttctaagaccctttaccaccacccatacttctgctcctgtcccaggcgtgttcctgccaacgcccacacccaagcccatcgtgaccccacctggacctgcagcaggggagagctctgcaatggagggaatgtttcctgtgcactgggcaggcaggggcagcccacccccggcactgatttcacctgtcagtgcccacctggggccctgcaagtgaagctggtgtcttggagataacccgagatggcagatgggcagagattgcacaaatgggggtctaattgacaatcaattgtgattaggagtgaaaggacgtctttatagccccagacatgctaggttgagatcggggaggggaggtatgtttctggggatctgagagaggaggaggggtgccctgcatctctgtggcttaccttgttcctcagagatgggggaggggtggggcaggggagcatgcagcgatgccgtctccaagcctttcccaggggaggcgacgggaggtgccctgtgtagctccgggggtcccctcaggctccaccacccggcccctgctcctctgaacctgactgagaacttcgaagttcattaccctgcccgctgcggctcacacgaggatggtgtggggcagagaggggaggggagggaaggctcgaggagaaccttggggcccacaaacttcagatagccacgtgctttcgtcaaagtggcactcaaccctggccagcccccgcccccatcagggagaacattctgcagtgctcgatgtgcacgtgtttgctgggaaacgagtcataattgtatattataacgcaattccgttatgctattccctatcgtatttaatgtgttatatttactatgttttagcgacaaccatcatacatacttcatctatgttgtataaaacatcttttccatgtgattgtgttcgttttgaaagttgtactaagagggctgtgtgcccattacccatcccttcctcacgcgcaggtgttccaactatctgggacccggcagggtgggtgtgtggctactagtgggagatgtttggctgaggtcaggccgtggcagggacggaagctttgagtgggttgtgcttgcagggagaccggcagccaggtcaggggtatgcccttgagacctgcccggccctccccgacccaggcctcgcccgccacccagcggtctcctggggagggggcgcagcaatctggaaagtgtctggtgtgcccctcctcgggaaacagtgaaagaggaaccaacaccagcgcggagctggcgctggcgctggcggggaagagggagatgcagggatgccagcgcgtttgtgagggctctgcagggcttgtgtgcctgggtgctcgtgaacggatgctcgtgaacggatgctgggtgcttgtgtgtgctacgcacttgtgaacgagtgtgtgtgtgagtggcgtgtgcacctctatatccaaatttagctgggggagggggagagagtgaagaccaaggtgtgggcaaaggagataattgcctccctgcgatgggagattacccaccctccttccccagaggtgatttataagttaaagataaagaataatcaagtcctggcaaggaaggacacggtgctgtggacctgattagaatctcgggatggaccagcagcggggagggcgggggaggggaaggggaggggaaggggaggggaagagggacgggaagccctgcgccccccccccgccccgcccaaccagttccaggctccctggggctcagcgtgtccgagcttggcccaaattacagccgatcggtccaccattatttctcccaacttttaatttctgcttccaaaggatcattgcccgcggtaattgcaaaggaccgccctgcgcgcggaacaggctccggggaccgcagtgtaaggcggcgcgcacacccgccggtgcacgcgcccgcgcgctcacaccctgaagaagcgcgcgcgctcgcactgccggcgcccaccagggtccacctgcgtctggctgctcgctgcgtgggcaggaggaggagggagaggagcgacccgccggggagctaccacgatcccgcactgggactccggacgccaggcccaatcctcgagggccgagggccgcgggccgcagggccctgtcctgcaaacaccgccgctgtagataaaacgtacccaaataaagactcaggccgcggggcttcctatttatccagatgtggagaagggggcatttgtcgtattctttttggaagaaacacacattttctccttacttgaagaggatcgattttatgtaggccgcctgctgtgtgtgcaatttgtcggtcccccttttgggggcgggttaagagggacaagcagtctctcattttcgattccacttctaatgagcggcccggggaccgccggctccgcccctctgctggcactccaggtcgggccctaatttgacgacgtgaaaggggcgggcgtgggaagggatggaggggcacagaccctgagcaagagagaccctggagacgcaaaaggagaccccaagcgggagcaagtgagagaggcgggaccgagacccacggaacgcgcgggagggggctgcagacccggcggagactcggagaatcgcgcgtgagaacctctccccccatccccaccccaactgccgaggggcgccagagctgtgggagccttcggagctcagggccctgcgcgctgggggcccggaggagaggccaggccagtcaccgcatcctgcgccgggatgtcccagccaactcttggcgctcgctggcccgcgcgccctggggtcgctgcggccccgccgcagattgatcccgcaattatttaaaagccggggctgtctcctcccccttagccaaggaggctggcgcgccgtttccggacgagcggaggcgcagcgacgcgccgctgaccaccacagccgcctgcggaccgctctgggctccgggctgcgtgtctgttcgtcccgcccggcggctccgtccgtgtgtcctgtgcggggtccgcctcctcggcccgggaccgttcgcagccaattaggcgcgcgcgctaacgagggcggcggagccccgcggccgcctgcgggcgcacgtgtgttcggcgtgtgggcgcgtgggcgcctccggcggggctgccataaatgcgcggaccgggcgccgcgggcgcctaaggcggcggcgaaggcagcaccggccaagcgcagggcgcggcgccgagagcgagcggagcagcggccgcctgcgcggggtcgtgtggaggtgcgggggccgccagggatgggagcccagtaggtggcggggcccgagccgccggagcggcgggaggccggcatgagcgctagcggccccgaggctcctggggacattcccgcgctaccgccgcctccccagccaggttcggggcccgcaccgcccgctcccgccgccgctgcccaggaagctatggacgggcgcgccgagctgccggcctttccccgcgctggagccccgccgctcgcggccagcgacactgtgcctgcggcgcccgagggggctggagcggcccggcccgcagcgcccctgcgccccacctccttctcggtactggacatcctggaccccaacaagttcaacagcaggaggcgtcgttgcgtgctgctgggcccagtggctcccgctgcgtgcgccccttgcgcttcggccccatgcgcccctgcacccgccgcctcgggacgcccgccacgcgcggaggagctggagcgccgcgccctcgccggcgccgggggagtcggagccgccggagctgagccgccgagtgagtaggatgcgcccggcaccaggtcgcagggacgggggaccggaggcttctatgcgtttacgaccctgggacaccgtggcgagttgggtctggcccacgccgaggctccgtggaaagttcgctgagcgccgggtccgcaccagcccacccgtggcctccatccgacccggactgcagggcctgcatcgccaggctcctctggagaagcttagccgggtcaggcccaggggctgcagggctgtgaggccggtccagggttcgcctccgcgcgtctccggcctagaagggtagttggtgcaggaggcgaggggccgctcgcggtggctggcccgtgacctccccgcccccactgtgcccgcccgaggcctccctccctccctcctttctccctctctctctttttttttggacaaatcagactaattgtgacaaaacgctggttatcgctggaaaaacaattaaatcccttcgattacagttaaggggaaatgtgcttagcggcggaaagcggccggtaatggggcggcccgcgcccccggtcgggtcgatatcccattacggcgcgtgcatatctctttcaagcaccttgcaaactccccccgaacatctaaattatagggtcaaaattcggataattactcgattaaaacctattacacttattaggggccgctattgatcgcgaattgcattcgacctgctcctgattgctttttgtgtcccctcctcctccctccaactttccggccctactgaagcacagggaggacagcgcggggcggaccgggctgggccgcggccaccggctctgcgcctggaggctgcgggccgcacctgcgcggggcctcttccgggtcggccgagatggagggcgaacaaatccggctgagtggaggggaccctacggactctgcggcggctgcgctcagagcctccacctgcggggagtctatggggcggaaggccgggatcccgctgaagccgggaaggcgcgcgcatactacagccggggagtcatctgggccaggcacccggggcagggctgggacggcgctcgcggtcgtctaggggcggatcctactcctgctgagggtcgcggggacccagaccacctgctggcctccatgccaggtggccagtcgccctggaggaatctgacaggagcgattggtcatttcttaccggagatttcggttccgacctggccggagagcaggccgcggctgccgctgggcagctctgtggagggcacctgccccgactcctcctgacttaggcccccagtgccggggcataggcgaccctgcctgggagagatgctttcttccaaatttcagagactcaaaatgcagtcacttcagggcctctgcttggaggatgcggagctgctgttcagactcggccgaccgggcctttaggccgcggggagccactccgacctgcacgcccgcccagccctcctggtgaggccgagcagggccctacccgcggcaactctgcgcaggggccgaggcgcgggcaggggagccggggctgaccctcgcgaccgaagctcagggcctggaggcttgaggtagggtcggggccagggcataggatttgggtcccccaagccccattctctcggccggctccggccccaccgccgggcctgttattaaatgcccggcaattggctggccctgcgccggcggtaattggggggtaatatacgccgaaataaattaaatggccttaactagtttgtacactacacaaaatgagtttaattaagtgtgtatcagccctgcggatcgatcagagcttccactccgctgtgatctccgcagccgggcccggcccgcagcacccgtccagccccgctcaggcaaaaacgcccggcgccgcgagaacgggcagcgcggggcgagggggaccctctccatagaaagaaacaagcaagggaccgagaggagacgcgggcggcgggagggaaggacaggccgaggggagggaaacgacagagcgaaggagagagagaggaggcgggtgacgaagcaggaggcgctgagcagggggcgggagggaaccctgggggaggaagcggggcgcgcggagggcgggatctgggaaccggccggtcctaactgccctgccctgtccttgtccctccctcccgcagacgccggcgaccccttcaaggcaggggaggccgagaccaacgacaccaacggctacagcagcggcggcggcggccacagcccgagcgcggacagcggggacgaggttcccgacgacgaggacgacgacgaggacgaagcgcccgagacggaggcggcgcgaggcgcggaggaggcgcggggaggcggcggcggcctcggggcccgcgggtcgggttgccagggcgcggctgagaccgacgcgtcccccggagccaccgtcgacgaggcagcggcccccggaccccgcgagaactcgcccgttgcccagggcccaccgggaggtgcagcggcgccggggggcgcggggaccaccccgcagggcacggcgacggcggcgaagcccaagcggaagcgcacggggtccgactccaagtccgggaagccgcggcgagcgcgcaccgccttcacctacgagcagctcgtggcgctggagaacaagttcaaggccacgcgctacctgtcggtgtgcgagcgcctcaacctggcgctgtcgctcagcctcaccgagacgcaggtgaagatctggttccagaaccgccgaaccaagtggaagaagcagaacccgggcgccgacaccagcgctccgaccggcggcgggggcggaccgggaccgggggccgggcctggcacggggctgcccggcggcctcagcccgctcagcccctcgccgcccatgggcgccccgctcggcatgcacggcccggccgggtacccggcgcacggccccggaggcctggtgtgcgccgcgcagctgccgttcctgtcgagcccggcggtgctctcgcccttcgtgctgggctcgcagacctacggggcgcccgccttctacgcgccgcacctctgagccccgtgccgctccgcctccgaggccgctgccgggcccggctgttttctacgattccagtcgttgactctgtacataaccggttgctacggaagagttttaaatttataaatgaccatgcgcgtcggggccgggcaggtggctcccgcgggctcggcccgctgcgaagcactttgcggaacttcactcaatcaataaaccgcaggcggccgcgcccccttctcccaggctcccgcgccgccctccgcccggccgggcccacccgtgaatcctctccggtggggaccaaggctcctcgggtgttggccttgagcgggactccagcccgttttcccaccggcctggcgtcccccggccggaattcaggccactctgctggtgcctgcccgtcgtcgtgggcgcaggggctgagcgcaggggctcacccgccaagctggctgaggctcaggtcccagcgtcacctccctgaccgccccctccccgtcccaaaaaagatcacaggtctcggaaagtgaccagctgcttcctcttcgcctctcctcttagccatggagctggtggggccgcggcattttccgcctcctgctccccacccctgcagcctgctccggtcccactcccctgccccacagccccgcaagggagcaccgggctaggcgtgtcagcttgggggcggtgtcagcttcggctactgcctggaggaagaagcgcctgcctgttttccccggggagaaggggctgcctgggctcagctacctgcccaagagcgtcccagcccattcagacccaggcccccaaaagtcgggtcgagtgctccctcttggagctgccacctctcctccaacgacgctggaaacagtccctacatcccctcacaggctctcggagtcctgtgggtttggtgttcgcctgagctgggctcagggaggtgagaggcagaggcgcttgggctgggcggcagtgtggatgcaaaagctggtgcttggaagcggggaagggcgacgggaggagggttcagagccccagaaagggtggggaggcggcatgagacactggccacagccgggcaggtgctgcccctcttctgccactggcgggagtcctgagtcctgcctcacagagggagcttggctctgcctggcggctgtgccctgactgggcccagtctcctggtgtcctgtccattttccgtgagtgggctgagctcatctctgggctgtcaacctgacagcaaccactgggggagggctgggggcaggtggcggcagtgaatacccagtgccacaggcaagatgggagacactgctgcccactggcctggccagcacctgtggacagagagcaggcccagagcagcaccggccatcgggcaggttgagggggcggggcgcaggggcggctgggatcaccctgtgtgtttgctgcccccacaccccaagccccaggcaggccttggttcctccagccaagggccggcctgactcccagttgctctctgctccctttctgggtccgcataggtgaagtcacctgcctgcagcaagcgtcctgaggctcaccagcccgggggcagggcagcctctccctcctccctcctccctcctccctcctccctccgctggcagagctcagacttgctcggtgggggtccatgagggaaggcgagccacctgcaggtggccaagtgtagccagagggtcgactcacagctgaagccagtgcagcgggcttggcctaagctgggcactggggctgctgagtgggaaagcagctgctgcgtggccaggaggaacccaggtctgtgctgcaagggccttcccctttgctgagcttgctggaacattccaggagctcacagccccacagggagatgatagaaggtggctgcgacggtgcaggccagattcagacccactcagagccgggccagaccgctgtgtgcagccctcccatggagcttagatcctggctccccgtccctccagcttccacatgcccccaccctggcctcctctgtctcctcccctcaagcctcagccagaaacacagagccacaccggagggccctctagcagtgcccctgggctgcaggatgggatggagggtatgggccgggggctgtgaggtctgggaagcaatacccccctccttgcaccaccccttctctaagctccctggacagcccacagcctcatgctgcaggcatggggggcatttcctccagtctctgattcacagtcactctgcagcggaccctcgtcggagtcggactgcgtgcaaagccacctgagcctcccactgtgctttgctgagagtcacatgtgggaaactgaggcccggcagtcatggggcttgctgggggcagtgccagctctggtgccagtgggtgccctgctccctcctgaaggagcctccactttttagcccacactcctactgggtccccaggcccccagtctccctgggagagttggggagccattcccttgcctgcctcccagggagccctcgggctgccccaccacccccatttccaccaggtttgggcctcccctgcccgccatttccagtctaggctgcctgtgcctgactcagtttcagctccctgggcctgtgctgcaggcagaggtgagcgggagccttgttaggggtctcaacgtgtcccccactttggtgtcagggccgtgagggaaggtctcaacctcaggatctgagggcactcttccatggaccctcctcctagggctcaggacccatcttgccctggaccacacctgccccaagttcccagaaccacacacatgtcgttgctgcaaagccaggcacagaaggggcctcttgtcaagcgagcatgagcccagacaagggcggcgtttgctctcacgccctgggctcccgtctcctgcccagggaccgctgagcccagacggtaaggggcctcccattgaggccacctccccacccccaccagccgatggtgctgtggtccattgctgtgtgggtcaggcctgggtttggctgcgtgtaagagaaaacctgaatgaagagagttgaaaggagactgtttactttctctttgtgagggaattctggccgtggcagccctgggcagttttgagaccttcactgagtccttggagacccagctcctggctctctgtgctgcatcaggcctggacgggcctcagcctcacagaacccaatggaaactgaggctccagccaccacatcctcacccccggctgcaaacaggaagaaagggagtggccagcaacacccctctcttttaaggagctttcccggaagcccacacaacacagctgcttatgtcccattggccagagctgggtcacgggccctctcttagccaagggaggctgggaagtgcagtctccccccaggcatctcactcctagtgaaaggtgaggatggattggggccaccggctgcccctgccgagcccccctgtgctccccacccatgcccacctgcccacacccccagccccaggcagctgggcttctgaagaaagtcgccctcagcccgcctggtcttttccgaagcccagggcctcgaccttggtgggcacccagctcctacaccctaaggtgtctggtgtaccctctccataaccccaccctgggttggccacaggtccaagcccatcttccagccataggatcttgtcacctgcctgcgtcctgccaggggagacagcccctgctgggcttcagatgcgtccagtgtctccaggcttaggcataaccctccctggaccccgtgtggcctccagctctccccctttcctcccagcctccagccaaagtatcgaaagagccgtctacacctgctgtctacacttcctcctcacttctccacccctaaggaggctctcgccgtcactcgggcccttggctgccccatcctggcgtggcctcctcagccccctgctcacttggtggctggttcctcctgctccgagtcctgggcagcctgggcttggggaaccctcatcctccacttgccttcctgccatggcaatggtgtgcctggccctgtccttgccccgcgatgtcctggcaatggtgtgcttctccctgtccttgccccgcagtgtcctggtaatggtgtgcctcgccctgtccttgccctgcgatgtccccccgggggccaggggccctccaggcaccacaaagccagaccaagctcctcccagaccaagccagccccacagcctcagagcaaaccccaggcaccaagcaccctccccacatccaacccaatctacccatatccagatgcccgaaaacaccctgcatccaacctgatctacccacatccagatgcccgaaaacacccggcatccaacctgatctactcacatccagatgcccgaaaacaccctgcatccaacctgatctacccacatccagatgcccgaaaacacccggcatccaacctgatctacccacatccagatgcccgaaaacacccggcatccaacctgatctacccacatccagatgcccgaaaacacccggcatccaacctgatctactcacatccagatgcccgaaaacaccctgcatccaacctgatctacccacatccagatgcccgaaaacacccggcatccaacctgatctactcacatccagatgcccgaaaacaccctgcatccaacctgatctacccacatccagatgcccgaaaacacccggcatccaacctgatctacccacatccagatgcccgaaaacacccggcatccaacctgatctacccacatccagatgcccgaaaacacccggcatccaacctgatctacccacatccagatgcccgaaaacaccctgcatccaacctgatctacccacatccagatgcccgaaaacaccctgcatccaacctgatctacccacatccagatgcccgaaaacaccctgtatccaacctgatctactcacatccagatgcccgaaaacacccggcatccaacctgatctacccacatccagatgcccgaaaacaccctgcatccaacctgatctacccacatccagatgcccgaaaacacccggcatccaacctgatctacccacatccagatgcccgaaaacaccctgcatccaacctgatctacccacatccagatgcccgaaaacaccctgcatccaacctgatctacccacatccagatgcccgaaaacacccggcatccaacctgatctacccacatccagatgcccgaaaacacccggcatccaacctgatctacccacatccagatgcccgaaagcacccggcatccaacctgatctacccacatccagatgcccgaaaacacccggcatccaacctgatctacccacatccagatgcccgaaaacacccggcatccaacctgatctacccacatccagatgcccgaaaacaccctgcatccaacctgatctacccacatccagatgcccgaaaacaccctgcatccaacatcccccctggccaccgccacgttgtccccccatccctaggacctccctgcggtgctcaggcctccccacacccagtgtcctgcttgtgatggccacctgggtcccccatctcttccctggtgactgagtgccccctcactcctggtctcttctggccagctgtgcacagcgtcagctgggaggcggggaggtgaggctggacgaatagctcagcagcagagggaggtgagcacagggaggaaggggcaggggtgggcagccaggcagggctgcaggactgcacctgagggaggaggcagggctgagccccatccctgccctcacgaccactgtggcccccacctgcctctggcactgttgcccctcccccatcccaaggtcaccttttcatccacagaggcccgtggggtgggggcatctcatccactggggggggaccttgctcatgggtgacaacctggggatgtccatgtcccacctcctcccgactgccctgcagtgtggtcccctttggagacagctctgctggtgcagacgccttctctctttccctgggaggctgtggctgagcgctcatctcagatgagctgagctccacatgtcctgcctctggggcagcaggcagctgggtgctgcctgtgactaggttctggccaagtcgacatgaacagtgtgtgtgggggtaggggactgagaaggctccttatgggaatcagctcagaggggaggcaaagccctcctccttccttctgcagacctggtgatggctggagccccagcagccaccttgggccatgaggtagccttggggttggaggccacgtgctctgatactgagccaggaactggaaggtgcttgggggcgcctacccttggaccctgattcaataggacacaaacccttctgtctacgagccactcttatttggagtttctggttctgtgttgctaaccctgacaccactttccatggtggcccatccaccccagcgctggggctgtgacccatgcccaccagtcctcccttggtcactcctcgcactcaccccctgctgctctccgggccaacctcagccagtctgccctggatgagggcagcagcatgctggaccagctggcacgggccccctcccactgtgagccgtaccagtagcccctacgtgcatctgcaggccagacccccccaggctcctccccccgccatctgcctggctcatggggacccaaaccactttgtggcaagtgtgggtggaaggggtttgctgaggggatctgaggagggcccacccccagcacccccttagtgccaagtcactgacagtgccctgagcccgcagtgctggagctgctcccaggtgcacaggccccacccctcaccccaccagccagaagggccggcccagcacccccaccccacagggcccctcacttggtcacaaacactgagtcacaggtttgtcttttaatcagcatccatgaaaattcattaaaaggtctgatacgaaaagcaccaggctaacaaatcagtcctgaattcaattttacaaaaacaccctctcctcttccatctcatccctaatattcctacgttatctcgaggtgcagagaaatataaaagggatccttgacataaaaacgtattaaaaagccaccatgagtagaatgtacggtaacagcgtctgactcggccggcaggacaggaaacctacagaaccaccatgccagggctttgtgtgagcaggcgggcactccacatgggcacgtgtgtgagcaggcaggcactccacatgggcacgtgtgagcaggcgggcactccacatgggcacgtgtgtgagcaggcgggcactccacatgggcacgtgtgagcaggcgggcactccacatgggcacgtgtgagcaggcgggcactccacatcggcatgtgtgtgagcaggcgggcactccacaatggcacgtgtgagcaggcgggcactccatatgggcacgtgagcaggcgggcactccacgtcggcacgtgtgagcaggcgggcactccacatgggcacgtgtgagcaggcgggcactccacatcggcacatgtgagcaggcgggcaccccacatcggcacgtgagcaggcgggcactccacatgggcacgtgtgagcaggcgggcactccacatcggcacgtgagcaggcgggcactccacatgggcacgtgtgagcaggcgggcactccgcatcggcacgtgtgagcaggcgggcactccgcatcggcacgtgtgagcaggcgaacactccacatgggcacgtgtgagcaggcaggcactccacatcggcacgtgtgagcaggcgagcactccacatgggcacgtgtgatcaggcgggcactccacgttggcacgtgtgagcaggc

SEQ ID NO: 4 - DNA, 3168 bp, Mus musculus
atggaccagcgcggatcattgcccgcggtaattgcaaaggaccgtccttgtgcagagcagtacctggtcgcgcctagtaacgggcgccattaacgtttcctggcaggaacacgtctcgtcgctctcgggaccgtagtgggaggaggaatgcatacccgctggagcacacgtcgtaggcgccgagagccctggcatcaccctcctccttacgtatgggcgacctcgtgtgcagcatccgcgacacactcacactctgggcacgccagttccatcgccgggtcccacctgggtccttcagagtgtgtgagtgtgagacccgtgcggtcaaggtagcggcccagggtggacccaggaagtctcgcgggtcagctcattggcctgagcgagaggaggaggaaaactaggaagcgaagcatcggccgcccagtcgagtaaccggactcgctctcctcctccttttgatccttcgcttcgtagccgtacctgaccctcatcctccaaagccgggctcgaccttccgaggtcgtccgccgagactgtatggactgggagtaggaggtttcggcccgagctggaaggctccagcaggcggctctgacacctgtaaatctccagactggacaagacgagctagaacgccgagccctctccgccgccacaggacatttagaggtctgacctgttctgctcgatcttgcggctcgggagaggcggcggtgtggagtcgcagcggccgcgggagctgagcctacaaggccctgcttagaggttcacaatacgcctcagcgtcgccggcgccctcgactcggatgttccgggacgaatctccaagtgttatgcctttcccagcctccaaactcaaccagcccacgggaagctggagcccgcttaagacagagtgaaagggtcggaggtttgagttggtcgggtgcccttcgacctcgggcgaattctgtctcacgctggggagctggttgccctaaaggccaaggccaggactgcggatggggcagcaaagcagcgacccctcgaccaacgggatttccggttccggtcctgacgcctaccccgtcgtttcgtgctcgacacggagtgggtccttccgaaggaaggcagagaggaaatgcaagcaatcaaggacgagctgtgcctcacccaggaaggcttccttccgtctctcctttacgttcgttagttcctggaactgtggcaaggaggaagaaaaagaaaatctttggtgctggggactcttacagggcgccttgacaccgttcctccttctttttcttttagaaaccacgacccctgagaatgtcccgcgacgaggccgaggccaacggctacagcagcggcagcggtcgcagcccgaccgcggacagcctgctccggctccggttgccgatgtcgtcgccgtcgccagcgtcgggctggcgcctgtcggaggatgaagcgccggaggacgaggacgaggaagaagcgcccgaggtgcaggatgctcagctcctacttcgcggcctcctgctcctgctccttcttcgcgggctccacgtcctacgagtcggcacggaggagcctcggggaggcagcggcggtctcggggcccgcgggtcgggctgccccccgtgcctcctcggagcccctccgtcgccgccagagccccgggcgcccagcccgacggggggagcggccgaggtcgaggcttcccctgtggacgatactgcggccccagggccccgtgggcctcgccggctccagctccgaaggggacacctgctatgacgccggggtcccggggcacccaactcgcccggagccccgggcccgcctgcaactgctacgggagcggggagcgcggggagtttgagcgggcctcggggcccgggcggacgttgacgatgccctcgcccctcgcgcccctcaaccccgcagggcgccgccgtgaccaccaagcccaagcggaagcgcacgggctcggactcgtggggcgtcccgcggcggcactggtggttcgggttcgccttcgcgtgcccgagcctgagcaagtccgggaagccgaggcgtgcgcgcaccgccttcacctacgagcagctcgtggcgctgttcaggcccttcggctccgcacgcgcgtggcggaagtggatgctcgtcgagcaccgcgacgagaacaagtttaaggccactcgctacctgtcggtgtgcgagcgcctcaacctggcgctgctcttgttcaaattccggtgagcgatggacagccacacgctcgcggagttggaccgcgactcgctgagcctcaccgagacgcagcccggccgtgctgtcgcccttcgtgctgggctcgcaagcgactcggagtggctctgcgtcgggccggcacgacagcgggaagcacgacccgagcgtgacgtacggcgcgcccgccttctatgcgccccacctctgaatctcggtgccaggaccccgctgcatgccgcgcgggcggaagatacgcggggtggagacttagagccacggtcctggggcgtggggaccgcagcacgcggcctcaaggccagaggacccaaggcgagcatgggcgtctctcacccctggcgtcgtgcgccggagttccggtctcctgggttccgctcgtacccgcagagatttctaaggtttcagtcgttggtcctgtatatagatagccgttcctacagaagagaaattaaagattccaaagtcagcaaccaggacatatatctatcggcaaggatgtcttctctttaatataaacgagtgtgcgcaggggccaaacgggaggctcccgtggcctgctccaagcactttatatttgctcacacgcgtccccggtttgccctccgagggcaccggacgaggttcgtgaaagtagaactcttaataaagcacaggaagccatgcgggcgttttcccgaaggctctggcgcccatcttgagaattatttcgtgtccttcggtacgcccgcaaaagggcttccgagaccgcgggtaggatgccgatcccgccttcctgtccgctccggttcttatccatctgtgtgtcgccctcatcctacggctagggcggaaggacaggcgaggccaagaataggtagacacacagcgggagggcagggacttcagagcggttagcccgtccctgaagtctcgccaatc

SEQ ID NO: 5 - DNA, 25 bp, Artificial sequence
  Synthetic primer
ccgggtacgtaggaattccatatgc

SEQ ID NO: 6 - DNA, 22 bp, Artificial sequence
  Synthetic primer
aggcgctgacaccagcgcgccg

SEQ ID NO: 7 - DNA, 25 bp, Artificial sequence
  Synthetic primer
tcctggggcggagcgggcagggcgg

SEQ ID NO: 8 - DNA, 1488 bp, Mus musculus
cctgtggtgcccgctacgtgtgccccatgcgccccggccgcgtgcgtcgcggtccccgctggacaccacgggcgatgcacacggggtacgcggggccggcgcacgcagcgccaggggcgagcctccggacgttcgccgcgcgcagagctagaacgccgagccctctccgccgccacaggacggaggcctgcaagcggcgcgcgtctcgatcttgcggctcgggagaggcggcggtgtcctgtcgcagcggccgcgggagctgaggtgctggggactcttacagggcggacgaggccgaggcagcgtcgccggcgccctcgactccacgacccctgagaatgtcccgcctgctccggctccccaacggctacagcagcggcagcggtcgcagcccgaccgcggacagcgaggatgaagcgcggttgccgatgtcgtcgccgtcgccagcgtcgggctggcgcctgtcgctcctacttcgcgcggaggacgaggacgaggaagaagcgcccgaggtgcaggatgctcagggcacggaggagcgcctcctgctcctgctccttcttcgcgggctccacgtcctacgagtcccgtgcctcctcgctcggggaggcagcggcggtctcggggcccgcgggtcgggctgccccggagcggccgagggagcccctccgtcgccgccagagccccgggcgcccagcccgacggggcctcgccggctcctcgaggcttcccctgtggacgatactgcggccccagggccccgtgggaactcgcccggagagctccgaaggggacacctgctatgacgccggggtcccggggcacccttgagcgggcctcccccgggcccgcctgcaactgctacgggagcggggagcgcggggagtaccccgcagggcgggggcccgggcggacgttgacgatgccctcgcccctcgcgcccctcatggggcgtcccgcccgcccgtgaccaccaagcccaagcggaagcgcacgggctcggactcgaagtccgggaagggcgggcactggtggttcgggttcgccttcgcgtgcccgagcctgagcttcaggcccttcccgaggcgtgcgcgcaccgccttcacctacgagcagctcgtggcgctggagaacaagtttggctccgcacgcgcgtggcggaagtggatgctcgtcgagcaccgcgacctcttgttcaaaaaggccactcgctacctgtcggtgtgcgagcgcctcaacctggcgctgtcgctgagcctcttccggtgagcgatggacagccacacgctcgcggagttggaccgcgacagcgactcggagaccgagacgcaggtgaagatctggttccagaaccgccgaaccaagtggaagaagcaaaactggctctgcgtccacttctagaccaaggtcttggcggcttggttcaccttcttcgttttgccaggcgctgacaccagcgcgccgggtccgcgactgtggtcgcgcggc

SEQ ID NO: 9 - protein, 248 aa, Mus musculus
PVVPATCAPCAPAACVAVPAASGRSPRAELERRALSAATGVAAAAGAEVLGTLTGRTRPRPTATAAAAVAARPRTARMKRRRTRTRKKRPRCRMLRARRSLGEAAAVSGPAGRAAPERPRSRLPLWTILRPQGPVGTRPEPRARLQLLRERGARGVPRRAPPVTTKPKRKRTGSDSKSGKPRRARTAFTYEQLVALENKFKATRYLSVCERLNLALSLSLTETQVKIWFQNRRTKWKKQNPGADTSAP

SEQ ID NO: 10 - protein, 198 aa, Homo sapiens
MRSCCSDSADRAFRPRGATPTCTPAQPSCPILSAGSGPTAGPVIKCPAIGWPCAGGNWGCTATAAKPKRKRTCSDSKSGKPRRARTAFTYEQLVALENKFKATRYLSVCERLNLALSLSLTETQVKIWFQNRRTNPLSPSPPMGAPLGMHGPAGYPAHCPGGLVCAAQLPFLSSPAVLSPFVLGSQTYGAPAFYAPHL

SEQ ID NO: 11 - DNA, 1584 bp, Mus musculus
atggaccagcgcggatcattgcccgcggtaattgcaaaggaccgtccttgtgcagagcaggctctcgggaccgtagtgggaggaggaatgcatacccgctggagcacacgtcgtaggcgcacacactcacactctgggcacgccagttccatcgccgggtcccacctgggtccttcagaggcgggtcagctcattggcctgagcgagaggaggaggaaaactaggaagcgaagcatcggctacctgaccctcatcctccaaagccgggctcgaccttccgaggtcgtccgccgagactgtcctgtaaatctccagactggacaagacgagctagaacgccgagccctctccgccgccacaggagtcgcagcggccgcgggagctgagcctacaaggccctgcttagaggttcacaatacgctttcccagcctccaaactcaaccagcccacgggaagctggagcccgcttaagacagagtcgctggggagctggttgccctaaaggccaaggccaggactgcggatggggcagcaaagcagctcgacacggagtgggtccttccgaaggaaggcagagaggaaatgcaagcaatcaaggaggaactgtggcaaggaggaagaaaaagaaaatctttggtgctggggactcttacagggcggacgaggccgaggccaacggctacagcagcggcagcggtcgcagcccgaccgcggacagcgaggatgaagcgccggaggacgaggacgaggaagaagcgcccgaggtgcaggatgctcagggcacggaggagcctcggggaggcagcggcggtctcggggcccgcgggtcgggctgccccggagcggccgaggtcgaggcttcccctgtggacgatactgcggccccagggccccgtgggaactcgcccggagccccgggcccgcctgcaactgctacgggagcggggagcgcggggagtaccccgcagggcgccgccgtgaccaccaagcccaagcggaagcgcacgggctcggactcgaagtccgggaagccgaggcgtgcgcgcaccgccttcacctacgagcagctcgtggcgctggagaacaagtttaaggccactcgctacctgtcggtgtgcgagcgcctcaacctggcgctgtcgctgagcctcaccgagacgcagcccggccgtgctgtcgcccttcgtgctgggctcgcagacgtacggcgcgcccgccttctatgcgccccacctctgaatctcggtgccaggaccccggtggggaccgcagcacgcggcctcaaggccagaggacccaaggcgagcatgggcgtctcttttctaaggtttcagtcgttggtcctgtatatagatagccgttcctacagaagagaaatttataaacgagtgtgcgcaggggccaaacgggaggctcccgtggcctgctccaagcactttgtagaactcttaataaagcacaggaagccatgcgggcgttttcccgaaggctctggcgccgtaggatgccgatcccgccttcctgtccgctccggttcttatccatctgtgtgtcgccctgggcagggacttcagagcggttag

SEQ ID NO: 12 - protein, 527 aa, Mus musculus; identical to SEQ ID NO: 2